(19) United States
(12) Patent Application Publication
(10) Pub. No.: US 2020/0246386 A1
(43) Pub. Date: Aug. 6, 2020

(54) METHODS AND COMPOSITIONS FOR TREATING NEUROLOGICAL DISORDERS

(71) Applicant: PLURISTEM LTD., Haifa (IL)

(72) Inventors: Rachel OFIR, Adi (IL); Niva SHRAGA HELED, Nahariya (IL)

(73) Assignee: PLURISTEM LTD., Haifa (IL)

(21) Appl. No.: 16/607,112

(22) PCT Filed: Apr. 23, 2018
(86) PCT No.: PCT/IB2018/052806

§ 371 Date: Oct. 22, 2019

Related U.S. Application Data
(60) Provisional application No. 62/488,883, filed on Apr. 24, 2017.
(60) Provisional application No. 62/581,718, filed on Nov. 5, 2017.

(51) Int. Cl. A61K 35/28; C12N 5/073; A61P 25/28; A61P 25/16

(52) U.S. Cl. CPC A61K 35/28; C12N 5/0605; A61K 9/0019; A61P 25/16; A61P 25/28

(57) ABSTRACT

Disclosed herein are methods and compositions for cell induction and treating neurological disorders, utilizing adherent stromal cells, which may, for example, be derived from placental tissue, bone marrow, or adipose tissue. Also provided are pharmaceutical compositions comprising the described cells, optionally in combination with pharmaceutically acceptable excipients.

### FIELD

Disclosed herein are methods and compositions for cell induction and treating neurological disorders.

### BACKGROUND

Neurodegenerative diseases are debilitating conditions, often incurable, that result in progressive degeneration and/or death of neurons, resulting in motor problems (ataxias), and/or deficiencies in mental functioning (dementias). Examples of neurodegenerative diseases are Alzheimer's disease; Parkinson's disease; Amyotrophic Lateral Sclerosis (ALS); Huntington's disease; spinal muscular atrophy (SMA); multiple sclerosis (MS); and ataxia-telangiectasia.

### SUMMARY

Disclosed herein are methods of treating a neurological disorder, in a subject in need thereof, comprising administering to the subject a pharmaceutical composition comprising adherent stromal cells (ASC), thereby treating a neurological disorder. In certain embodiments, the neurological disorder is a neurodegenerative disorder. In certain embodiments, the ASC are placenta derived, while in other embodiments, they are adipose derived. Alternatively or in addition, the ASC have been induced.

In certain embodiments, the ASC described herein have been cultured on a 2-dimensional (2D) substrate, a 3-dimensional (3D) substrate, or a combination thereof. Non-limiting examples of 2D and 3D culture conditions are provided in the Detailed Description and in the Examples.

Unless otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

All colors mentioned in the figure legends refer to original color images.

FIG. 1 is a diagram of a bioreactor that can be used to prepare the cells.

FIG. 2 is a table showing various combinations and concentrations of factors tested for their effects on BDNF secretion, and the test results. The final process used in many subsequent experiments was similar to the conditions depicted in the bottom row, except that a 1-day incubation and the optional addition of serum was subsequently used.

FIGS. 3A-C are plots of the concentrations of various factors in the conditioned medium (CM) of ASC induced as described herein. Concentrations are shown on the vertical axis and are expressed in pg/ml (picograms per milliliter). Black and gray bars in each series depict non-induced ASC and induced ASC, respectively. Factors are grouped by expression level, as follows: A. Medium, from left: LIF, BDNF, GDNF. B. High, from left: VEGF, G-CSF. C. Very high: IL-6, HGF, IL-8.

FIG. 4 is a plot showing time course of BDNF secretion from induced ASC. Three different batches of ASC (horizontal axis) were induced, then CM was collected immediately (solid bars); or cells were incubated in growth medium supplemented with 1% HS (human serum) for 24, 48, or 72 hours (white, vertical-striped, and horizontal-striped bars, respectively). Vertical axis shows BDNF concentration in pg/ml.

FIG. 5 is a plot showing effect of serum on induction of ASC. ASC were incubated without induction agents (left set of bars) or with induction agents (right bars) in medium without serum or with 1% or 10% serum (solid, white, and striped bars, respectively). Vertical axis: BDNF concentration in pg/ml.

FIG. 6A-B are microscopy pictures showing that CM from induced ASC stimulates neuronal differentiation of SH-SY5Y neuronal precursor cells. A shows SH-SY5Y cells incubated with regular growth medium, and B shows SH-SY5Y cells incubated with CM from induced ASC. Upper panels show cells stained for human nestin (left; red), human βIII-tubulin (second from left; red), and human tyrosine hydroxylase (third from left; green), and human choline acetyl transferase (right; green), and lower panels show phase contrast images. Nuclei are stained with DAPI (blue). C-D are grayscale versions of the original color Figs. A-B.

FIG. 7 is a plot showing BDNF concentration in CM collected from cells following incubation under various conditions, after seeding at 0.4, 0.8, or 2.9×10\hat{ }6 cells and growth for 5 days (bars 1-3, 4-6, and 7-8 from left, respectively). For the left and middle sets, the left, middle and right bar within each set depicts incubation with no induction agents, or with induction agents for the last 24 hr. or the last 72 hr., respectively. For the right set of bars, the left bar depicts no induction agents, and right bar depicts a 72-hr incubation with induction agents. Cells seeded at 0.4 and 0.8×10\hat{ }6 cells/flask were grown for the whole period in DMEM+20% FBS, whereas cells seeded at 2.9×10\hat{ }6 cells/flask were grown in basal DMEM supplemented with glutamine and antibiotics for the last 72 hr. Vertical axis: BDNF concentration in pg per 10⁶ cells.

FIGS. 8A-D are plots showing BDNF concentration in CM collected from cells, after seeding at 0.4, 0.8, or 2.9×10\hat{ }6 cells (A, B, and C, respectively), grown with or without serum supplementation which were or were not induced for 24 or 72 hours (as described for FIG. 7), following which the cells were cryopreserved, thawed and seeded equally in 6-well plates for 72 hours (0.5*10⁶ cells/well), and medium was sampled after 24, 48, and 72 (left, middle, and right bar, respectively, in each series). Cells seeded originally at 0.4 and 0.8×10\hat{ }6 cells/flask were grown for the whole CM sampling period in DMEM+20% FBS, whereas cells seeded at 2.9×10\hat{ }6 cells/flask were grown in basal DMEM supplemented with glutamine and antibiotics for the last 72 hr. Different sets of bars depict different induction conditions (before cryopreservation). Namely, for A-B, the left, middle, and right sets of bars depict incubation with no induction agents, or with induction agents for the last 24 hr. or the last 72 hr., respectively. For C, the left and right sets of bars depict incubation with no induction agents or with induction agents for the last 72 hr., respectively. D depicts BDNF data from the 72-hr. timepoint of the experiment described above, but normalized to the number of cells that were harvested 72 hr after cell thawing, Vertical axis: BDNF concentration in pg/ml (A-C) or pg per 10⁶ cells (D).

FIGS. 9A-C are plots showing concentrations of high-, medium-, and low-expressed cytokines (A, B, and C, respectively) in CM collected from ASC after cryopreservation. Horizontal axis indicates the measured cytokines for each set of 3 bars. For each set of bars, the left, middle, and right bars indicate incubation with no induction agents (solid bars), or 24-hr (white bars) or 72-hr (striped bars) incubation with induction agents, respectively. The rightmost bar for osteopontin and the leftmost bar for GCP-2 in A are barely visible, reflecting miniscule values. The rightmost bar for IGFBP3 in A is invisible, reflecting a possible zero value. Vertical axis: cytokine concentration in pg/ml.

FIGS. 10A-C are plots showing concentrations of high-, medium-, and low-expressed cytokines (A, B, and C, respectively) in CM from induced and bioreactor-expanded ASC after cryopreservation. Vertical axis: cytokine concentration in pg/ml. Horizontal axis indicates the cytokine measured; the last set in A is osteopontin. Most sets of bars contain bioreactor-expanded ASC (solid bars), or ASC incubated in DMEM+20% FBS in the absence of induction agents (white bars) or with induction agents at regular (vertical stripes) or high (i.e. 5× concentration of N2 supplement and bFGF) (horizontal stripes) concentrations. Only some cytokines were measured for the high-concentration induction agent conditions, namely the leftmost 3, 1, and 5 cytokines depicted, respectively in panels A, B, and C.

FIGS. 11A-B are fluorescent microscopy pictures showing the effect of CM from bioreactor-expanded ASC on SH-SY5Y differentiation. The lower right panel depicts SH-SY5Y incubated in negative control medium (i.e. normal growth medium for SH-SY5Y cells), and other panels depicts SH-SY5Y incubated in CM from ASC batches 1-5. Upper panels from left to right are batches 1-3, and lower left and center panels are batches 4 and 5, respectively. In (A), Beta III tubulin (a mature neuron marker), ChAT (a cholinergic neuron marker), and DAPI (nuclei) are stained in red, green, and blue, respectively. In (B), Nestin (an immature neuron marker), TH (a marker of dopaminergic or noradrenergic neurons), and DAPI are stained in red, green, and blue, respectively. E-F are grayscale versions of the original color Figs. A-B.

FIGS. 11C-D are fluorescent microscopy pictures showing the effect of ASC CM on SH-SY5Y differentiation. The left 2 panels are batches 1 (top) and 2 (bottom) of ASC incubated in DMEM+20% FBS without induction agents, and the middle 2 panels are the same batches incubated with induction agents. The right panel depicts SH-SY5Y incubated in negative control media. Staining in (C) and (D) are same as FIG. 11A and FIG. 11B, respectively. G-H are grayscale versions of the original color Figs. C-D.

FIG. 12A contains microscopy images depicting staining of undifferentiated neurons (negative control; upper left panel), or neurons differentiated with 1 mM cAMP (upper middle panel), 10 mcM butyric acid (positive control; upper right panel), or CM from bioreactor-expanded ASC (lower panels). Cells are stained for human βIII-tubulin (red) and human tyrosine hydroxylase (green). Nuclei are stained with DAPI (blue). C is a grayscale version of the original color (A). B depicts the relative percentage of differentiated neurons (vertical axis) in SH-SY5Y cells untreated or exposed to butyric acid, cAMP, or ASC-derived CM from bioreactor-expanded ASC or ASC induced with induction agents, respectively (bars ordered left to right). The depicted percentages are the averages of 4 different batches.

FIG. 13. CT images of GNP-stained cells in murine brains. GNP stained cells are seen as green dots in original color images. Depicted are CT images including the brain area of intra-nasally injected mice (A) and IV-injected mice (B). Coronal (left) and sagittal (right) views are shown for (A); coronal view is shown for (B).

FIG. 14A is a plot of viability of differentiated SH-SY5Y cells. Cells were pretreated with either control medium (white circles), or CM from placental ASC subjected to either bioreactor expansion (black circles, solid line) or incubation with bFGF, N-2 supplement, heparin and cAMP (black circles, dotted line) Two hours later, H₂O₂ (200 μM) was added to each medium. Luminescence (vertical axis) was recorded every 15 minutes for 8 hours and is expressed as percent of control, which is the cells exposed to same medium without H₂O₂; horizontal axis shows time (hours) after the addition of H₂O₂. B. Column chart at the 6.5-hour timepoint for the experiment described for A. Black, white, and gray bars show control medium, bioreactor-expanded, and induction agent groups, respectively C. Plot showing level of reactive oxygen species (ROS) in differentiated SH-SY5Y cells, after treatment with H₂O₂ (200 μM) in either control medium (white circles), or CM from placental ASC subjected to either bioreactor expansion (black circles, solid line) or incubation with bFGF and cAMP (black circles, dotted line). Fluorescence (vertical axis) was recorded every 15 minutes for 6 hours and is expressed as percent of control, which is the cells exposed to same medium without H₂O₂; horizontal axis shows time (hours) after the addition of H₂O₂.

FIGS. 15A-B are plots showing the effect of intrathecal (IT) injection of bioreactor-expanded ASC on the mass (A) and disease score (B) (vertical axes) of transgenic familial ALS mice. Horizontal axis indicates number of days following IT injection.

FIG. 16A is a perspective view of a carrier (or “3D body”), according to an exemplary embodiment. B is a perspective view of a carrier, according to another exemplary embodiment. C is a cross-sectional view of a carrier, according to an exemplary embodiment.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Provided herein are methods and compositions that comprise adherent stromal cells (ASC) that have been treated to make them suitable for treating a neurological disease, for example a neurodegenerative disease. Such treatment is referred to herein as “induction”, and the cells so treated as “induced” cells. As described herein, induction can comprise, in various embodiments, incubation in medium comprising induction agents, expansion on a 3D substrate, or a combination thereof.

In certain embodiments, the induced ASC secrete neurotrophic and neuroprotective growth factors. In more specific embodiments, the neurodegenerative disease is Alzheimer's disease; is Parkinson's disease; is Amyotrophic lateral sclerosis (ALS); is Huntington's disease; or is SMA, each of which represents a separate embodiment. In still other embodiments, the neurological disease is multiple sclerosis (MS), or is ataxia-telangiectasia.

ASC and Sources Thereof

“ASC”, unless indicated otherwise, refers to adherent stromal cells before induction, as described herein. In some embodiments, the ASC may be human ASC, or in other embodiments animal ASC. In some embodiments, the ASC are allogeneic, while in others, they are autologous. In certain embodiments, the ASC are placenta-derived; while in other embodiments, they are adipose-derived; which in other embodiments, they are derived from another tissue.

In certain embodiments, the described ASC are mesenchymal stromal cells (MSC). These cells may, in some embodiments, be isolated from many adult tissues, such as placenta, bone marrow and adipose. In further embodiments, the cells are human MSC as defined by The Mesenchymal and Tissue Stem Cell Committee of the International Society for Cellular Therapy (Dominici et al, 2006¹), based on the following 3 criteria: 1. Plastic-adherence when maintained in standard culture conditions (a minimal essential medium plus 20% fetal bovine serum (FBS)). 2. Expression of the surface molecules CD105, CD73 and CD90, and lack of expression of CD45, CD34, CD14 or CD11b, CD79α or CD19 and HLA-DR. 3. Differentiation into osteoblasts, adipocytes and chondroblasts in vitro. In more specific embodiments, the ASC are placenta-derived, or, in other embodiments, are adipose-derived.

Alternatively or in addition, the referred-to ASC are mesenchymal-like ASC, which exhibit a marker pattern similar to mesenchymal stromal cells, but do not differentiate into osteocytes, under conditions where “classical” mesenchymal stem cells (MSC) would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where mesenchymal stem cells would differentiate into osteocytes or adipocytes, respectively. The MSC used for comparison in these assays are, in some embodiments, MSC that have been harvested from bone marrow (BM) and cultured under 2D conditions. In other embodiments, the MSC used for comparison have been harvested from bone marrow (BM) and cultured under 2D conditions, followed by 3D conditions.

Unless indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (Falcon, Becton, Dickinson, San Jose, Calif.) with washing medium. In some embodiments, the tissue treatment includes use of a DNAse, a non-limiting example of which is Benzonase from Merck.

Placental cells may be obtained, in certain embodiments, from a full-term or pre-term placenta. “Full-term” placenta in this regard refers to a placenta whose gestational age is at least 36 weeks. In some embodiments, residual blood is removed from the placenta before cell harvest. This may be done by a variety of methods known to those skilled in the art, for example by perfusion. In this context, the term “perfuse” or “perfusion” refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth); however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to the surface of an adherent material to thereby isolate adherent cells. In some embodiments, the donor is 35 years old or younger, while in other embodiments, the donor may be any woman of childbearing age.

Placenta-derived cells can be propagated, in some embodiments, by using a combination of 2D and 3D culturing conditions. Conditions for propagating adherent cells in 2D and 3D culture are further described hereinbelow and in the Examples section which follows.

Those skilled in the art will appreciate in light of the present disclosure that cells may be, in some embodiments, extracted from a placenta, for example using physical and/or enzymatic tissue disruption, followed by marker-based cell sorting, and then may be subjected to the culturing methods described herein.

In other embodiments, the cells are a placental cell population that does not contain a detectable amount of fetal cells and is thus entirely maternal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the cells are a placental cell population that is a mixture of fetal-derived placental ASC (also referred to herein as “fetal ASC” or “fetal cells”) and maternal-derived placental ASC (also referred to herein as “maternal ASC” or “maternal cells”), where a majority of the cells are maternal cells. In certain embodiments, the mixture is predominantly maternal cells. In more specific embodiments, the mixture contains at least 80%; at least 81%; at least 82%; at least 83%; at least 84%; at least 85%; at least 86%; at least 87%; at least 88%; at least 89%; at least 90%; at least 91%; at least 92%; at least 93%; at least 94%; at least 95%; at least 96%; at least 97%; at least 98%; at least 99%; at least 99.1%; at least 99.2%; at least 99.3%; at least 99.4%; at least 99.5%; at least 99.6%; at least 99.7%; at least 99.8%; at least 99.9%; at least 99.92%; at least 99.95%; at least 99.96%; at least 99.97%; at least 99.98%; or at least 99.99% maternal cells; or contains between 90-99%; 91-99%; 92-99%; 93-99%; 94-99%; 95-99%; 96-99%; 97-99%; 98-99%; 90-99.5%; 91-99.5%; 92-99.5%; 93-99.5%; 94-99.5%; 95-99.5%; 96-99.5%; 97-99.5%; 98-99.5%; 90-99.9%; 91-99.9%; 92-99.9%; 93-99.9%; 94-99.9%; 95-99.9%; 96-99.9%; 97-99.9%; 98-99.9%; 99-99.9%; 99.2-99.9%; 99.5-99.9%; 99.6-99.9%; 99.7-99.9%; or 99.8-99.9% maternal cells.

In still other embodiments, the preparation is a placental cell population that is a mixture of fetal and maternal cells, where a majority of the cells are fetal cells. In more specific embodiments, the mixture contains at least 70%; at least 71%; at least 72%; at least 73%; at least 74%; at least 75%; at least 76%; at least 77%; at least 78%; at least 79%; at least 80%; at least 81%; at least 82%; at least 83%; at least 84%; at least 85%; at least 86%; at least 87%; at least 88%; at least 89%; at least 90%; at least 91%; at least 92%; at least 93%; at least 94%; at least 95%; at least 96%; at least 97%; at least 98%; at least 99%; at least 99.1%; at least 99.2%; at least 99.3%; at least 99.4%; at least 99.5%; at least 99.6%; at least 99.7%; at least 99.8%; at least 99.9%; at least 99.92%; at least 99.95%; at least 99.96%; at least 99.97%; at least 99.98%; or at least 99.99% fetal cells; or contains between 90-99%; 91-99%; 92-99%; 93-99%; 94-99%; 95-99%; 96-99%; 97-99%; 98-99%; 90-99.5%; 91-99.5%; 92-99.5%; 93-99.5%; 94-99.5%; 95-99.5%; 96-99.5%; 97-99.5%; 98-99.5%; 90-99.9%; 91-99.9%; 92-99.9%; 93-99.9%; 94-99.9%; 95-99.9%; 96-99.9%; 97-99.9%; 98-99.9%; 99-99.9%; 99.2-99.9%; 99.5-99.9%; 99.6-99.9%; 99.7-99.9%; or 99.8-99.9% fetal cells.

In other embodiments, the cells are a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

Predominantly or completely maternal cell preparations may be obtained by methods known to those skilled in the art, including the protocols detailed in Example 1 of PCT Publ. Nos. WO 2016/098061, in the name of Esther Lukasiewicz Hagai et al, published on Jun. 23, 2016; and in WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus), and expanding the cells.

In other embodiments, the ASC are derived from adipose tissue. As used herein, the phrase “adipose tissue” refers to a connective tissue that comprises fat cells (adipocytes). Adipose tissue-derived ASC may be extracted, in various embodiments, by a variety of methods known to those skilled in the art, for example those described in U.S. Pat. No. 6,153,432, which is incorporated herein by reference. The adipose tissue may be derived, in other embodiments, from omental/visceral, mammary, gonadal, or other adipose tissue sites. In some embodiments, the adipose can be isolated by liposuction.

In other embodiments, ASC may be derived from adipose tissue by treating the tissue with a digestive enzyme (non-limiting examples of which are collagenase, trypsin, dispase, hyaluronidase or DNAse); and ethylenediaminetetra-acetic acid (EDTA). The cells may be, in some embodiments, subjected to physical disruption, for example using a nylon or cheesecloth mesh filter. In other embodiments, the cells are subjected to differential centrifugation directly in media or over a Ficoll or Percoll or other particulate gradient (see U.S. Pat. No. 7,078,230, which is incorporated herein by reference).

In still other embodiments, the ASC are derived from bone marrow; peripheral blood; umbilical cord blood; synovial fluid; synovial membranes; spleen; thymus; mucosa (for example nasal mucosa); limbal stroma; ligaments, for example the periodontal ligament; scalp; hair follicles, testicles; embryonic yolk sac; and amniotic fluid. In some embodiments, the ASC are human ASC, while in other embodiments, they may be animal ASC.

Surface Markers and Additional Characteristics of ASC

In some embodiments, the ASC express some or all of the following markers: CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). In some embodiments, the ASC do not express some or all of the following markers: CD3 (e.g. UniProtKB Accession Nos. P09693 [gamma chain] P04234 [delta chain], P07766 [epsilon chain], and P20963 [zeta chain]), CD4 (UniProtKB Accession No. P01730), CD11b (UniProtKB Accession No. P11215), CD14 (UniProtKB Accession No. P08571), CD19 (UniProtKB Accession No. P15391), and/or CD34 (UniProtKB Accession No. P28906). In more specific embodiments, the ASC also lack expression of CD5 (UniProtKB Accession No. P06127), CD20 (UniProtKB Accession No. P11836), CD45 (UniProtKB Accession No. P08575), CD79-alpha (UniProtKB Accession No. B5QTD1), CD80 (UniProtKB Accession No. P33681), and/or HLA-DR (e.g. UniProtKB Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the ASC possess a marker phenotype that is distinct from bone marrow-mesenchymal stem cells (BM-MSC). In certain embodiments, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g. at least 20%) of a population of cells express a marker of interest, and a significant percentage do not express the marker.

In certain embodiments, over 90% of the ASC are positive for CD29, CD90, and CD54. In other embodiments, over 85% of the described cells are positive for CD29, CD73, CD90, and CD105. In yet other embodiments, less than 3% of the described cells are positive for CD14, CD19, CD31, CD34, CD39, CD45RA (an isotype of CD45), HLA-DR, Glycophorin A, and CD200; less than 6% of the cells are positive for GlyA; and less than 20% of the cells are positive for SSEA4. In more specific embodiments, over 90% of the described cells are positive for CD29, CD90, and CD54; and over 85% of the cells are positive for CD73 and CD105. In still other embodiments, over 90% of the described cells are positive for CD29, CD90, and CD54; over 85% of the cells are positive for CD73 and CD105; less than 6% of the cells are positive for CD14, CD19, CD31, CD34, CD39, CD45RA, HLA-DR, GlyA, CD200, and GlyA; and less than 20% of the cells are positive for SSEA4. The aforementioned, non-limiting marker expression patterns were found in certain maternal placental cell populations that were expanded on 3D substrates.

In other embodiments, each of CD73, CD29, and CD105 is expressed by more than 90% of the ASC; and the cells do not differentiate into adipocytes, under conditions where mesenchymal stem cells would differentiate into adipocytes. In some embodiments, as provided herein, the conditions are incubation of adipogenesis induction medium, for example a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days. In yet other embodiments, each of CD34, CD45, CD19, CD14 and HLA-DR is expressed by less than 3% of the cells; and the cells do not differentiate into adipocytes, after incubation under the aforementioned conditions. In other embodiments, each of CD73, CD29, and CD105 is expressed by more than 90% of the cells, each of CD34, CD45, CD19, CD14 and HLA-DR is expressed by less than 3% of the cells; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days. The aforementioned solutions will typically contain cell culture medium such as DMEM+10% serum or the like, as will be appreciated by those skilled in the art. The aforementioned, non-limiting phenotypes and marker expression patterns were found in certain maternal placental cell populations that were expanded on 3D substrates.

“Positive” expression of a marker indicates a value higher than the range of the main peak of a fluorescence-activated cell sorting (FACS) isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

In still other embodiments, the majority, in other embodiments over 60%, over 70%, over 80%, or over 90% of the expanded cells express CD29, CD73, CD90, and CD105. In yet other embodiments, less than 20%, 15%, or 10% of the described cells express CD3, CD4, CD34, CD39, and CD106. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, more than 50% of the cells express, or in other embodiments highly express, CD141 (thrombomodulin; UniProt Accession No. P07204), or in other embodiments SSEA4 (stage-specific embryonic antigen 4, an epitope of ganglioside GL-7 (IV³ NeuAc 2→3 GalGB4); Kannagi R et al), or in other embodiments both markers. Alternatively or in addition, more than 50% of the cells express HLA-A2 (UniProt Accession No. P01892). The aforementioned, non-limiting marker expression patterns were found in certain fetally-derived placental cell populations that were expanded on 3D substrates. The Uniprot Accession Nos. mentioned in the paragraph were accessed on accessed on Feb. 8, 2017.

In other embodiments, each of CD29, CD73, CD90, and CD105 is expressed by more than 80% of the cells that have been expanded; and the cells do not differentiate into osteocytes, after incubation for 17 days with a solution containing 0.1 mcM dexamethasone, 0.2 mM ascorbic acid, and 10 mM glycerol-2-phosphate, in plates coated with vitronectin and collagen. In yet other embodiments, each of CD34, CD39, and CD106 is expressed by less than 10% of the cells; less than 20% of the cells highly express CD56; and the cells do not differentiate into osteocytes, after incubation under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the aforementioned conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen. The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

In other embodiments, each of CD29, CD73, CD90, and CD105 is expressed by more than 80% of the cells that have been expanded; and the cells do not differentiate into adipocytes, after incubation in adipogenesis induction medium, namely a solution containing 1 mcM dexamethasone, 0.5 mM IBMX, 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days. In yet other embodiments, each of CD34, CD39, and CD106 is expressed by less than 10% of the cells; less than 20% of the cells highly express CD56; and the cells do not differentiate into adipocytes, after incubation under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days. The aforementioned solutions will typically contain cell culture medium such as DMEM+10% serum or the like, as will be appreciated by those skilled in the art. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

Additionally or alternatively, the ASC secrete or express (as appropriate in each case) IL-6 (UniProt identifier P05231), IL-8 (UniProt identifier P10145), eukaryotic translation elongation factor 2 (EEEF2), reticulocalbin 3, EF-hand calcium binding domain (RCN₂), and/or calponin 1 basic smooth muscle (CNN1). In more specific embodiments, greater than 50%, in other embodiments greater than 55%, in other embodiments greater than 60%, in other embodiments greater than 65%, in other embodiments greater than 70%, in other embodiments greater than 75%, in other embodiments greater than 80%, in other embodiments greater than 85%, in other embodiments greater than 90%, in other embodiments greater than 95%, in other embodiments greater than 96%, in other embodiments greater than 97%, in other embodiments greater than 98%, in other embodiments greater than 99%, of the cells express or secrete at least one, in other embodiments at least 2, in other embodiments at least 3, in other embodiments at least 4, in other embodiments all five of the aforementioned proteins.

Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% fetal bovine serum (FBS)+2 mM L-Glutamine), added to each well of a 6 well-plate, and cultured for 24 hrs in a humidified incubator (5% CO₂, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

In still other embodiments, the ASC secrete Flt-3 ligand (Fms-related tyrosine kinase 3 ligand; Uniprot Accession No. P49772), stem cell factor (SCF; Uniprot Accession No. P21583), IL-6 (Interleukin-6; UniProt identifier P05231), or combinations thereof, each of which represents a separate embodiment. In certain embodiments, the ASC secrete levels of Flt-3 ligand, SCF, IL-6, or in other embodiments combinations thereof, that are at least 2-, 3-, 4-, 5-, 6-, 8-, 10-, 12-, 15-, or 20-fold higher than that expressed or secreted by ASC of placenta tissue grown on a 2D substrate. ASC grown on a 3D substrate secrete higher levels of Flt-3 ligand, SCF, and IL-6 than ASC grown on a 2D substrate, as provided in PCT Application Publ. No. WO/2007/108003, which is fully incorporated herein by reference in its entirety. Uniprot entries in this and the following 2 paragraphs were accessed on Feb. 26, 2017.

In other embodiments, the described ASC exhibit a spindle shape when cultured under 2D conditions.

In still other embodiments, the population of ASC is positive (on a population level) for expression of CD10 (neprilysin; UniProtKB Accession No. P08473), CD29, CD38 (ADP-ribosyl cyclase; UniProtKB Accession No. P28907), and CD40 (UniProtKB Accession No. P25942). Optionally, the majority of the cells also express CD90. Alternatively or in combination, the majority of the cells also express one or more, in other embodiments 2 or more, in other embodiments 3 or more, in other embodiments all 4 of: CD74 (HLA class II histocompatibility antigen gamma chain; UniProtKB Accession No. P04233), CD106 (Vascular cell adhesion protein 1 [VCAM]; UniProtKB Accession No. P19320), CD274 (Programmed cell death 1 ligand 1; UniProtKB Accession No. Q9NZQ7), and HLA-DR. Positivity for marker expression “on a population level” as used herein means that expression of each of the indicated markers is above the indicated threshold level for that particular marker. Alternatively or in combination, the population is at least 40% positive on a population level for one or more, in other embodiments 2 or more, in other embodiments 3 or more, in other embodiments 4 or more, in other embodiments all 5 of: CD42a (Platelet glycoprotein IX; UniProtKB Accession No. P14770), CD45Ra (an isotype of CD45 [Protein tyrosine phosphatase, receptor type, C]; UniProtKB Accession No. P08575), CD77 (Lactosylceramide 4-alpha-galactosyltransferase; UniProtKB Accession No. Q9NPC4), CD243 (Multidrug resistance protein 1; UniProtKB Accession No. P08183), and CD275 (ICOS ligand; UniProtKB Accession No. O75144). In further embodiments, at least 40% of the population is negative for expression of CD9 (UniProtKB Accession No. P21926). In certain embodiments, the population of cells is derived from placental tissue. All UniProtKB entries mentioned in this paragraph were accessed on Jan. 22, 2015. In certain embodiments, the cells express (and/or lack, as indicated above) one of the aforementioned combinations of markers and do not differentiate into osteocytes, under conditions where “classical” MSC would differentiate into osteocytes, as described herein. In other embodiments, the cells express (and/or lack) one of the aforementioned combinations of markers and do not differentiate into adipocytes, under conditions where MSC would differentiate into adipocytes, as described herein. In still other embodiments, the cells express (and/or lack) one of the aforementioned combinations of markers and do not differentiate into either osteocytes or adipocytes, under conditions where MSC would differentiate into osteocytes or adipocytes, respectively.

According to some embodiments, the ASC express CD200, while in other embodiments, the ASC lack expression of CD200. In still other embodiments, less than 30%, 25%, 20%, 15%, 10%, 8%, 6%, 5%, 4%, 3%, or 2%, 1%, or 0.5% of the adherent cells express CD200. In yet other embodiments, greater than 70%, 75%, 80%, 85%, 90%, 92%, 94%, 95%, 96%, 97%, 98%, 99%, or 99.5% of the adherent cells express CD200.

In still other embodiments, the cells may be allogeneic, or in other embodiments, the cells may be autologous. In other embodiments, the cells may be fresh or, in other embodiments, frozen (e.g., cryo-preserved).

In still other embodiments, the ASC (“induced ASC”) have any of the aforementioned characteristics, or in other embodiments any combination thereof, after they have undergone induction. Each characteristic represents a separate embodiment.

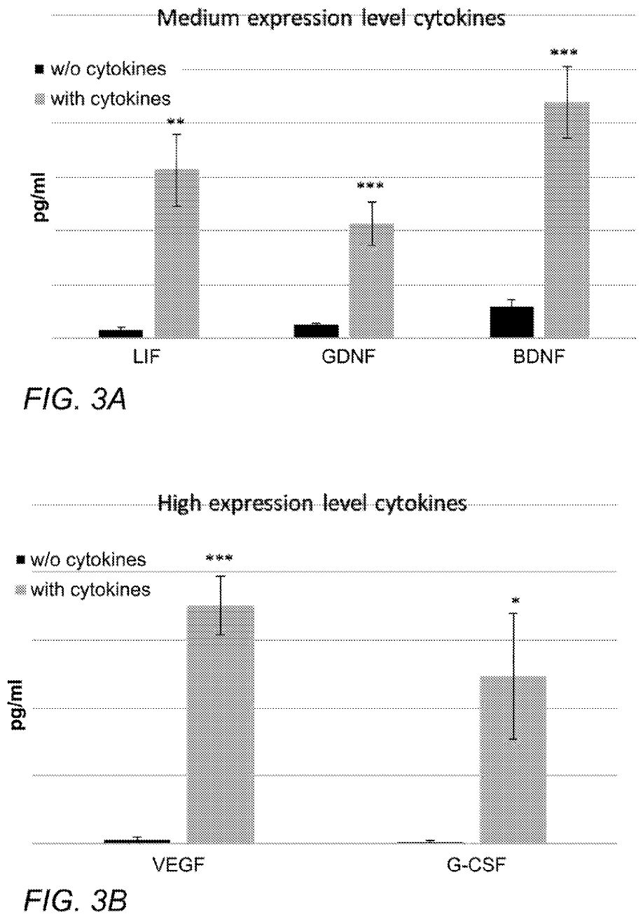

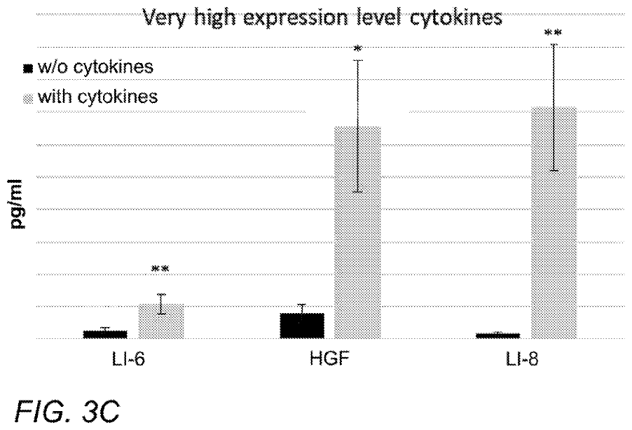

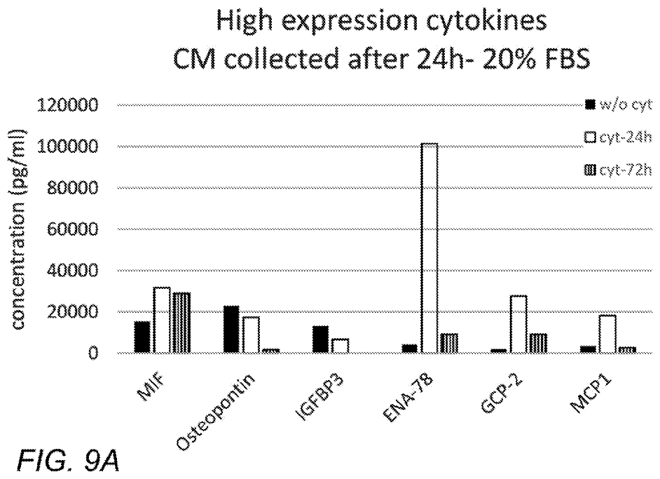

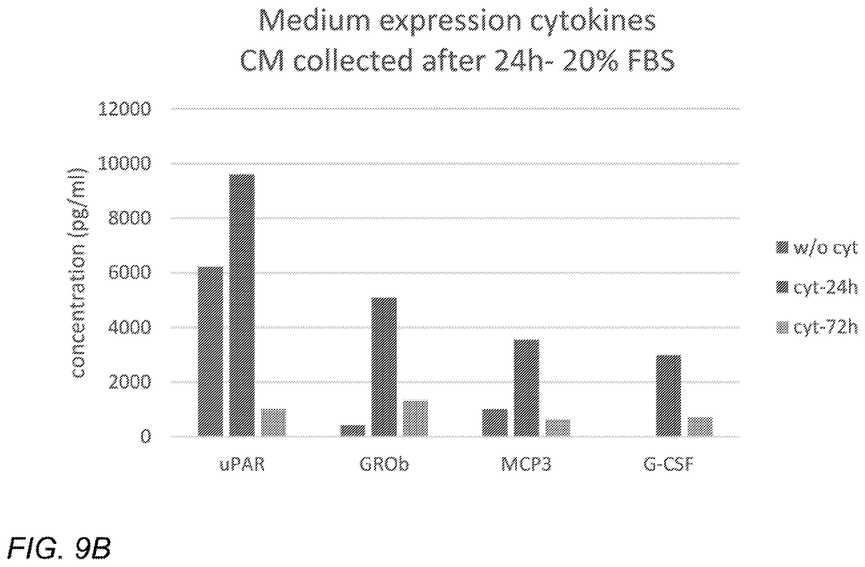

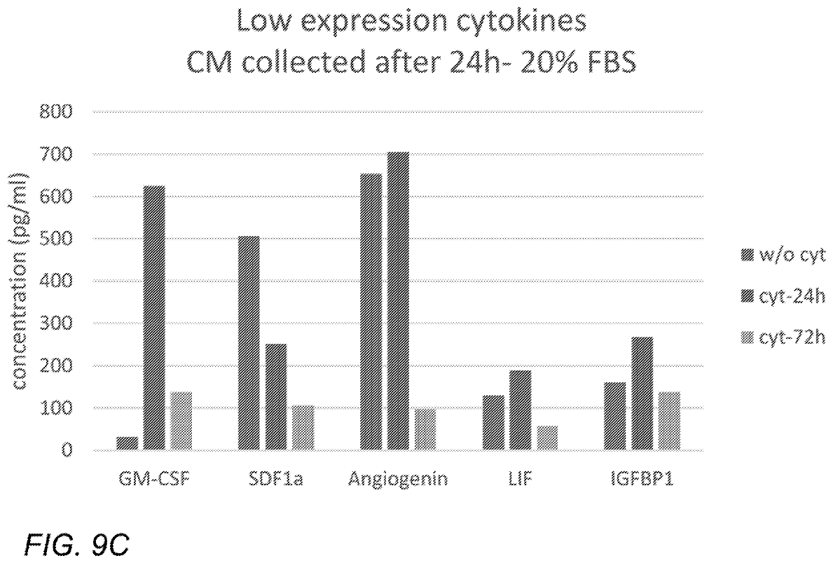

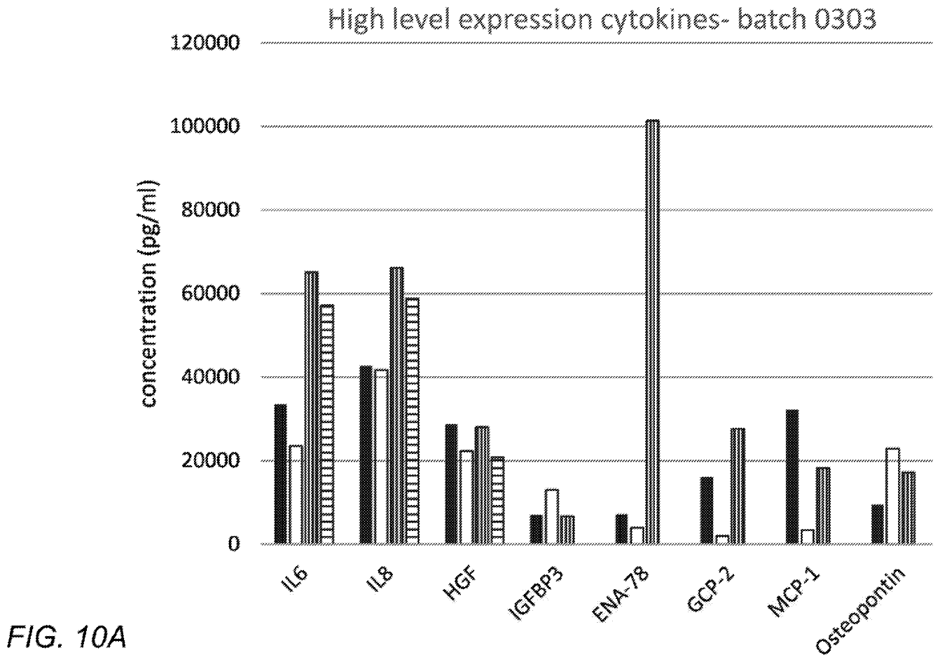

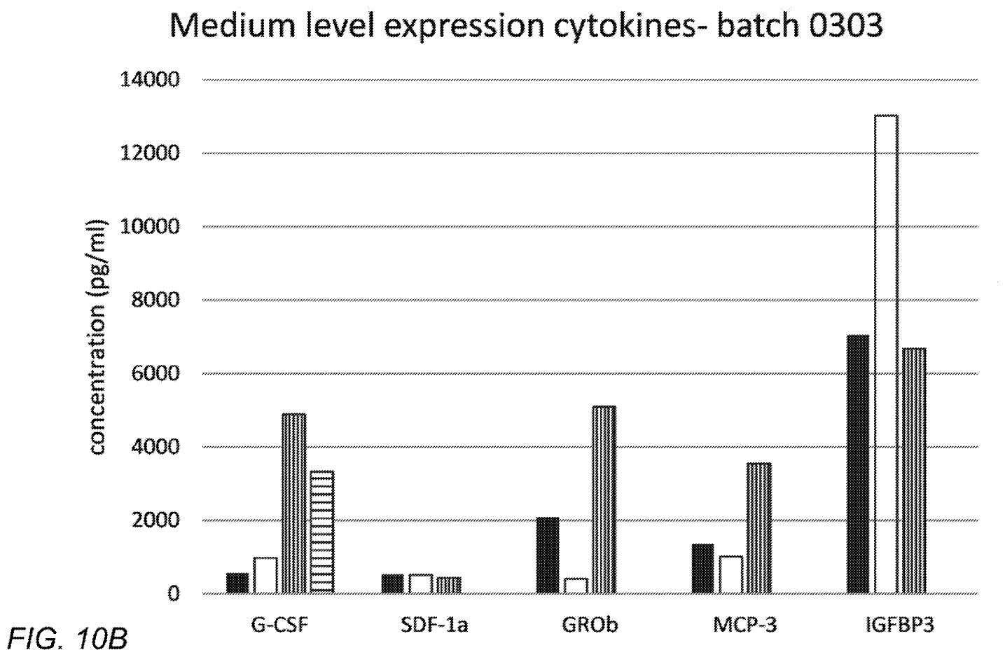

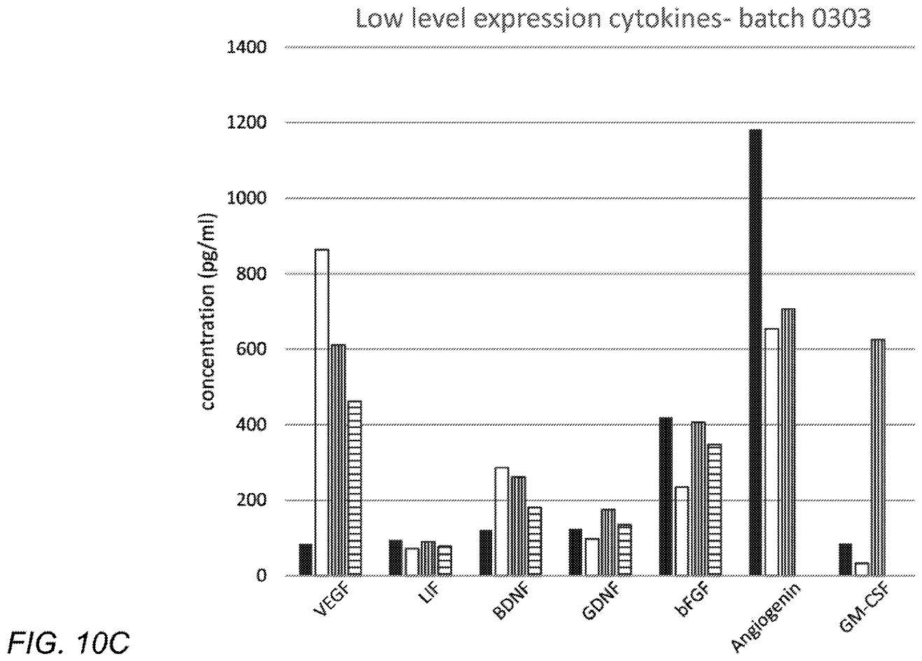

Alternatively or in addition, the induced ASC secrete over 200 pg/ml BDNF, when 2×10\hat{ }5 cells (following induction and optionally cryopreservation) are seeded in 6-well plates, in 2 ml DMEM+10% FBS medium, followed by incubation in serum-free DMEM for 72 hours and measurement of BDNF in the CM. In other embodiments, under the same conditions, the induced ASC secrete over 250 pg/ml BDNF; over 300 pg/ml BDNF; over 400 pg/ml BDNF; over 500 pg/ml BDNF; over 600 pg/ml BDNF; over 800 pg/ml BDNF; over 1000 pg/ml BDNF; over 1200 pg/ml BDNF; over 1500 pg/ml BDNF; or over 1800 pg/ml BDNF (Example 3). In other embodiments, the ASC secrete over 2000 pg/ml BDNF; over 2500 pg/ml BDNF; over 3000 pg/ml BDNF; over 4000 pg/ml BDNF; over 5000 pg/ml BDNF; over 6000 pg/ml BDNF; or over 7000 pg/ml BDNF, when the CM is produced in DMEM+20% FBS (Example 7). In other embodiments, the induced ASC secrete over 1000; over 1200; over 1500; over 2000; over 2500; over 3000; or over 3500 pg BDNF per 10\hat{ }6 cells into the induction medium itself (Example 7). In other embodiments, the aforementioned amounts of BDNF are secreted in the first, the second, or the third 24-hour period of incubation in serum-free DMEM (Example 4). In still other embodiments, the induced ASC secrete any of the other factors shown in FIGS. 3A-C in the indicated amount, or with less than a 2-fold difference in the amount. In still other embodiments, the induced ASC secrete any of the other factors shown in FIG. 9A-C, in the indicated amount, or with less than a 2-fold difference in the amount, after directly incubating induced, cryopreserved cells for 24 hr. in DMEM+20% FBS (Example 7). In other embodiments, the induced ASC secrete any of the other factors shown in FIGS. 10A-C into the induction medium in the indicated amount, or with less than a 2-fold difference in the amount, during the last 24 hours of induction (Example 8). Each factor represents a separate embodiment of the present invention.

In certain embodiments, further steps of purification or enrichment for ASC may be performed. Such methods include, but are not limited to, cell sorting using markers for ASC and/or, in various embodiments, mesenchymal stromal cells or mesenchymal-like stromal cells. Typically, these further steps are performed prior to induction.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

Therapeutic Methods and Compositions

Thus, in certain embodiments is provided a method of treating a neurodegenerative disease in a subject in need thereof, comprising the step of administering to the subject a pharmaceutical composition comprising induced ASC, thereby treating a neurodegenerative disease. Also provided is a composition for treating a neurodegenerative disease, comprising induced ASC. Also provided is use of induced ASC for the manufacture of a medicament for treating a neurodegenerative disease. In certain, more specific embodiments, the treated neurodegenerative disease is an ataxia. In other embodiments, the disease is a dementia. In still other embodiments, the disease is Alzheimer's disease; is Parkinson's disease; is Amyotrophic lateral sclerosis (ALS); is Huntington's disease; is SMA, each of which represents a separate embodiment. In still other embodiments, the treated neurological disease is MS, or is ataxia-telangiectasia

Thus, in certain embodiments is provided a method of inhibiting the development of a neurodegenerative disease in a subject in need thereof, comprising the step of administering to the subject a pharmaceutical composition comprising induced ASC, thereby inhibiting the development of a neurodegenerative disease. Also provided is a composition for inhibiting the development of a neurodegenerative disease, comprising induced ASC. Also provided is use of induced ASC for the manufacture of a medicament for inhibiting the development of a neurodegenerative disease. In certain, more specific embodiments, the neurodegenerative disease is Alzheimer's disease; is Parkinson's disease; is ALS; is Huntington's disease; is SMA, each of which represents a separate embodiment. In still other embodiments, the neurological disease is MS, or is ataxia-telangiectasia

In various embodiments, the described cells are able to exert the described therapeutic effects, each of which is considered a separate embodiment, with or without the cells themselves engrafting in the host. For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days. In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

In other embodiments, conditioned medium (CM) secreted by the described induced cells is used in place of induced ASC in the described methods and compositions. Also provided are pharmaceutical compositions, comprising the described CM. Those skilled in the art will appreciate that, in certain embodiments, various bioreactors may be used to prepare CM, including but not limited to plug-flow bioreactors, and stationary-bed bioreactors (Kompier R et al. Use of a stationary bed reactor and serum-free medium for the production of recombinant proteins in insect cells. Enzyme Microb Technol. 1991. 13(10): 822-7.)

In yet other embodiments, exosomes secreted by the described induced cells are used in the described methods and compositions. Methods of isolating exosomes are well known in the art, and include, for example, immuno-magnetic isolation, for example as described in Clayton A et al, 2001; Mathias R A et al, 2009; and Crescitelli R et al, 2013. Provided in addition are pharmaceutical compositions, comprising the described ASC. In some embodiments, the exosomes are harvested from a 3D bioreactor in which the induced cells have been incubated. Alternatively or in addition, the cells are cryopreserved, and then are thawed, after which the exosomes are isolated. In some embodiments, after thawing, the exosomes are cultured on a 2D substrate, from which the exosomes are harvested.

The described ASC or CM, derived therefrom, is, in certain embodiments, administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprise ASC or CM in combination with an excipient, e.g., a pharmacologically acceptable excipient. In further embodiments, ASC are provided with excipient is an osmoprotectant or cryoprotectant, or is a carrier protein. In still further embodiments, the described osmoprotectant or cryoprotectant protects cells from the damaging effect of freezing and ice formation. In more specific embodiments, the osmoprotectant or cryoprotectant is a permeating compound, non-limiting examples of which are dimethyl sulfoxide (DMSO), glycerol, ethylene glycol, formamide, propanediol, poly-ethylene glycol, acetamide, propylene glycol, and adonitol; or is in other embodiments a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and carrier protein are present; in certain embodiments, the osmoprotectant and carrier protein may be the same compound. Alternatively or in addition, the composition is frozen. In certain embodiments, the cells are washed after thawing, e.g. to remove or minimize excipients such as DMSO, which may be harmful, in certain embodiments, to the central nervous system, particularly when cells are administered intracerebrally, by intracerebroventricular administration, intrathecally, or intranasally. The cells may be any embodiment of induced ASC mentioned herein, each of which is considered a separate embodiment.

Since non-autologous cells may in some cases induce an immune reaction when administered to a subject, several approaches may be utilized according to the methods provided herein to reduce the likelihood of rejection of non-autologous cells. In some embodiments, these approaches include either suppressing the recipient immune system or encapsulating the non-autologous cells in immune-isolating, semipermeable membranes before transplantation. In some embodiments, this may be done whether or not the ASC themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days.

Examples of immunosuppressive agents that may be used in the methods and compositions provided herein include, but are not limited to, methotrexate, cyclophosphamide, cyclosporine, cyclosporine A, chloroquine, hydroxychloroquine, sulfasalazine (sulphasalazopyrine), gold salts, D-penicillamine, leflunomide, azathioprine, anakinra, infliximab (REMICADE), etanercept, TNF-alpha blockers, biological agents that antagonize one or more inflammatory cytokines, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, and tramadol.

One may, in various embodiments, administer the pharmaceutical composition in a systemic manner. Alternatively, one may administer the pharmaceutical composition locally, for example, via injection of the pharmaceutical composition directly into an affected tissue region of a patient. In other embodiments, the cells are administered intracerebrally, by intracerebroventricular administration, intrathecally, or intranasally, each of which is considered a separate embodiment. In still other embodiments, cells are administered subcutaneously, intramuscularly, intravenously, or intraperitoneally. In certain embodiments, damage to the blood-brain barrier, as may be observed in neurodegenerative diseases, enables the described cells to cross the blood-brain barrier when administered systemically, i.e. intravenously.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from several days to several weeks or, in other embodiments, until alleviation of the disease state is achieved.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The described induced cells are, in some embodiments, suitably formulated as pharmaceutical compositions which can be suitably packaged as an article of manufacture. Such an article of manufacture comprises a packaging material which comprises a label describing a use in treating a disease or disorder that is mentioned herein. In other embodiments, a pharmaceutical agent is contained within the packaging material, wherein the pharmaceutical agent is effective for the treatment of an immune-mediated or circulatory disorder. In some embodiments, the pharmaceutical composition is frozen.

A typical dosage of the described induced cells for a human subject ranges, in some embodiments, from about 10 million to about 1,000 million cells, about 10-500 million cells; or about 50-500 million cells per administration. For example, the dosage can be, in some embodiments, 10, 20, 30, 40, 50, 60, 70, 80, 90, 100, 125, 150, 175, 200, 225, 250, 275, 300, 325, 350, 375, 400, 425, 450, 475, or 500 million cells or any amount in between these numbers. It is further understood that a range of ASC can be used including from about 10 to about 500 million cells, from about 100 to about 400 million cells, from about 150 to about 300 million cells. Accordingly, disclosed herein are therapeutic methods, the method comprising administering to a subject a therapeutically or prophylactically effective amount of ASC, wherein the dosage administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or, in other embodiments, between 150 million to 300 million cells. ASC, compositions comprising ASC, and/or medicaments manufactured using ASC can be administered, in various embodiments, in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, or 5-50 injections, or more.

In various embodiments, the ASC are administered to the subject multiple times, over the course of at least 1 month, at least 2 months, at least 3 months, at least 4 months, at least 5 months, at least 6 months between 1-24 months, between 2-24 months, between 3-24 months, between 4-24 months, between 5-24 months, between 6-24 months, between 1-12 months, between 2-12 months, between 3-12 months, between 4-12 months, between 5-12 months, or between 6-12 months, following diagnosis of a neurodegenerative disease, which may be, in various, more specific embodiments, Alzheimer's disease, Parkinson's disease, Amyotrophic lateral sclerosis (ALS), Huntington's disease, or SMA, each of which represents a separate embodiment. In still other embodiments, the neurological disease is MS, or is ataxia-telangiectasia.

It is clarified that each embodiment of the described induced cells may be freely combined with each embodiment relating to a therapeutic method or pharmaceutical composition.

Furthermore, each embodiment of the described exosomes may be freely combined with each embodiment relating to a therapeutic method or pharmaceutical composition.

Additionally, each embodiment of conditioned medium may be freely combined with each embodiment relating to a therapeutic method or pharmaceutical composition.

### Subjects

In certain embodiments, the subject treated by the described methods and compositions is a human subject having a neurological disorder. Alternatively or in addition, the human is elderly (e.g. over age 65). In other embodiments, the human is a age 1-18, 18-30, 30-40, 40-50, 50-60, or over age 60. In other embodiments, the subject is an animal. In other embodiments, the subject is an animal subject having a neurological disorder. In some embodiments, treated animals include domesticated animals and laboratory animals, e.g., non-mammals and mammals, for example non-human primates, rodents, pigs, dogs, and cats. Alternatively or in addition, the subject may be administered with additional therapeutic agents or cells.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including adherent stromal cells. In another aspect, the kits and articles of manufacture may comprise a label, instructions, and packaging material, for example for treating an immune-mediated or circulatory disorder or for other therapeutic indications mentioned herein.

Those skilled in the art will appreciate that a competent physician is capable of diagnosing and following Alzheimer's disease, Parkinson's disease, ALS, Huntington's disease, MS, SMA, and ataxia-telangiectasia, in each particular circumstance.

Also provided herein, in various embodiments, is a method of reducing an incidence of a neurological and/or neurodegenerative disease, for example Alzheimer's disease, Parkinson's disease, ALS, Huntington's disease, MS, SMA, or ataxia-telangiectasia. Also provided is a composition for reducing an incidence of a neurodegenerative disease, comprising induced ASC. Also provided is use of induced ASC for the manufacture of a medicament for reducing an incidence of a neurodegenerative disease

As provided herein, induced ASC stimulated the differentiation of SH-SY5Y cells, as evidenced by a significant morphological change, including the appearance of long neurites extending from the cells. The human neuroblastoma derived cell line SH-SY5Y is an undifferentiated line of cell that continuously proliferate and express immature neuronal markers, but lack mature neuronal markers. These cells are considered to be most reminiscent of immature catecholaminergic neurons. Following treatment with differentiation-inducing agents, SH-SY5Y cells become morphologically more similar to primary neurons with long, exquisite processes (Neuronal Cell Culture. [Humana Press, Eds. Shohreh Amini and Martyn K. White, copyright Springer Science 2013]), and have the potential to differentiate into either cholinergic, dopaminergic or noradrenergic phenotypes, depending on medium conditions. These cells are therefore a good model to test the potential of induced ASC to elicit neuronal differentiation of neural precursor cells.

Methods for testing therapeutics for neurodegenerative diseases, for example Alzheimer's, are well known in the art, and include, for example, the SAMP8 mouse model (Takeda Industries, Japan), which is accepted as a model to study the interactions between overproduction of Aβ and oxidative damage to brain tissue. SAMP8 mice have a spontaneous mutation resulting in the overproduction of amyloid precursor protein (APP) and oxidative damage. By 8-10 months of age, the animals develop deficits in learning and memory, together with an age related increase in Aβ, tau phosphorylation and oxidative stress. Amyloid plaques occur later in life (˜17 months). Studies using a variety of techniques have demonstrated that both learning and memory deficits in the SAMP8 mice can be reversed by multiple pharmacological agents that modulate the neurotransmitters implicated in AD (Flood et al, 1993; Flood et al, 1996; Flood et al, 1998). Furthermore, this mouse model is characterized by neuronal cell death, a central feature of AD that is not recapitulated in the most frequently used transgenic mouse models of AD (which display overexpression of Aβ and/or phosphorylated tau) (Morley et al). The main characteristics of SAMP8 mice and a comparison with the most frequently used transgenic mouse models of AD are summarized in Table 1.

TABLE 1
Comparison of Alzheimer's disease,
SAMP8 mouse and transgenic mice models.

| | Alzheimer's | | Transgenic |
|---|---|---|---|
| | disease | SAMP8 | models |
| Overproduction of amyloid-β | Yes | Yes | Yes |
| Amyloid plaques | Yes | Late* | Yes |
| Phosphorylated tau | Increased | Increased | In some models |
| Cerebral amyloid angiopathy | Yes | Yes | Yes |
| Neuron loss | Yes | Yes | ? |
| Synaptic dysfunction | Yes | Yes | Yes |
| Dendritic spine loss | Yes | Marked | ? |
| Gliosis | Yes | Yes | Yes |
| Cholinergic deficit | Yes | Yes | Yes |
| Learning and memory impaired | Yes | Yes | Yes |
| Circadian rhythm disturbances | Yes | Yes | ? |
| Oxidative damage | Yes | 4 months | 8 months |

? = uncertain.
*Occur at 16 to 18 months.

Additional Alzheimer's disease models include, for example, the models described herein and the other models described in Holm et al, and the references cited therein.

Parkinson's disease models are well known in the art and include, for example, the 6-hydroxydopamine (6-OHDA) model and the other models described in Naughton et al, Panicker et al, Holm et al, and the references cited in these publications. In various embodiments, ASC may be induced or non-induced.

ALS models are well known in the art and include, for example, various mSOD1 models, a non-limiting example of which is the G93A mutant, and L-BMAA models, and the other models described in Kunis et al, de Pedro et al, Holm et al and the references cited in these publications. Testing for ALS with ASC (induced or non-induced, in various embodiments) may include, in various embodiments, initiation of treatment is either before the onset of motor deficits (non-limiting examples of which are weakness in 1 or both hind limbs or paralysis of 1 or both hind limbs) or shortly after the first observation of motor deficits and/or weight loss. In certain embodiments, the weight loss threshold for initiation of treatment is 5%, 10%, or 15% of the peak weight. Treatment can be, in various embodiments, a single administration or periodic (e.g. weekly) administrations until death or termination of the study.

Huntington's disease models are well known in the art and include, for example, the pig models described in Holm et al, Yang et al, and the references cited in these publications. In various embodiments, ASC may be induced or non-induced.

Animal models of MS are well known in the art and include, for example, the B cell-dependent and T cell-mediated models described in Hausler et al and Fan et al, the genetic models described in Marino et al, the experimental autoimmune encephalomyelitis (EAE) mice described in Magliozzi et al, and the references cited in these publications. In various embodiments, ASC may be induced or non-induced.

Animal models of ataxia-telangiectasia are well known in the art and include, for example, the mouse model described in Duecker R et al and the references cited therein, and the pig models described in Holm et al and the references cited therein. In various embodiments, ASC may be induced or non-induced.

Animal models of SMA are well known in the art and include, for example, the mouse models described in Alrafiah A et al and the references cited therein, and the pig models described in Holm et al and the references cited therein. In various embodiments, ASC may be induced or non-induced.

Animal models of SCA are well known in the art, and include, for example, those described in Mieda et al and the references cited therein. In various embodiments, treatment in the model is by a single administration at 5 weeks of age or multiple administrations, e.g. 2 administrations separated by 2-4 weeks, e.g. beginning at 5 weeks.

Methods for behavioral and cognitive testing of laboratory animals are well known in the art. For example, T maze tests and passive avoidance tests (Ouhaz et al); novel object and novel place recognition tests (Mendez et al); elevated plus-mazes, open-field apparatuses, and activity meters (Mechan et al); behavioral tests (e.g. Rotarod, grasping and BBB, for assessing motor capabilities [as described herein]); and bow-tie mazes (Mathieu et al) may be used. In various embodiments, ASC may be induced or non-induced.

In other embodiments, biochemical and histochemical analysis are used to determine the effect of putative therapeutic modalities on neurodegenerative diseases. Methods for conducting such analyses, including measurement of oxidative damage and markers of Alzheimer's presence and progression, are well known to those skilled in the art.

Those skilled in the art will appreciate the role of oxidative stress (OS) in the pathogenesis and progression of Parkinson's, AD and ALS (Mathis et al). OS results from the cumulative formation of reactive oxygen species (ROS) and reactive nitrogen species (RNS) which may induce a cellular redox imbalance (Valko et al). The collateral damage is characterized by oxidative modification of a number of cellular macromolecular targets, including proteins, lipids, carbohydrates, DNA and RNA. SAMP8 mice exhibit increased oxidative damage in the brain (Morley et al, 2012; Butterfield et al 2005; Farr et al 2003), which makes this a suitable model to assess the effect of treatment on OS.

As provided herein, induced ASC are shown herein to reduce OS. Without wishing to be bound by theory, pharmacological approaches for intervening in OS may provide therapeutic intervention strategies for neurodegenerative disease.

Those skilled in the art will also appreciate that free radical-related OS causes molecular damage that can lead to a failure of biological functions, protein modification, misfolding, aggregation, and ultimately, cell death. Functional deficits of the mitochondrial function can cause a major intracellular generation of reactive oxygen species (ROS), such as superoxide and H₂O₂, resulting in increased formation of hydroxyl free radicals.

Methods for ascertaining protection from oxidative stress-induced neuronal cell death are well known in the art. For example, the cytoprotective/antioxidant effects of ASC can be tested in C2C12 myoblasts and SH-SY5Y neuroblastoma cells, using H₂O₂ as an oxidative stress inducer. Cell survival and viability of C2C12 cells can be determined using the Real Time-Glo™ method following H₂O₂-induced oxidative damage, in the presence or absence of ASC cells or cell-derived CM. A similar assay can be performed in H₂O₂-exposed C2C12 cells, using the DCF-DA assay, as exemplified herein.

The cytoprotective/antioxidant effects of ASC can also be tested in a co-culture experiment. Such experiments would have live ASC adherent to the culture apparatus and present at the time of exposure to H₂O₂.

In other embodiments, a number of parameters can be monitored, including:
Protein carbonylation—protein carbonyls are an index of protein oxidation (Farr et al, 2014) that can be detected by the 2,4-dinitrophenyl hydrazine (DNP) Schiff-base adduct.
Lipid peroxidation—protein bound 4-hydroxynonenal (HNE) is an index of lipid peroxidation (Sultana et al).
Protein nitrosylation—protein bound 3-nitrotyrosine (3-NT) is a marker of reactive nitrogen species formation.

Typically, the product may be detected using slot blot analysis and quantified by densitometry to determine whether induced ASC treatment reduces general oxidation in the brain areas examined.

AD Phenotypic Markers:

Pathologically AD is characterized by the accumulation of neurofibrillary tangles (NFT) and amyloid beta (Aβ) plaques, two primary hallmarks of the disease closely associated with cognitive decline (Clinton et al). The following are non-limiting examples of markers of Aβ and NFT accumulation:
APP—the amyloid precursor protein from which Aβ peptides are processed by β and γ secretases (Butterfield et al 1997).
Aβ—Aβ plays a pivotal role in the pathophysiology of AD through its ability to induce free radical damage to neuronal membrane components (Butterfield et al 1997).
Levels of tau phosphorylation-hyperphosphorylated tau is the main component of NFTs which are highly detrimental to neurons

Gsk-3β Levels—

Glycogen Synthase kinase 3β (GSK-3β) is a pleiotropic enzyme involved in a variety of cell activities, which has also been postulated as a therapeutic target for AD (Mondragon-Rodriguez et al). In the brain, GSK-3β is the predominant kinase that phosphorylates tau. Brains of AD subjects reportedly have increased GSK-3β, resulting in the hyperphosphorylation and related NFT generation of AD (Cho et al).

Skilled artisans will also be familiar with methods of ascertaining the effect of therapeutic modalities on mitochondrial damage in neurons and muscle cells. For example, the mitochondria-specific fluorescent dye, MitoTracker® Green/Red or MitoSOX™ can be used to assess the effect of ASC on active mitochondrial mass in C2C12 and SH-SY5Y cells after exposure to H₂O₂, which impairs mitochondrial function. Alternatively, it is possible to examine whether ASC cells are able to inhibit H₂O₂-induced mitochondrial transmembrane potential (Δψm) loss by using the MitoProbe™ JC-1. The reagents mentioned herein are available from Thermo Fisher Scientific (Waltham, Mass.).

Alternatively or in addition, histological and immunohistochemical analysis can be used to determine the effect of therapeutic modalities on neurodegenerative diseases. Methods for conducting such analyses, including measurement of microglial activation, neuronal loss, and neurovascular pathology, are well known to those skilled in the art. An exemplary, non-limiting protocol is transcardial perfusion at sacrifice in order to eliminate blood and fix brain tissue before sectioning and punching out the hippocampus and the cortex, followed by staining.

For microglia activation, in some embodiments, immunofluorescent staining for Iba-1, a general marker of microglia, and CD68, a microglial activation marker (Mosher et al) can be used, serving as an indicator of neuro-inflammation, characterized by proliferation of microglia with an active (CD68+) phenotype. Chronically-activated microglia are thought to be active contributors to neuronal damage because of excessive production of cytotoxic factors such as superoxide, nitric oxide (NO), and tumor necrosis factor (TNF-α) (Heneka et al). The number of microglia cells and their activation status can serve as a measure of inflammation.

It will also be appreciated that in ALS models (e.g. SOD1G93A mice), reduction of microgliosis and astrocytosis, modulates microglia-related inflammatory genes, and enhances motor neuron survival. Neuronal cell rescue by ASC can be evaluated histologically by determining motor neuron cell number in the ventral horn of the lumbar spinal cord. ChAT antibody can be used to visualize motor neuron cell bodies. Microglial and astrocyte activation can also be examined using the Iba-1 and GFAP antibodies, respectively. Staining is performed on the lumbar section of the spinal cord (L2-L5).

For neuronal loss, it will be appreciated that immunohistochemical analysis of the sections can be used to detect neuronal loss, a pathological marker associated with cognitive decline. It can be determined, for example, by quantifying the number of neurons on NeuN-stained slides, assessing dendritic length, and quantifying branching points with Golgi staining.

Neuro-vascular pathology may be determined, in some embodiments, by detecting microhemorrhages and studying blood vessel area after staining slides with an endothelial cell marker.

Methods of testing differentiation of neuronal cells in vitro are well known in the art, and include, for example, monitoring reduction or disappearance of expression of markers of immature neurons and appearance of mature neuron markers. Immature neuron markers include but are not limited to Proliferating cell nuclear antigen (PCNA), Nestin, and Differentiation inhibiting transcription factors (ID1,ID2,ID3). Mature neuron markers include but are not limited to Neuron specific enolase (NSE), β-III-tubulin, Microtubule associated protein-2 (MAP2), Synaptophysin, and NeuN.

Methods of determining the bio-distribution of cells are also well known in the art. For example, the location of ASC following administration to the cerebrospinal fluid can utilize anti-human-Ku80-ab, which localizes human cells.

Methods of determining the integrity of the neuro-muscular junctions (NMJ) are also well known in the art. SOD1G93A mice are characterized by NMJ denervation. The gastrocnemius (GNS) muscle can be stained with anti-α-bungarotoxin, to stain post-synaptic acetyl choline receptor clusters, while synapses of motor axons on the GNS muscle can be localized with antibodies to neurofilament (SMI-31) and synaptophysin. Muscle denervation and atrophy can be followed using Real-time PCR and ELISA to measure expression of relevant markers (AChRα and AChRγ, MuSK; and MuRF1 and Atg-1, respectively).

Methods of ascertaining muscle wasting, which likely contributes to ALS progression, are also well known in the art. The effect of ASC treatment on GNS muscle morphology in ALS-model mice can be ascertained using H&E staining to count the number of cells displaying a regular myofibril morphology or atrophy.

It will also be appreciated that Creatine Kinase (CK) is associated with skeletal muscle damage and can be used to differentiate myopathic from neurogenic lesions. Raised serum CK concentrations are found in ALS patients. Serum CK levels can be measured in ASC-vs. placebo-treated SOD1G93A mice with a Creatine Kinase Activity Assay Kit.

Induction of ASC

As provided herein, ASC can be induced by incubation with medium comprising agents that cause them to secrete neurotrophic and/or neuroprotective factors. Such additives may be referred to herein as induction agents, or simply “agents”. Examples of secreted factors are BDNF (brain derived neurotrophic factor; Uniprot Accession No. P23560), GDNF (glial cell line derived neurotrophic factor; Uniprot Accession No. P39905), bFGF (basic fibroblast growth factor; Uniprot accession no. P09038), NGF (nerve growth factor; Uniprot Accession No. P01138), VEGF (vascular endothelial growth factor; Uniprot Accession No. P156), HGF (hepatocyte growth factor; Uniprot Accession No. P08581), and LIF (Leukemia inhibitory factor; Uniprot Accession No. P15018). In certain embodiments, the secreted factors comprise one or more of BDNF, GDNF, bFGF, NGF, VEGF, and HGF. Non-limiting examples of induction agents are cocktails described herein, for example cocktails containing heparin and cAMP. Uniprot Accession Numbers in this paragraph were accessed on May 22, 2017.

It is also provided herein that ASC can be activated by expansion on a 3D substrate, a non-limiting example of which is a carrier comprising a fibrous matrix. The expansion medium may be any of the media described herein, each of which represents a separate embodiment of the present invention.

Basal Medium

Those skilled in the art will appreciate that a variety of isotonic buffers may be used for washing cells and similar uses. Hank's Balanced Salt Solution (MSS; Life Technologies) is only one of many buffers that may be used.

Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 6, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

It will also be appreciated that in certain embodiments, when the described induced cells are intended for administration to a human subject, the cells and the culture medium (e.g., with the above-described medium agents and/or components) are substantially xeno-free, i.e., devoid of any animal components. For example, the culture medium can be supplemented with a serum-replacement, human serum and/or synthetic or recombinantly produced factors.

The various media described herein, i.e. the 2D growth medium and the 3D growth medium, may be independently selected from each of the described embodiments relating to medium composition. In various embodiments, any medium suitable for growth of cells in a bioreactor may be used.

Induction Agents

As a non-limiting example, ASC can be induced by incubation in a medium comprising heparin and cAMP or an analogue thereof. In certain embodiments, a cAMP analogue described herein is a cell-permeable cAMP analog, non-limiting examples of which are dibutyryl cyclic AMP (dbcAMP), 6-Bnz-cAMP (e.g. provided as a sodium salt) (Tocris Bioscience [Bristol, UK)], cat. no. 5255), cAMPS-Sp, (e.g. provided as a triethylammonium salt) (Tocris, cat. no. 1333), and 8-Bromo-cAMP, (e.g. provided as a sodium salt) (Tocris, cat. no. 1140). In some embodiments, the medium is serum-free. In other embodiments, the medium contains serum, which may be, in more specific embodiments, at any of the concentrations mentioned herein.

In more specific embodiments, the concentration of heparin in the medium is 10-200, 10-180, 10-160, 10-140, 10-120, 10-110, 10-100, 15-200, 15-180, 15-160, 15-140, 15-120, 15-110, 15-100, 20-200, 20-180, 20-160, 20-140, 20-120, 20-110, 20-100, 30-200, 30-180, 30-160, 30-140, 30-120, 30-110, 30-100, 40-200, 40-180, 40-160, 40-140, 40-120, 40-110, 40-100, 50-200, 50-180, 50-160, 50-140, 50-120, 50-110, 50-100, 10-50, 15-50, 20-50, 25-50, 30-50, 40-50, 20-80, 25-75, 30-70, 35-75, 40-60, 45-55, 47-53, 48-52, 49-51, about 50, or 50 mcg/ml (micrograms per ml).

In other embodiments, the concentration of the described cAMP or analogue thereof in the medium is 500-2500 mcM (micromolar). In various other embodiments, it is 200-5000, 200-4000, 200-3000, 200-2500, 200-2000, 200-1500, 200-1200, 200-1000, 300-5000, 300-4000, 300-3000, 300-2500, 300-2000, 300-1500, 300-1200, 300-1000, 400-5000, 400-4000, 400-3000, 400-2500, 400-2000, 400-1500, 400-1200, 400-1000, 500-5000, 500-4000, 500-3000, 500-2000, 500-1500, 500-1200, 500-1000, 600-5000, 600-4000, 600-3000, 600-2500, 600-2000, 600-1500, 600-1200, 600-1000, 800-5000, 800-4000, 800-3000, 800-2500, 800-2000, 800-1500, 800-1200, 800-1000, 1000-5000, 1000-4000, 1000-3000, 1000-2500, 1000-2000, 1000-1500, 1000-1200, 250-1000, 300-1000, 350-1000, 400-1000, 500-1000, 600-1000, 700-1000, 800-1000, 900-1000, 500-1500, 500-1800, 600-1400, 600-1800, 700-1300, 700-1400, 700-1500, 800-1200, 900-1100, 950-1050, about 1000, or 1000 mcM.

In still other embodiments, the medium comprises cAMP, or an analogue thereof, for example dbcAMP, at a concentration of 500-2500 mcM and heparin at a concentration of 10-200 mcg/ml. In other embodiments, the respective concentrations of cAMP or an analogue thereof and heparin are 600-2000 mcM and 20-150 mcg/ml; 700-1800 mcM and 25-140 mcg/ml; 800-1600 mcM and 30-120 mcg/ml; 800-1400 mcM and 35-100 mcg/ml; 800-1200 mcM and 35-80 mcg/ml; or 800-1200 mcM and 35-70 mcg/ml.

In certain embodiments, the medium further comprises (in addition to heparin and cAMP or an analogue thereof) one or more induction agents selected from basic fibroblast growth factor (b-FGF; Uniprot Accession No. P09038); PDGF (platelet-derived growth factor; Uniprot Accession Nos. P04085 [subunit A; exemplified herein] and P01127 [subunit B]); and Neuregulin (e.g. Neuregulin 1, non-limiting examples of which are the isoforms HRG-alpha, HRG-beta, HRG-beta2, and HRG-gamma, and the sequences set forth in Uniprot Accession Nos. B7Z168 (or Q7RTV8), Q7RTW4, Q7RTW3, A0A024QY88, Q7RTW5, and B9EK51). (The Uniprot entries in this paragraph were accessed on Nov. 10, 2015). Alternatively or in addition, the medium further comprises a component selected from (a) progesterone; and (b) a polyamine. In other embodiments, the medium comprises both of aforementioned components (a) and (b). In further embodiments, the medium further comprises an additional component selected from (c) transferrin, non-limiting examples of which are apo-transferrin and holo-transferrin; (d) insulin, non-limiting examples of which are full chain insulin and truncated insulin; and (e) selenite. Non-limiting examples of polyamines are putrescine, spermidine, and spermine. In other embodiments, the medium further comprises (in addition to 1 or, in another embodiment, both of components (a)-(b)), 2 or more of aforementioned components (c)-(e). In still other embodiments, the medium further comprises all 3 or more of components (c)-(e). In yet other embodiments, the medium further comprises 2 or more of aforementioned components (a)-(e). In still other embodiments, the medium further comprises 3 or more of components (a)-(e). In yet other embodiments, the medium further comprises 4 or more of components (a)-(e). In further embodiments, the medium further comprises all 5 of components (a)-(e).

Those skilled in the art will appreciate that the precise sequences of b-FGF, PDGF, Neuregulin, and the other induction agents mentioned herein are not typically critical for carrying out the described methods. Alternative isoforms, functional fragments thereof, mimetics thereof, and proteins from non-human species are often suitable, provided that they exhibit biological effects analogous to the active versions.

In more specific embodiments, the concentration of b-FGF in the medium is 5-100 ng/ml (nanograms per milliliter). In various other embodiments, it is 2-100, 3-100, 7-100, 10-100, 15-100, 20-100, 2-80, 3-80, 5-80, 7-80, 10-80, 15-80, 20-80, 2-50, 3-50, 5-50, 7-50, 10-50, 15-50, 20-50, 2-35, 3-35, 5-35, 7-35, 10-35, 15-35, 20-35, 2-20, 3-20, 5-20, 7-20, 10-20, 15-20, 20-80, 20-50, 20-40, 20-30, 10-30, 10-25, 15-30, 15-35, 15-25, 16-24, 17-23, 18-22, 19-21, about 20, or 20 ng/ml.

In still other embodiments, the medium comprises cAMP, or an analogue thereof, for example dbcAMP, at a concentration of 500-2500 mcM; heparin at a concentration of 10-200 mcg/ml; and b-FGF at a concentration of 5-100 ng/ml. In other embodiments, the respective concentrations of cAMP or an analogue thereof, heparin, and b-FGF are 600-2000 mcM, 20-150 mcg/ml, and 6-80 ng/ml; 700-1800 mcM, 25-140 mcg/ml, and 8-60 ng/ml; 800-1600 mcM, 30-120 mcg/ml, and 10-40 ng/ml; 800-1400 mcM, 35-100 mcg/ml, and 12-35 ng/ml; 800-1200 mcM, 35-80 mcg/ml, and 14-30 ng/ml; or 800-1200 mcM, 35-70 mcg/ml, and 16-25 ng/ml.

In more specific embodiments, the concentration of PDGF is 1-20, 1-18, 1-16, 1-14, 1-12, 1-11, 1-10, 1.5-20, 1.5-18, 1.5-16, 1.5-14, 1.5-12, 1.5-11, 1.5-10, 2-20, 2-18, 2-16, 2-14, 2-12, 2-11, 2-10, 3-20, 3-18, 3-16, 3-14, 3-12, 3-11, 3-10, 4-20, 4-18, 4-16, 4-14, 4-12, 4-11, 4-10, 5-20, 5-18, 5-16, 5-14, 5-12, 5-11, 5-10, 1-5, 1.5-5, 2-5, 2.5-5, 3-5, 4-5, 2-8, 2.5-7.5, 3-7, 3.5-7.5, 4-6, 4.5-5.5, 4.7-5.3, 4.8-5.2, 4.9-5.1, about 5, or 5 ng/ml.

In more specific embodiments, the concentration of Neuregulin is 10-200, 10-180, 10-160, 10-140, 10-120, 10-110, 10-100, 15-200, 15-180, 15-160, 15-140, 15-120, 15-110, 15-100, 20-200, 20-180, 20-160, 20-140, 20-120, 20-110, 20-100, 30-200, 30-180, 30-160, 30-140, 30-120, 30-110, 30-100, 40-200, 40-180, 40-160, 40-140, 40-120, 40-110, 40-100, 50-200, 50-180, 50-160, 50-140, 50-120, 50-110, 50-100, 10-50, 15-50, 20-50, 25-50, 30-50, 40-50, 20-80, 25-75, 30-70, 35-75, 40-60, 45-55, 47-53, 48-52, 49-51, about 50, or 50 ng/ml.

In still other embodiments, the medium further comprises (in addition to heparin and cAMP or an analogue thereof) both (i) an induction agent selected from b-FGF, PDGF, and Neuregulin (each of which represents a separate embodiment); and (ii) an additional component selected from: (a) progesterone; and (b) a polyamine. In other embodiments, the medium comprises both of aforementioned components (a) and (b). In yet other embodiments, the medium further comprises an additional component selected from (c) transferrin, non-limiting examples of which are apo-transferrin and holo transferrin; (d) insulin, non-limiting examples of which are full chain insulin and truncated insulin; and (e) selenite. Non-limiting examples of polyamines are putrescine, spermidine, and spermine. In other embodiments, the medium further comprises—in addition to a component selected from b-FGF, PDGF, and Neuregulin; and 1 or, in another embodiment, both of components (a)-(b)-2 or more of aforementioned components (c)-(e). In still other embodiments, the medium further comprises all 3 or more of components (c)-(e). Non-limiting examples of PDGF are PDGF-AA (exemplified herein), PDGF-BB, and PDGF-AB. In some embodiments, the medium is serum-free. In other embodiments, the medium contains serum, which may be, in more specific embodiments, at any of the concentrations mentioned herein.

In yet other embodiments, the medium further comprises both (i) b-FGF, PDGF, or Neuregulin; and (ii) 2 or more of aforementioned components (a)-(e). In yet other embodiments, the medium further comprises both b-FGF, PDGF, or Neuregulin; and 3 or more of components (a)-(e). In yet other embodiments, the medium further comprises both b-FGF, PDGF, or Neuregulin; and 4 or more of components (a)-(e). In yet other embodiments, the medium further comprises both b-FGF, PDGF, or Neuregulin; and all 5 of components (a)-(e).

In certain embodiments, the concentration of additional component (a) is 2-50, 3-50, 4-50, 5-50, 8-50, 10-50, 2-40, 3-40, 4-40, 5-40, 8-40, 10-40, 2-30, 3-30, 4-30, 5-30, 8-30, 10-30, 2-20, 3-20, 4-20, 5-20, 8-20, 10-20, 2-10, 3-10, 4-10, 5-10, 7-10, 8-10, 9-10, 5-15, 6-14, 7-13, 8-12, 9-11, 7-15, 8-20, about 10, or 10 nM (nanomolar).

Alternatively or in addition, the concentration of additional component (b) is 1-20, 1-18, 1-16, 1-14, 1-12, 1-11, 1-10, 1.5-20, 1.5-18, 1.5-16, 1.5-14, 1.5-12, 1.5-11, 1.5-10, 2-20, 2-18, 2-16, 2-14, 2-12, 2-11, 2-10, 3-20, 3-18, 3-16, 3-14, 3-12, 3-11, 3-10, 4-20, 4-18, 4-16, 4-14, 4-12, 4-11, 4-10, 5-20, 5-18, 5-16, 5-14, 5-12, 5-11, 5-10, 1-5, 1.5-5, 2-5, 2.5-5, 3-5, 4-5, 2-8, 2.5-7.5, 3-7, 3.5-7.5, 4-6, 4.5-5.5, 4.7-5.3, 4.8-5.2, 4.9-5.1, about 5, or 5 mg/L (milligrams per liter).

Alternatively or in addition, the concentration of additional component (c) is 1-20, 1-18, 1-16, 1-14, 1-12, 1-11, 1-10, 1.5-20, 1.5-18, 1.5-16, 1.5-14, 1.5-12, 1.5-11, 1.5-10, 2-20, 2-18, 2-16, 2-14, 2-12, 2-11, 2-10, 3-20, 3-18, 3-16, 3-14, 3-12, 3-11, 3-10, 4-20, 4-18, 4-16, 4-14, 4-12, 4-11, 4-10, 5-20, 5-18, 5-16, 5-14, 5-12, 5-11, 5-10, 6-20, 6-18, 6-16, 6-14, 6-12, 6-11, 6-10, 1-6, 1.5-6, 2-6, 2.5-6, 3-6, 4-6, 2-8, 3-7.5, 3-8, 4-8, 4-7, 5-7, 5.5-6.5, about 6, or 6 ng/L.

Alternatively or in addition, the concentration of additional component (d) is 20-500, 20-400, 20-300, 20-250, 20-200, 20-150, 20-120, 20-100, 30-500, 30-400, 30-300, 30-250, 30-200, 30-150, 30-120, 30-100, 40-500, 40-400, 40-300, 40-250, 40-200, 40-150, 40-120, 40-100, 50-500, 50-400, 50-300, 50-250, 50-200, 50-150, 50-120, 50-100, 60-500, 60-400, 60-300, 60-250, 60-200, 60-150, 60-120, 60-100, 80-500, 80-400, 80-300, 80-250, 80-200, 80-150, 80-120, 80-100, 100-500, 100-400, 100-300, 100-250, 100-200, 100-150, 100-120, 25-100, 30-100, 35-100, 40-100, 50-100, 60-100, 70-100, 80-100, 90-100, 50-150, 50-180, 60-140, 60-180, 70-130, 70-140, 70-150, 80-120, 90-110, 95-105, about 100, or 100 nM.

Alternatively or in addition, the concentration of additional component (e) is 100-2000, 100-1800, 100-1600, 100-1400, 100-1200, 100-1100, 100-1000, 1.500-2000, 1.500-1800, 1.500-1600, 1.500-1400, 1.500-1200, 1.500-1100, 1.500-1000, 200-2000, 200-1800, 200-1600, 200-1400, 200-1200, 200-1100, 200-1000, 300-2000, 300-1800, 300-1600, 300-1400, 300-1200, 300-1100, 300-1000, 400-2000, 400-1800, 400-1600, 400-1400, 400-1200, 400-1100, 400-1000, 500-2000, 500-1800, 500-1600, 500-1400, 500-1200, 500-1100, 500-1000, 100-500, 1.500-500, 200-500, 2.500-500, 300-500, 400-500, 200-800, 2.500-7.500, 300-700, 3.500-7.500, 400-600, 4.500-5.500, 4.700-5.300, 4.800-5.200, 4.900-5.100, about 500, or 500 ng/L.

In still other embodiments, N-2 is present in the medium at between 0.2-5, 0.2-4, 0.2-3, 0.2-2, 0.2-1.5, 0.2-1.2, 0.2-1, 0.3-5, 0.3-4, 0.3-3, 0.3-2, 0.3-1.5, 0.3-1.2, 0.3-1, 0.5-5, 0.5-4, 0.5-3, 0.5-2, 0.5-1.5, 0.5-1.2, 0.5-1, 0.6-1.4, 0.6-1.5, 0.7-1.3, 0.7-1.4, 0.8-1.2, 0.8-1.5, 0.8-1.4, 0.9-1.1, about 1, or 1× concentration, where 1× concentration is the usual recommended concentration. N-2 animal-free cell culture supplement is commercially available from ThermoFisher Scientific, Cat. #1752048. 100× N-2 contains 1 mM human transferrin (holo), 500 mg/L (milligrams per liter) Insulin Recombinant Full Chain, 0.63 mg/L progesterone, 10 mM putrescine, and 0.52 mg/L selenite. 1× N-2 contains 10 micromolar (mcM) transferrin, 5 mg/L Insulin, 6.3 mcg/L progesterone, 100 mcM putrescine, and 5.2 mcg/L selenite. In some embodiments, the medium is serum-free. In other embodiments, the medium contains serum, which may be, in more specific embodiments, at any of the concentrations mentioned herein.

In still other embodiments, the medium comprises cAMP, or an analogue thereof, for example dbcAMP, at a concentration of 500-2500 mcM; heparin at a concentration of 10-200 mcg/ml; b-FGF at a concentration of 5-100 ng/ml; and one or more of (a) progesterone at a concentration of 2-20 mcg/L; and (b) a polyamine (e.g. putrescine) at a concentration of 30-300 mcM. In other embodiments, the respective concentrations of cAMP or an analogue thereof, heparin, b-FGF, progesterone, and polyamine are 600-2000 mcM, 20-150 mcg/ml, 6-80 ng/ml, 3-15 mcg/mL and 50-200 mcM; 700-1800 mcM, 25-140 mcg/ml, 8-60 ng/ml, 4-12 mcg/mL and 60-180 mcM; 800-1600 mcM, 30-120 mcg/ml, 10-40 ng/ml, 4-10 mcg/mL and 70-160 mcM; 800-1400 mcM, 35-100 mcg/ml, 12-35 ng/ml, 4-10 mcg/mL and 70-140 mcM; 800-1200 mcM, 35-80 mcg/ml, 14-30 ng/ml, 4-10 mcg/mL and 70-140 mcM; or 800-1200 mcM, 35-70 mcg/ml, 16-25 ng/ml, 5-8 mcg/mL and 80-120 mcM. In certain embodiments, both progesterone and a polyamine are present. In some embodiments, the medium is serum-free. In other embodiments, the medium contains serum, which may be, in more specific embodiments, at any of the concentrations mentioned herein.

In certain embodiments, the induction of ASC is performed on a 2D substrate. In other embodiments, the induction is performed on a 3D substrate. Unless indicated otherwise, a 3D substrate culture apparatus used for induction may be any apparatus mentioned herein, each of which represents a separate embodiment.

In some embodiments, the ASC are expanded ex vivo prior to the step of inducing. For example, the cells may be incubated in a medium lacking one or more induction agents. In more specific embodiments, the ASC are expanded on a 2D substrate, and then induced on a 3D substrate. In some embodiments, the 2D substrate is used for expansion, and the 3D substrate is subsequently used, exclusively for the induction stage. In other embodiments, the 2D substrate is used for expansion, and the 3D substrate is subsequently used for additional cell expansion, followed by cell induction in the 3D substrate.

Induction Methods

In some embodiments, there is provided a method of inducing ASC to secrete a neurotrophic or neuroprotective growth factor, comprising incubating the ASC in a bioreactor, optionally while adhered to a 3D growth substrate and/or in the presence of serum. The medium may be any of the media described herein, each of which represents a separate embodiment.

In other embodiments, there is provided a method of inducing ASC to secrete a neurotrophic or neuroprotective growth factor, comprising incubating the ASC in a medium comprising heparin and cAMP or an analogue thereof. In other embodiments, the medium comprises basic FGF and cAMP or an analogue thereof. In certain embodiments, a cAMP analogue described herein is a cell-permeable cAMP analog, as described herein. In some embodiments, the medium is serum-free. In other embodiments, the medium contains serum, which may be, in more specific embodiments, at any of the concentrations mentioned herein, each of which represents a separate embodiment. In certain embodiments, incubation with heparin and cAMP is performed on a 2D substrate. In other embodiments, the incubation is performed on a 3D substrate. Unless indicated otherwise, a 3D substrate used for induction may be any culture apparatus mentioned herein, each of which represents a separate embodiment.

In other embodiments, the cells are initially expanded in a medium lacking heparin and cAMP, and the medium is exchanged for a medium comprising heparin and cAMP for an additional period of time. In certain embodiments, the cells are incubated in the heparin-and-cAMP-containing induction cocktail for 12-72 hours, in other embodiments, 18-72 hours, 18-60 hours, 18-48 hours, 18-36 hours, 20-36 hours, 20-30 hours, or 20-28 hours. The heparin-containing induction cocktail may be any cocktail described herein, each of which represents a separate embodiment. Incubation in the heparin-containing cocktail may be, in various embodiments, on a 2D- or 3D-substrate.

In certain embodiments, ASC are induced by the described methods to increase over baseline levels induction of neurotrophic and/or neuroprotective growth factors by the described methods.

In other embodiments, the step of incubating ASC in a bioreactor is preceded by incubation in serum-free medium (SFM), or, in more specific embodiments, serum replacement medium (SRM; defined herein). In yet other embodiments, the step of incubating ASC with heparin and cAMP is preceded by incubation in SRM, on in other embodiments SFM. In certain embodiments, the incubation in SRM or SFM begins from the stage of extraction from the placenta. In other embodiments, serum-containing medium is initially used, and then culturing in SRM or SFM is commenced within 5 days after extraction, or in other embodiments 1 passage after extraction, or in other embodiments prior to the first passage after extraction. In certain embodiments, the initial serum-containing medium does not comprise added heparin or cAMP. In certain embodiments, the incubation in SRM or SFM continues in a tissue culture apparatus for at least 3 passages, at least 4 passages, at least 5 passages, or at least 6 passages.

As mentioned, in some embodiments, an induction medium (comprising an induction cocktail) is added following the incubation in SRM, and the cells are incubated for an additional period of time. In certain embodiments, the induction cocktail contains heparin and/or cAMP or an analogue thereof. Alternatively or in addition, the cells are incubated in the induction cocktail for 12-72 hours, in other embodiments, 18-72 hours, 18-60 hours, 18-48 hours, 18-36 hours, 20-36 hours, 20-30 hours, or 20-28 hours. The induction cocktail may be any cocktail described herein each of which represents a separate embodiment. Incubation in the cocktail may be, in various embodiments, on a 2D- or 3D-substrate. In certain embodiments, ASC are incubated in a serum-containing medium between the SRM and the induction medium.

In other embodiments, serum-containing medium is used for the initial 2-5 population doublings, on in other embodiments 2-20, 2-15, 2-10, 2-8, or 2-6 population doublings after the first passage. Those skilled in the art will appreciate that it may be difficult to determine an exact population doubling level (PDL) between extraction of cells from tissue and the first passage. In such case, if necessary the population doublings at this first stage may be estimated. Typical population doubling values prior to the first passage are below 5, often ranging from 2-5. In certain embodiments, the initial serum-containing medium does not comprise added heparin or cAMP.

In certain embodiments, the described step of incubating the ASC population in serum-free medium, or in other embodiments in SRM, is performed for at least 12, at least 15, at least 17, at least 18, 12-30, 12-25, 15-30, 15-25, 16-25, 17-25, or 18-25 doublings.

In other embodiments, the ASC population is incubated in SFM, or in other embodiments in SRM, for a defined number of passages, for example 1-4, 1-3, 1-2, 2-4, or 2-3; or a defined number of population doublings, for example at least 4, at least 5, at least 6, at least 7, at least 8, 4-10, 4-9, 4-8, 5-10, 5-9, or 5-8. The cells are then cryopreserved, then subjected to additional culturing in SRM or SFM, prior to induction. In some embodiments, the additional culturing in SRM or SFM is performed for at least 6, at least 7, at least 8, at least 9, at least 10, 6-20, 7-20, 8-20, 9-20, 10-20, 6-15, 7-15, 8-15, 9-15, or 10-15 population doublings. Alternatively, the additional culturing in SRM is performed for 2-3 passages, or in other embodiments at least 1, at least 2, at least 3, 1-5, 1-4, 1-3, 2-5, or 2-4 passages.

Each of the described embodiments of culturing ASC in SFM or in SRM may be followed by incubation in a bioreactor, in in other embodiments, incubation with heparin and cAMP.

In some embodiments, ASC are incubated in SRM (or in other embodiments SFM), followed by serum-containing medium, prior to induction by incubation in a bioreactor, in in other embodiments, by incubation with heparin and cAMP. In other embodiments, serum-containing medium is initially used, then ASC are incubated in SRM (or SFM), then once again in serum-containing medium, prior to induction by incubation in a bioreactor, in in other embodiments, incubation with heparin and cAMP. In other embodiments, the induction is performed in serum-containing medium comprising heparin and cAMP.

In certain embodiments, the ASC are expanded in SRM (or SFM) on a 2D substrate, followed by induction on a 3D substrate. In other embodiments, the 2D substrate is used for expansion, and the 3D substrate is subsequently used for additional cell expansion, followed by induction in the 3D substrate. In other embodiments, SRM (or SFM) is utilized for part of incubation on a 2D matrix, after which serum-containing medium is utilized for the remainder of incubation on a 2D matrix, and also for incubation on a 3D matrix—which occurs, in some embodiments, in a bioreactor—after which the ASC are induced with heparin and cAMP. In other embodiments, the incubation in SRM (or SFM) continues until seeding of the cells in serum-containing medium on a 3D matrix—which occurs, in some embodiments, in a bioreactor—after which the ASC are then induced with heparin and cAMP. In other embodiments, the induction is performed in serum-containing medium comprising heparin and cAMP.

The described serum-containing medium, in certain embodiments, contains 5-30% serum (non-limiting examples of which are fetal bovine serum and fetal calf serum). In more specific embodiments, the medium contains over 10% serum; 10-30% serum; 12-28% serum; 14-26% serum; 16-24% serum; 17-23% serum; 18-22% serum; 19-21% serum; about 20% serum; or 20% serum. The serum-containing medium used at the described stages may be varied independently, and each possibility represents a separate embodiment.

It is clarified that the embodiments wherein the serum-containing medium comprises one or more of the aforementioned induction agents (non-limiting examples of which are bFGF, dbcAMP, heparin, and N-2 supplement) are not excluded from the present disclosure. In still other embodiments, the serum-containing medium further comprises contains N-2 or at least 2 components thereof (for example progesterone and a polyamine (e.g. putrescine). In more specific embodiments, the serum-containing medium further comprises at least 3, at least 4, or all 5 N-2 components.

Alternatively, incubation in SRM (as described) is followed by incubation in a a subsequent medium which is serum-free and does not contain any of the following: heparin, cAMP or an analogue thereof, b-FGF, PDGF, or Neuregulin. In certain embodiments, the subsequent medium comprises added cytokines, not including any of heparin, cAMP or an analogue thereof, b-FGF, PDGF, or Neuregulin. Incubation in SRM is, in some embodiments, preceded by incubation in serum-containing medium. In any case, incubation in the described subsequent medium is followed by induction by incubation in a bioreactor, or in other embodiments, by incubation with heparin and cAMP, as described herein.

Serum-Free (SF) and SRM Media

As mentioned, in certain embodiments, the described methods utilize SF medium. In some embodiments, the SF medium is supplemented with factors intended to stimulate cell expansion in the absence of serum. Such medium is referred to herein as serum-replacement medium or SRM, and its use, for example in cell culture and expansion, is well known in the art, and is described, for example, in Kinzebach et al.

It is clarified that the described factors intended to stimulate cell expansion in the absence of serum may also be present (together with the induction agents) in the described induction medium.

SRM formulations include MSC Nutristem® XF (Biological Industries); Stempro® SFM and Stempro® SFM-XF (Thermo Fisher Scientific); PPRF-msc6; D-hESF10; TheraPEAK™ MSCGM-CDTM (Lonza, cat. no. 190632); and MesenCult-XF (Stem Cell Technologies, cat. no. 5429). The StemPro® media contain PDGF-BB, bFGF, and TGF-β, and insulin. The composition of PPRF-msc6 is described in US 2010/0015710, which is incorporated herein by reference. D-hESF10 contains insulin (10 mcg/ml); transferrin (5 mcg/ml); oleic acid conjugated with bovine albumin (9.4 mcg/ml); FGF-2 (10 ng/ml); and TGF-β1 (5 ng/ml), as well as heparin (1 mg/ml) and standard medium components (Mimura et al).

As provided herein, ASC were expanded in Stempro® SFM-XF, prior to incubation in serum, and, subsequently, induction. MSC Nutristem® XF was also tested and yielded similar results. Additionally, medium containing PDGF-BB, bFGF, and TGF-β, added to DMEM/F-12, was tested and yielded similar results. DMEM/F-12 is a known basal medium, available commercially from Thermo Fisher Scientific (cat. no. 10565018).

Another SRM formulation is described in Rajaraman G et al and contains FGF-2 (10 ng/ml); epidermal growth factor (EGF) (10 ng/ml); 0.5% BSA; Insulin (10 mcg/ml); transferrin (5.5 mcg/ml); 6.7 ng/mL sodium selenite, sodium pyruvate (11 mcg/ml); heparin (0.1 mg/ml); 10 nM linolenic acid.

Another SRM formulation for human stromal cells is described in U.S. Pat. No. 5,908,782 to Marshak and Holecek, incorporated herein by reference.

Other commercially available media include BD Mosaic™ hMSC serum-free medium (cat. no. 355701, BD Biosciences), CellGro™ (cat. no. 24803-0500, CellGenix, containing insulin, albumin, and lecithin), HEScGRO (cat. no. SCM020, Merck Millipore), Mesenchymal stem cell growth medium DXF (cat. no. C-28019, PromoCell), MesenGro (cat. no. ZRD-MGro-500, StemRD), MSC Qualified PLUS™ (cat. no. PLS2, Compass Biomedical), MSC-Gro™ (SF, complete) (cat. no. SCO0B3, Vitro Biopharma), MSCGS-ACF (cat. no. 7572, ScienCell Research, mTeSR (cat. no. 5850, Stem Cell Technologies), PRIME-XVTM MSC Expansion SFM (cat. no. 31000, Irvine Scientific), RS-Novo™ and GEM-Novo (Kerry Bio-Sciences), MSCM-sf (ScienCell™), SPE-IV (cat. no. SPE-IV-EBM/500, Abecell-Bio), Stemline MSC expansion medium (cat. no. S1569, Sigma Aldrich), StemXVivo™ (cat. no. CCM014, R&D Systems, Inc), STK2 (Two Cells Co., Ltd.), and Ultrasor G (lyophilized) (cat. no. 15950-017, Pall Biosepra).

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF (platelet-derived growth factor, a non-limiting example of which is PDGF-BB). In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

In other embodiments, the described SRM comprises bFGF, TGF-β, and insulin. In additional embodiments, a component selected from transferrin (5 mcg/ml) and oleic acid are present; or both transferrin and oleic acid are present. Oleic acid can be, in some embodiments, conjugated with a protein, a non-limiting example of which is albumin. In some embodiments, the SRM comprises 5-20 ng/ml bFGF, 2-10 ng/ml TGF-β, and 5-20 ng/ml insulin, or, in other embodiments, 7-15 ng/ml bFGF, 3-8 ng/ml TGF-β, and 7-15 ng/ml insulin. In more specific embodiments, a component selected from 2-10 mcg/ml transferrin and 5-20 mcg/ml oleic acid, or in other embodiments, a component selected from 3-8 mcg/ml transferrin and 6-15 mcg/ml oleic acid, or in other embodiments the aforementioned amounts of both components (transferrin and oleic acid) is/are also present.

In still other embodiments, the SRM further comprises a component, or in other embodiments 2, 3, or 4 components, selected from ethanolamine, glutathione, ascorbic acid, and albumin. Alternatively or in addition, the SRM further comprises a trace element, or in other embodiments, 2, 3, 4, or more than 4 trace elements. In some embodiments, the trace element(s) are selected from selenite, vanadium, copper, and manganese.

In still other embodiments, the described SRM comprises platelet lysate (van den Dolder et al).

In other embodiments, the described SRM comprises bFGF and epidermal growth factor (EGF). In more specific embodiments, the bFGF and EGF are present at concentrations independently selected from 5-40, 5-30, 5-25, 6-40, 6-30, 6-25, 7-40, 7-30, 7-25, 7-20, 8-, 8-17, 8-15, 8-13, 9-20, 9-17, 9-15, 10-15, 5-20, 5-10, 7-13, 8-12, 9-11, or 10 ng/ml. In certain embodiments, insulin; and/or transferrin is also present. In more specific embodiments, the insulin and transferrin are present at respective concentrations of 5-20 and 2-10; 6-18 and 3-8; or 8-15 and 4-7 mcg/ml. Alternatively or in addition, the SRM further comprises an additional component selected from BSA, selenite (e.g. sodium selenite), pyruvate (e.g. sodium pyruvate); heparin, and linolenic acid. In other embodiments 2 or more, or in other embodiments 3 or more, in other embodiments 4 or more, or in other embodiments all 5 of BSA, selenite, pyruvate, heparin, and linolenic acid are present. In more specific embodiments, the BSA, selenite, pyruvate, heparin, and linolenic acid are present at respective concentrations of 0.1-5%, 2-30 ng/mL, 5-25 mcg/ml, 0.05-0.2 mg/ml, and 5-20 nM; or in other embodiments at respective concentrations of 0.2-2%, 4-10 ng/mL, 7-17 mcg/ml, 0.07-0.15 mg/ml, and 7-15 nM; or in other embodiments the aforementioned amounts or 2 or more, or in other embodiments 3 or more, in other embodiments 4 or more, or in other embodiments all 5 of BSA, selenite, pyruvate, heparin, and linolenic acid are present.

In other embodiments, bFGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 8-13, 8-12, 9-11, 9-12, about 10, or 10 nanograms per milliliter (ng/ml).

In other embodiments, EGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/ml.

In other embodiments, TGF-β, where present, is present at a concentration of 1-25, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 ng/ml.

In other embodiments, PDGF, where present, is present at a concentration of 1-50, 1-40, 1-30, 1-20, 1-15, 1-10, 1-8, 1-7, 1-6, 1-5, 1-4, 1-3, 1-2, 2-50, 2-40, 2-30, 2-20, 2-15, 2-10, 2-8, 2-7, 2-6, 2-5, 2-4, 3-50, 3-40, 3-30, 3-20, 3-15, 3-10, 3-8, 3-7, 3-6, 3-5, 3-4, 4-40, 4-30, 4-20, 5-40, 5-30, 5-20, 5-15, 5-12, 5-10, 10-20, 10-18, 10-16, or 10-15, 2-20, about 2, about 3, about 5, about 10, about 15, about 20, 2, 3, 5, 10, 15, or 20 ng/mL.

In other embodiments, insulin, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 micrograms per milliliter (mcg/ml).

In other embodiments, transferrin, where present, is present at a concentration of 1-25, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 mcg/ml.

In other embodiments, heparin, where present, is present at a concentration of 10-400, 10-300, 10-200, 20-400, 20-300, 20-200, 30-400, 30-300, 30-200, 30-150, 40-300, 40-200, 40-150, 50-300, 50-200, 50-150, 60-140, 70-140, 70-250, 70-220, 80-250, 80-220, 90-210, 100-200, 80-130, 80-120, 90-110, 90-120, about 100, or 100 ng/ml.

In other embodiments, linolenic acid, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 nanomolar (nM).

In other embodiments, stem cell factor (SCF) is present in the SRM. In certain embodiments, SCF is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/mL.

In other embodiments, insulin-like growth factor-1 and/or 2 (IGF-1 and/or IGF-2) is present in the SRM. In more specific embodiments, IGF-1 and/or IGF-2 is present at a concentration of 10-250, 20-250, 30-250, 40-250, 50-250, 10-200, 10-150, 10-100, 10-80, 10-70, 10-60, 10-50, 20-200, 20-150, 20-100, 30-200, 30-150, 30-100, 30-80, 30-70, 40-80, 40-70, 40-60, 45-55, about 50, or 50 ng/mL.

In other embodiments, Keratinocyte Growth Factor (KGF) is present in the SRM. In more specific embodiments, KGF is present at a concentration of 5-100, 5-80, 5-60, 5-50, 5-40, 5-30, 5-20, 10-100, 10-80, 10-60, 10-50, 10-40, 10-30, 10-20, 20-100, 20-80, 20-60, 20-50, 20-40, 20-30, 15-25, 17-23, 18-22, 19-22, about 20, or 20 ng/mL.

In other embodiments, Interleukin 3 (IL-3) is present in the SRM. In more specific embodiments, IL-3 is present at a concentration of 0.5-10, 0.5-8, 0.5-6, 0.5-5, 0.5-4, 0.5-3, 0.5-2, 1-10, 1-8, 1-6, 1-5, 1-4, 1-3, 1-2, 2-10, 2-8, 2-6, 2-5, 2-4, 2-3, 1.5-2.5, 1.7-2.3, 1.8-2.2, 1.9-2.2, about 2, or 2 ng/mL.

In other embodiments, Interleukin 7 (IL-7) is present in the SRM. In more specific embodiments, IL-7 is present at a concentration of 0.5-10, 0.5-8, 0.5-6, 0.5-5, 0.5-4, 0.5-3, 0.5-2, 1-10, 1-8, 1-6, 1-5, 1-4, 1-3, 1-2, 2-10, 2-8, 2-6, 2-5, 2-4, 2-3, 1.5-2.5, 1.7-2.3, 1.8-2.2, 1.9-2.2, about 2, or 2 ng/mL.

2D and 3D Culturing Embodiments

In certain embodiments, the described ASC are subject to a 3D incubation, as described further herein. In more specific embodiments, the ASC have been incubated in a 2D adherent-cell culture apparatus, prior to the step of 3D culturing. In some embodiments, cells (which have been extracted, in some embodiments, from placenta, from adipose tissue, etc.) are then subjected to prior step of incubation in a 2D adherent-cell culture apparatus, followed by the described 3D culturing steps.

In certain embodiments, induction is performed in a 3D culturing apparatus. Each embodiment of induction and 3D culturing may be freely combined in this regard. In certain embodiments, the induced cells are then removed from the 3D culturing apparatus into a pharmaceutical composition.

In other embodiments, induction is performed in a 2D culturing apparatus. Each embodiment of induction and 2D culturing may be freely combined in this regard. In certain embodiments, the induced cells are then removed from the 2D culturing apparatus into a pharmaceutical composition.

The phrase “two-dimensional culture” refers to a culture in which the cells are exposed to conditions that are compatible with cell growth and allow the cells to grow in a monolayer, which is referred to as a “two-dimensional culture apparatus”. Such apparatuses will typically have flat growth surfaces (also referred to herein as a “2D substrate”), in some embodiments comprising an adherent material, which may be planar or curved. Non-limiting examples of apparatuses for 2D substrate culture are cell culture dishes and plates. Included in this definition are multi-layer trays, such as Cell Factory™ manufactured by Nunc™, provided that each layer supports monolayer culture. It will be appreciated that even in 2D substrate apparatuses, cells can grow over one another when allowed to become over-confluent. This does not affect the classification of the apparatus as “two-dimensional”.

The terms “three-dimensional culture” and “3D culture” (either in the context of cell expansion, or, in other embodiments, in the context of induction) refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to herein as a “3D substrate”), in some embodiments comprising an adherent material, which is present in the 3D substrate culture apparatus, e.g. a bioreactor. Certain, non-limiting embodiments of 3D culturing conditions suitable for expansion of adherent stromal cells are described in PCT Application Publ. No. WO/2007/108003, which is fully incorporated herein by reference in its entirety.

In certain embodiments, 3D culturing can be performed in a 3D bioreactor. In some embodiments, the 3D bioreactor comprises a container for holding medium and a 3-dimensional attachment substrate disposed therein, and a control apparatus, for controlling pH, temperature, and oxygen levels and optionally other parameters. The terms attachment substrate and growth substrate are interchangeable. In certain embodiments, the attachment substrate is in the form of carriers, which comprise, in more specific embodiments, a surface comprising a synthetic adherent material. Alternatively or in addition, the bioreactor contains ports for the inflow and outflow of fresh medium and gases. Unless indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

As provided herein, a 3D bioreactor is capable, in certain embodiments, of 3D expansion of adherent stromal cells under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are packed-bed bioreactors. The term packed-bed bioreactor, except where indicated otherwise, refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during the standard perfusion rate of the bioreactor. In certain embodiments, the definition does not exclude that the substrate may be lifted at unusually fast perfusion rates, for example greater than 200 rpm.

In other embodiments, an air-lift bioreactor is used, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference. For example, the substrate may have sufficient density to prevent being lifted and/or it may be packed by mechanical pressure to present it from being lifted. The substrate may be either a single body or multiple bodies. Typically, the substrate remains substantially in place during the standard perfusion rate of the bioreactor. In certain embodiments, the substrate may be lifted at unusually fast perfusion rates, for example greater than 200 rpm.

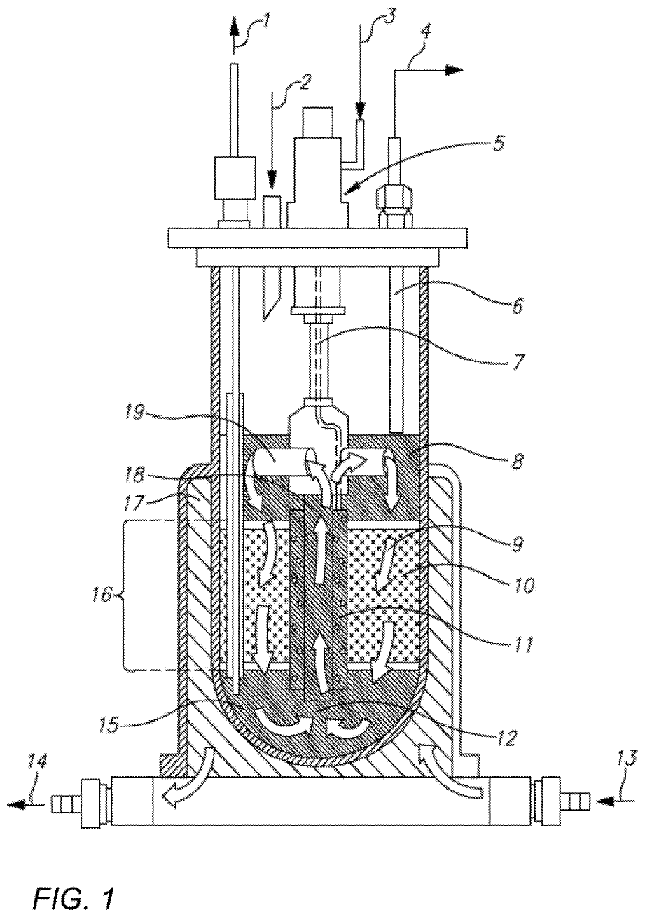

Another exemplary bioreactor, the Celligen 310 Bioreactor, is depicted in FIG. 1. In the depicted embodiment, a Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow initial stirring rate is used to promote cell attachment, then the stirring rate is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

Alternatively or in addition, the adherent material is fibrous, which may be, in more specific embodiments, a woven fibrous matrix, a non-woven fibrous matrix, or either. In still other embodiments, the material exhibits a chemical structure such as charged surface groups, which allows cell adhesion, e.g. polyesters, polypropylenes, polyalkylenes, polyfluorochloroethylenes, polyvinyl chlorides, polystyrenes, polysulfones, cellulose acetates, and poly-L-lactic acids. In more particular embodiments, the material may be selected from a polyester and a polypropylene.

Alternatively or in addition, the carriers comprise a fibrous material, optionally an adherent, fibrous material, which may be, in more specific embodiments, a woven fibrous matrix, a non-woven fibrous matrix, or either. Non-limiting examples of fibrous carriers are New Brunswick Scientific Fibracel® carriers, available commercially from of Eppendorf AG, Germany, and made of polyester and polypropylene; and BioNOC II carriers, available commercially from CESCO BioProducts (Atlanta, Ga.) and made of PET (polyethylene terephthalate). In certain embodiments, the referred-to fibrous matrix comprises a polyester, a polypropylene, a polyalkylene, a polyfluorochloroethylene, a polyvinyl chloride, a polystyrene, or a polysulfone. In more particular embodiments, the fibrous matrix is selected from a polyester and a polypropylene.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; a non-woven fibrous matrix; non-woven fibrous matrix comprising polyester; or a non-woven fibrous matrix comprising at least about 50% polyester. In more specific embodiments, the matrix may be similar to the Celligen™ Plug Flow bioreactor which is, in certain embodiments, packed with Fibra-cel® (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the stirring speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, carriers are removed from the packed bed, washed with isotonic buffer, and processed or removed from the carriers by agitation and/or enzymatic digestion.

The length of the described 3D culturing, in other embodiments, is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings. In some embodiments, the described lengths describes the total time in a 3D substrate culture apparatus, including the expansion and induction stages.

In other embodiments, the cells are induced on a 3D substrate for 2-6 days; in other embodiments, 2-5 days; in other embodiments, 2-4 days; in other embodiments, 2-3 days; in other embodiments, 1-6 days; in other embodiments, 1-5 days; in other embodiments, 1-4 days; in other embodiments, 1-3 days; in other embodiments, 3-6 days; in other embodiments, 3-5 days; in other embodiments, 3-4 days. In other embodiments, the cells are induced on a 3D substrate for 2-6 doublings; in other embodiments, 2-5 doublings; in other embodiments, 2-4 doublings; in other embodiments, 2-3 doublings; in other embodiments, 1-6 doublings; in other embodiments, 1-5 doublings; in other embodiments, 1-4 doublings; in other embodiments, 1-3 doublings; in other embodiments, 3-6 doublings; in other embodiments, 3-5 doublings; in other embodiments, 3-4 doublings. Each of these lengths of 3D induction may be freely combined with each of the aforementioned lengths of total 3D substrate culture, in cases where the length of total 3D substrate culture is at least as long at the length of 3D induction.

In still other embodiments, the cells are induced on a 2D substrate for 2-6 days; in other embodiments, 2-5 days; in other embodiments, 2-4 days; in other embodiments, 2-3 days; in other embodiments, 1-6 days; in other embodiments, 1-5 days; in other embodiments, 1-4 days; in other embodiments, 1-3 days; in other embodiments, 3-6 days; in other embodiments, 3-5 days; in other embodiments, 3-4 days. In other embodiments, the cells are induced on a 2D substrate for 2-6 doublings; in other embodiments, 2-5 doublings; in other embodiments, 2-4 doublings; in other embodiments, 2-3 doublings; in other embodiments, 1-6 doublings; in other embodiments, 1-5 doublings; in other embodiments, 1-4 doublings; in other embodiments, 1-3 doublings; in other embodiments, 3-6 doublings; in other embodiments, 3-5 doublings; in other embodiments, 3-4 doublings.

In certain embodiments, the described bioreactor is seeded at a concentration of between 10,000-2,000,000 cells/ml of medium, in other embodiments 20,000-2,000,000 cells/ml, in other embodiments 30,000-1,500,000 cells/ml, in other embodiments 40,000-1,400,000 cells/ml, in other embodiments 50,000-1,300,000 cells/ml, in other embodiments 60,000-1,200,000 cells/ml, in other embodiments 70,000-1,100,000 cells/ml, in other embodiments 80,000-1,000,000 cells/ml, in other embodiments 80,000-900,000 cells/ml, in other embodiments 80,000-800,000 cells/ml, in other embodiments 80,000-700,000 cells/ml, in other embodiments 80,000-600,000 cells/ml, in other embodiments 80,000-500,000 cells/ml, in other embodiments 80,000-400,000 cells/ml, in other embodiments 90,000-300,000 cells/ml, in other embodiments 90,000-250,000 cells/ml, in other embodiments 90,000-200,000 cells/ml, in other embodiments 100,000-200,000 cells/ml, in other embodiments 110,000-1,900,000 cells/ml, in other embodiments 120,000-1,800,000 cells/ml, in other embodiments 130,000-1,700,000 cells/ml, in other embodiments 140,000-1,600,000 cells/ml. In other embodiments, the cell concentration at time of induction is within any of the aforementioned ranges.

In still other embodiments, between 1-20×10⁶ cells per gram (gr) of carrier (substrate) are seeded, or in other embodiments 1.5-20×10⁶ cells/gr carrier, or in other embodiments 1.5-18×10⁶ cells/gr carrier, or in other embodiments 1.8-18×10⁶ cells/gr carrier, or in other embodiments 2-18×10⁶ cells/gr carrier, or in other embodiments 3-18×10⁶ cells/gr carrier, or in other embodiments 2.5-15×10⁶ cells/gr carrier, or in other embodiments 3-15×10⁶ cells/gr carrier, or in other embodiments 3-14×10⁶ cells/gr carrier, or in other embodiments 3-12×10⁶ cells/gr carrier, or in other embodiments 3.5-12×10⁶ cells/gr carrier, or in other embodiments 3-10×10⁶ cells/gr carrier, or in other embodiments 3-9×10⁶ cells/gr carrier, or in other embodiments 4-9×10⁶ cells/gr carrier, or in other embodiments 4-8×10⁶ cells/gr carrier, or in other embodiments 4-7×10⁶ cells/gr carrier, or in other embodiments 4.5-6.5×10⁶ cells/gr carrier.

In certain embodiments, the harvest from the bioreactor is performed when at least about 10%, in other embodiments at least 12%, in other embodiments at least 14%, in other embodiments at least 16%, in other embodiments at least 18%, in other embodiments at least 20%, in other embodiments at least 22%, in other embodiments at least 24%, in other embodiments at least 26%, in other embodiments at least 28%, or in other embodiments at least 30%, of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In other embodiments, the described incubation of ASC comprises microcarriers, which may, in certain embodiments, be inside a bioreactor. Microcarriers are well known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.) Superbeads (commercially available from Flow Labs, Inc.), and as DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

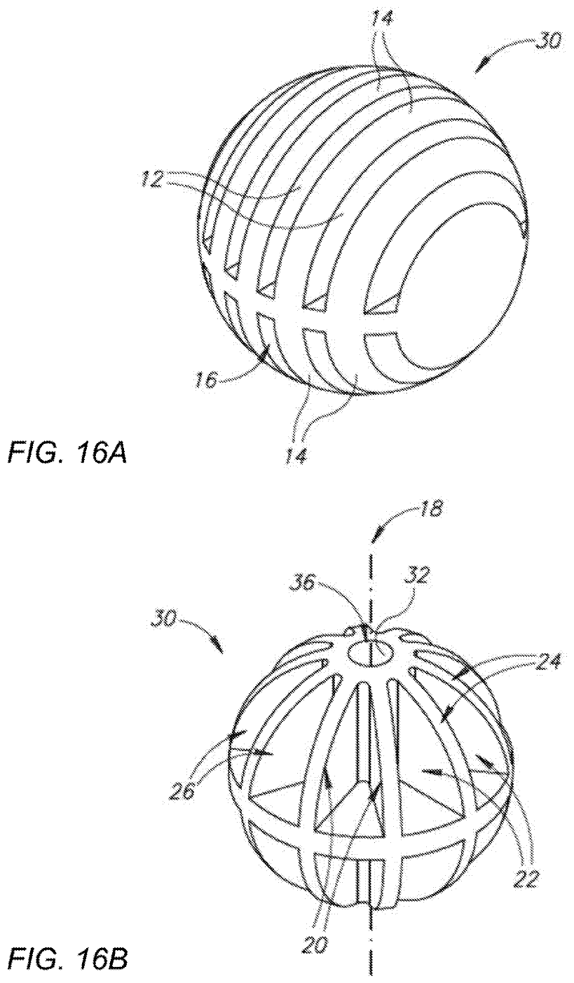

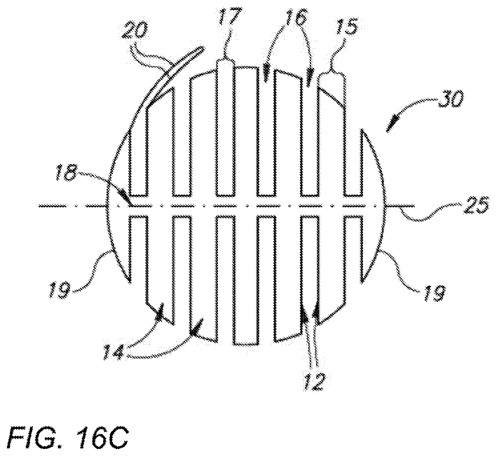

In some embodiments, with reference to FIGS. 16A-B, and as described in WO/2014/037862, published on Mar. 13, 2014, which is incorporated herein by reference in its entirety, grooved carriers 30 are used for proliferation and/or incubation of ASC. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate. Carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 16C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In some embodiments, carriers 30 are “3D bodies” as described in WO/2014/037862; the contents of which relating to 3D bodies are incorporated herein by reference.

In certain embodiments, the described carriers (e.g. grooved carriers) are used in a bioreactor. In some, the carriers are in a packed conformation.

In still other embodiments, the material forming the multiple 2D surfaces comprises at least one polymer. Suitable coatings may, in some embodiments, be selected to control cell attachment or parameters of cell biology.

Agitation Harvesting

In certain embodiments, the described method further comprises the subsequent step (following the described 3D expansion, or, in other embodiments, following the described induction in a 3D apparatus) of harvesting the induced cells by removing the induced cells from the 3D substrate. In more specific embodiments, the harvesting process comprises agitation. In certain embodiments, the agitation utilizes vibration, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are well known in the art; non-limiting examples are TrypLE™, a fungal trypsin-like protease, and Collagenase, Types I, II, III, and IV, which are available commercially from Life Technologies. Enzymes with similar activity to collagenase are well known in the art; non-limiting examples are Dispase I and Dispase II, which are available commercially from Sigma-Aldrich. In still other embodiments, the cells are harvested by a process comprising an optional wash step, followed by incubation with collagenase, followed by incubation with trypsin. In various embodiments, at least one, at least two, or all three of the aforementioned steps comprise agitation. In more specific embodiments, the total duration of agitation during and/or after treatment with protease plus a calcium chelator is between 2-10 minutes, in other embodiments between 3-9 minutes, in other embodiments between 3-8 minutes, and in still other embodiments between 3-7 minutes. In still other embodiments, the cells are subjected to agitation at 0.7-6 Hertz, or in other embodiments 1-3 Hertz, during the wash step before the protease and calcium chelator are added. Alternatively or in addition, the ASC are expanded using an adherent material in a container, which is in turn disposed within a bioreactor chamber; and an apparatus is used to impart a reciprocating motion to the container relative to the bioreactor chamber, wherein the apparatus is configured to move the container in a manner causing cells attached to the adherent material to detach from the adherent material. In more specific embodiments, the vibrator comprises one or more controls for adjusting amplitude and frequency of the reciprocating motion. Alternatively or in addition, the adherent material is a 3D substrate, which comprises, in some embodiments, carriers comprising a synthetic adherent material.

Alternatively or in addition, the cells are cryopreserved following any of the aforementioned induction and/or harvesting steps.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Intermediate Cell Stock Production

Methods

The procedure included periodic testing of the medium for sterility and contamination.

### Step 1-1—Extraction and Plating of Adherent Stromal Cells (ASC's)

Placentas were obtained from donors up to 35 years old, who were pre-screened and determined to be negative for hepatitis B, hepatitis C, HIV-1 and HIV-2, HTLV-1 and HTLV-2, and syphilis. The donor placenta was maintained sterile and cooled.

Within 36 hours of the delivery, the placenta (apart from the amnion and chorion) was placed with the maternal side facing upwards and minced. Pieces were washed with isotonic buffer+gentamicin, then incubated for 1-3 hours with collagenase and DNAse in isotonic buffer. DMEM with 10% filtered FBS, L-Glutamine, and gentamicin was added, and cells were filtered through a sterile stainless steel sieve and centrifuged. The cells were suspended in culture medium, seeded in flasks, and incubated at 37° C. in a humidified tissue culture incubator with 5% CO₂.

After 2 days, cells were washed with PBS, and CellStart™ cell attachment solution and StemPro® MSC SFM XenoFree medium (serum-free and xeno-free culture medium [SFM-XF]) (ThermoFisher Scientific, catalog no. A10675-01; hereinafter “StemPro® medium”) were added.

### Step 1-2—Initial Culturing

Cells were cultured for 2 additional passages (typically 4-10 population doublings after the first passage) in StemPro® medium+ CellStart™. When reaching 60-90% confluence, cells were detached using trypsin, centrifuged, and seeded at 3.16±0.5×10³ cells/cm² in tissue culture flasks.

### Step 1-3—Cell Concentration, Washing, Formulation, Filling and Cryopreservation

The cell suspension from the final passage was centrifuged and suspended in culture medium at 20-40×10⁶ cells/milliliter (mL), then adjusted to 10% DMSO, 40% FBS, and 50% DMEM, the temperature was reduced in a controlled rate freezer, and cells were stored in a liquid nitrogen freezer to produce the ICS.

Results

Cell characteristics of several batches were assessed (Table 2).

TABLE 2
Characteristics of placental cells expanded in SF medium.

| | | | Total growth | cell size | |
|---|---|---|---|---|---|
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 20 | 18.9 | 6.9 |
| PD240214SFM | A | 1 | 7 | 16.2 | NA |
| | | 2 | 14 | 20.8 | 2.7 |
| | | 3 | 20 | 19.4 | 6.4 |
| | B | 1 | 7 | 22 | NA |
| | | 2 | 14 | 18.2 | 2.1 |
| | | 3 | 20 | 19.2 | 6.1 |
| PD230414SFM | NA | 1 | 7 | NA | NA |
| | | 2 | 14 | NA | 2.3 |
| | | 3 | 19 | 16.2 | 5.7 |
| PD040514SFM | NA | 1 | 7 | NA | NA |
| | | 2 | 14 | NA | 2.7 |
| | | 3 | 18 | 15.6 | 6.5 |
| PD260514SFM | NA | 1 | 7 | NA | NA |
| | | 2 | 13 | NA | 2.9 |
| | | 3 | 17 | 15.8 | 6.6 |
| PD180814SFM | NA | 1 | 6 | NA | NA |
| | | 2 | 10 | NA | 2.1 |
| | | 3 | 16 | 16.7 | 5.3 |
| PD220914SFM | unfiltered | 1 | 8 | NA | NA |
| | | 2 | 14 | NA | 2.1 |
| | | 3 | 20 | 17 | 5.6 |
| | filtered | 1 | 8 | NA | NA |
| | | 2 | 14 | NA | 2 |
| | | 3 | 20 | 17.8 | 5.1 |
| PD271014SFM | filtered | 1 | 9 | NA | NA |
| | | 2 | 15 | NA | 2.1 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

PDL refers to population doubling level-in this case, the number of doublings since passage 1.

### Example 2: Further Culture and Induction of ASC

Methods

### Step 2-1: Further Serum-Free Culturing

The ICS was thawed, washed with and cultured in StemPro® medium until 60⁻90% confluence (typically 4-7 days after seeding), and cultured for 2 additional passages (passages “3/1” and “3/2”)), then were harvested for an additional cryopreservation.

Step 2-2: Further Culturing with Serum

2D cell growth in serum-containing medium for one passage (“3/3”). In some cases, cells were switched to serum-containing medium for the final 3 days of passage 3/2.

Step 2-3: Induction with Induction Agents

After passage 3/3, 2×10\hat{ }5 placenta-derived cells were diluted in DMEM+10% FBS, centrifuged, and suspended and seeded in 6-well plates, in 2 ml DMEM+10% FBS medium per well. After 1 day, the medium was aspirated, the cells were washed in PBS and incubated for 72 hours in serum-free DMEM supplemented with various combinations of erythropoietin, dibutyryl cyclic AMP (dbcAMP), basic fibroblast growth factor (bFGF), heparin, 3-Isobutyl-1-methylxanthine (IBMX), PDGF-AA (platelet-derived growth factor), neuregulin-beta 1 (HRG-beta 1) (Uniprot Accession. No. Q7RTW4), epidermal growth factor (EGF), and 1× N-2 animal-free cell culture supplement (ThermoFisher Scientific, Cat. #1752048) was added. 100× N-2 contains 1 mM human transferrin (holo), 500 mg/L (milligrams per liter) Insulin Recombinant Full Chain, 0.63 mg/L progesterone, 10 mM putrescine, and 0.52 mg/L selenite. Conditioned medium (CM) from the cells was collected, and the cells were trypsinized and collected separately.

Analysis of BDNF concentration was performed using either an ELISA kit or a Luminex® kit.

Results

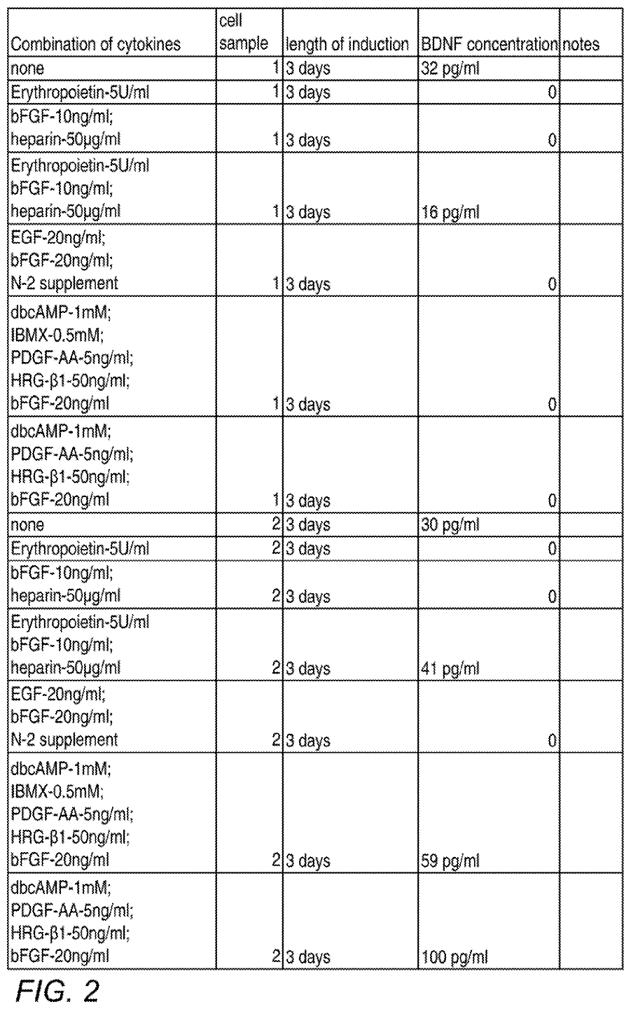

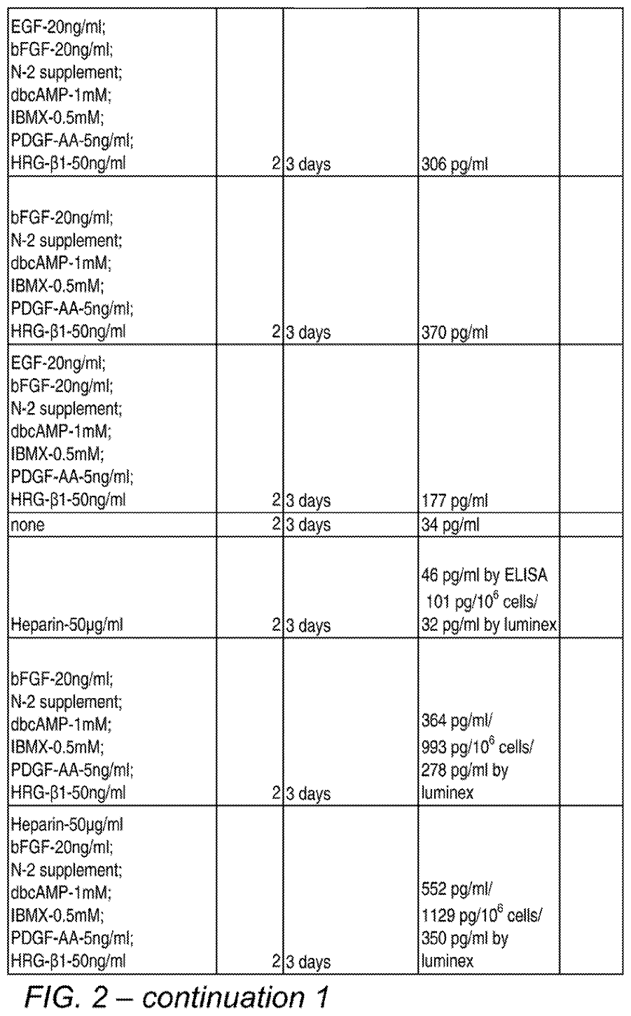

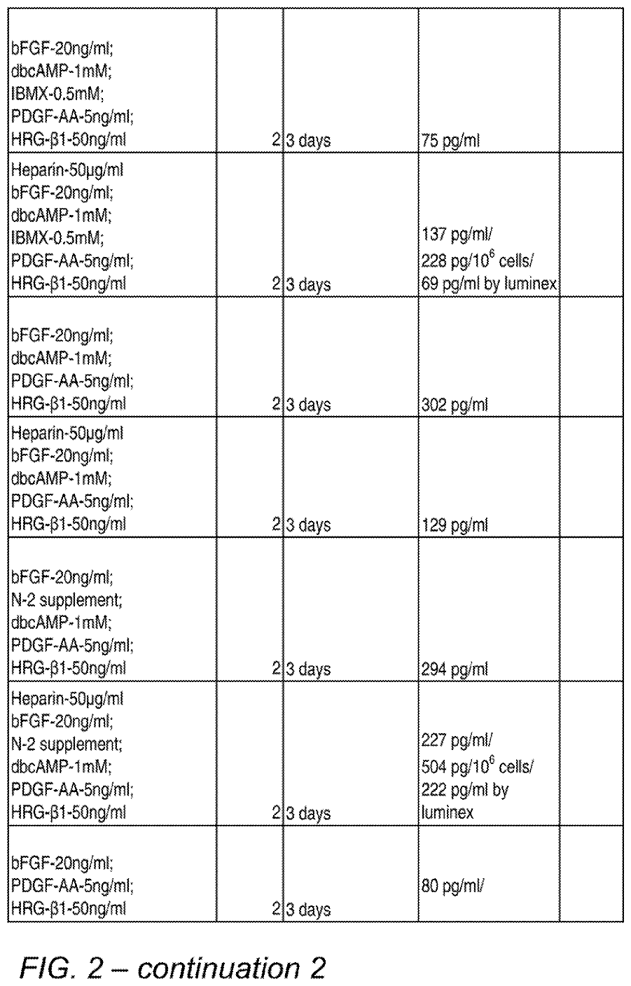

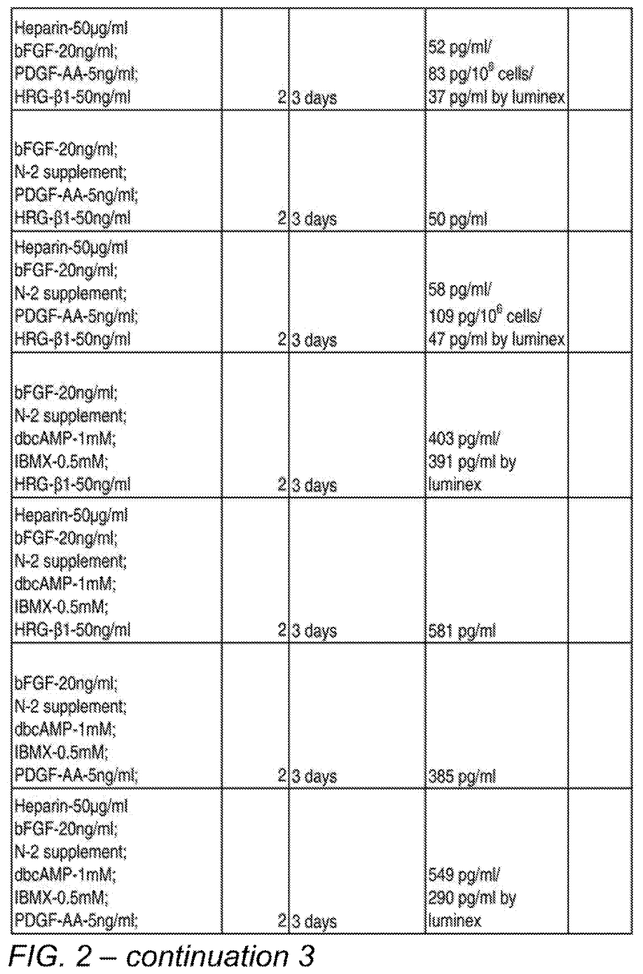

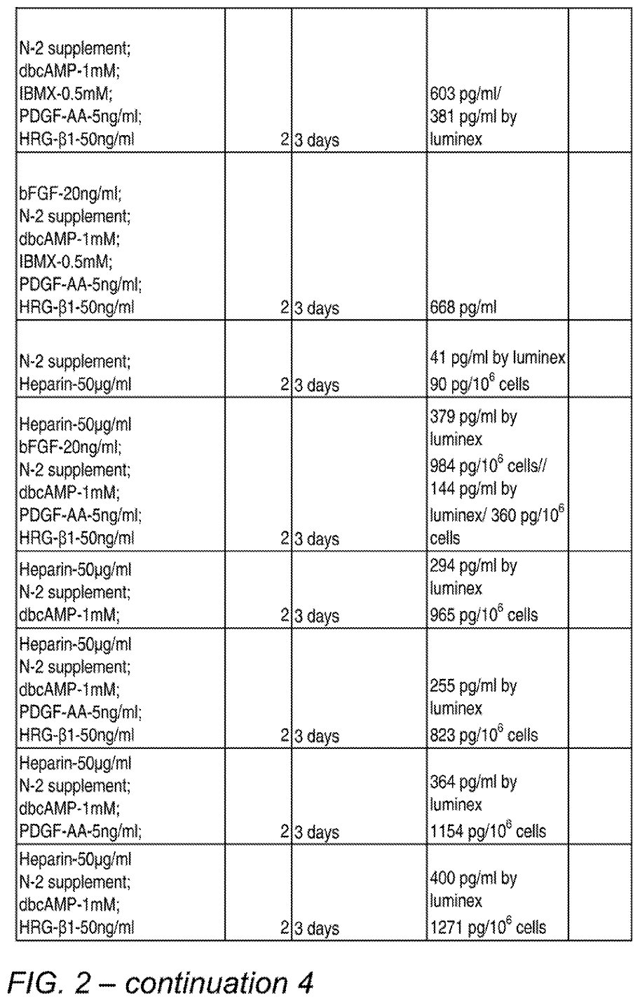

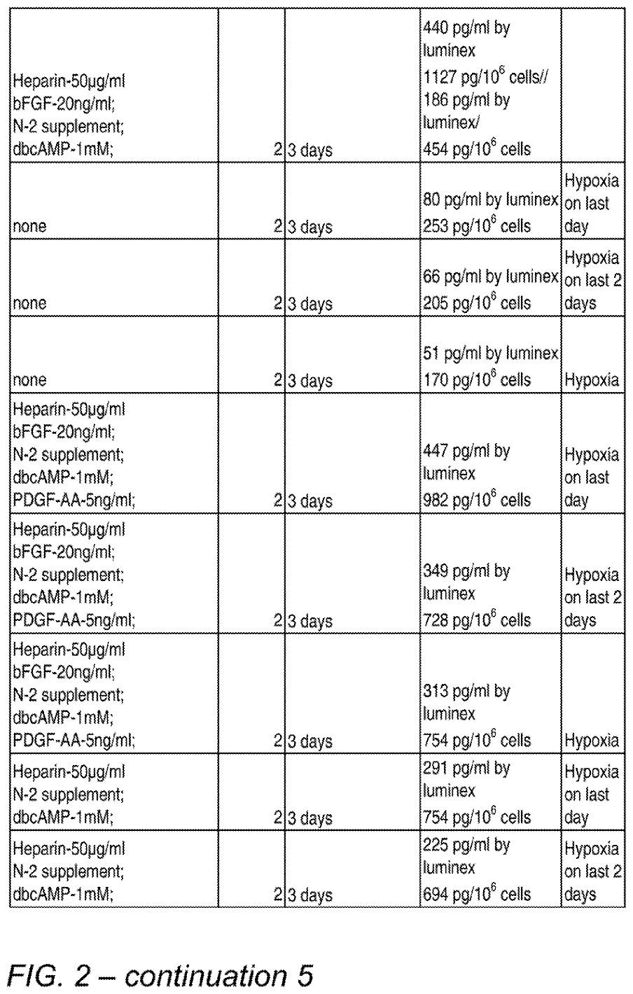

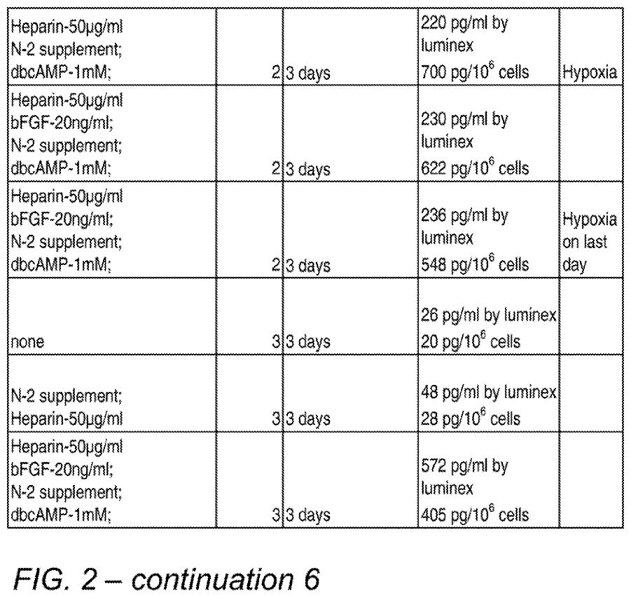

ASC were incubated in DMEM without serum supplemented with various combinations of erythropoietin, dbcAMP, bFGF, heparin, IBMX, PDGF, HRG-beta 1, EGF, and 1× N-2 supplement, in order to define the additives needed for induction. The CM was collected and analyzed for BDNF concentration (FIG. 2). Medium containing dbcAMP, heparin and N-2 supplement achieved induction after an incubation of only 3 days. Inclusion of bFGF further enhanced BDNF secretion and cell viability.

### Example 3: Further Characterization of Induced ASC

Methods

Cells were induced as described in the previous Example, using 1 mM (millimolar) dbcAMP, 20 ng/ml (nanograms per milliliter) bFGF, 50 mcg/ml (microgram/milliliter) heparin, and N-2 supplement to 1× concentration. The cells were incubated in the supplemented DMEM for 72 hours, after which the CM from the cells was collected. The cells were then trypsinized and collected separately.

The collected CM was analyzed for the presence and concentration of various factors using a commercial custom-made multiplex kit (Luminex®). Results are described for BDNF, G-CSF, IL-6, IL-8, LIF, VEGF-A, HGF and GDNF, since expression of these factors was affected by induction.

Results

Induction of 9 different batches of ASC for three days with dbcAMP, bFGF, heparin, and N-2 supplement significantly increased the levels of secretion of neurotrophic factors (BDNF, GDNF, VEGF, G-CSF [Granulocyte colony-stimulating factor receptor; Uniprot Accession No. Q99062], HGF, and LIF) and the immunomodulatory cytokines IL-6 and IL-8 (Uniprot Accession Nos. P05231 and P10145, respectively) in CM collected from ASC after induction (FIGS. 3A-C). Uniprot records in this paragraph were accessed on Oct. 29, 2017.

### Example 4: Induced ASC Continue to Secrete Neurotrophic Factors after Removal of Induction Agents

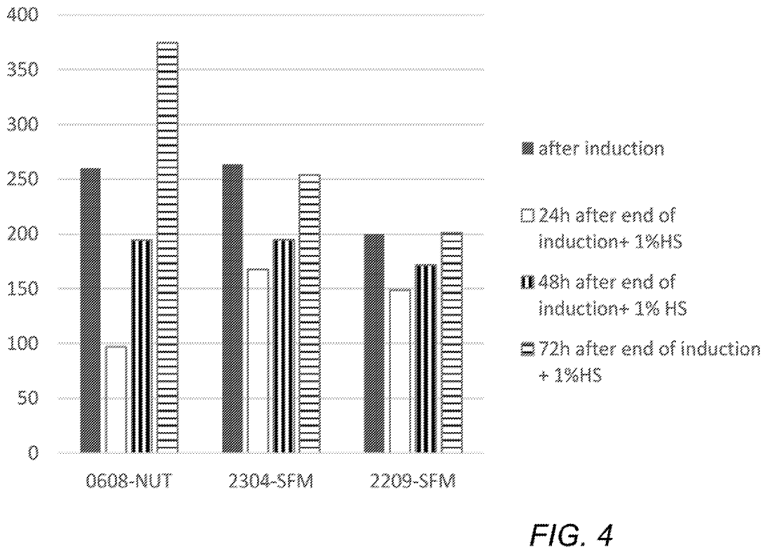

ASC were induced, as described in the previous Example, induction medium was aspirated, and cells were washed and incubated in medium containing 1% human serum (HS) for 72 hours, taking a sample of CM every 24 hrs. BDNF levels were measured and compared to levels in CM collected immediately after induction. Even 72 hours after the conclusion of induction, the cells continued to secrete BDNF at a similar rate to those immediately after induction (FIG. 4), showing that the cells' secretion of neurotrophic factors was sustained.

### Example 5: Enhancement by Serum of ASC Induction

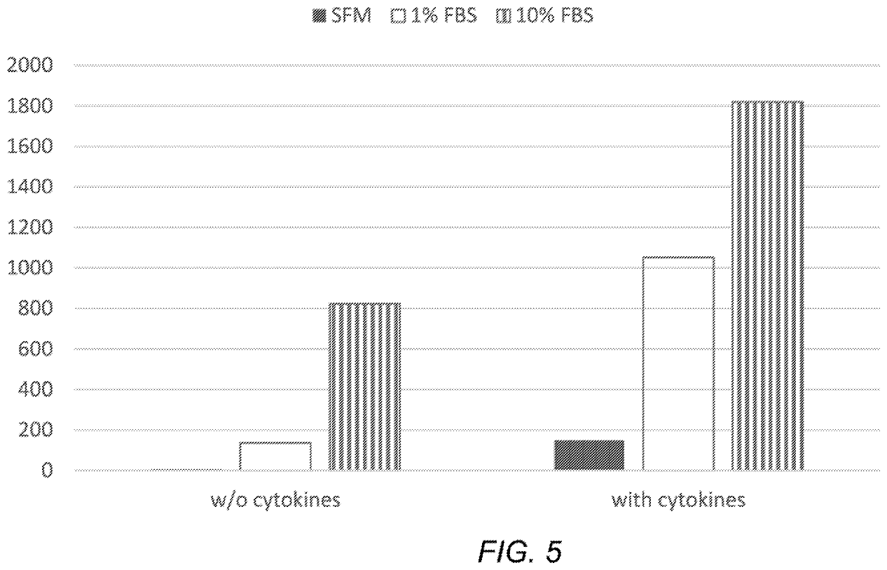

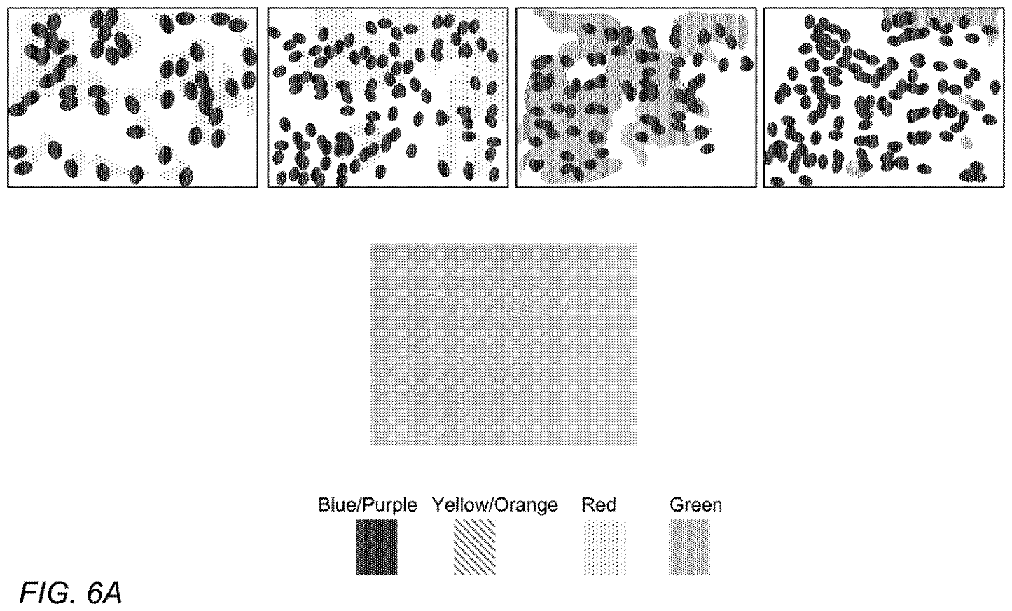

ASC were induced, as described in Example 3, except for inclusion of 1% or 10% FBS in some samples. At the conclusion of induction, CM was collected. Serum significantly enhanced BDNF secretion in a dose-dependent manner even in the absence of other induction agents (FIG. 5).

### Example 6: Cm from Induced ASC Stimulates Differentiation of Neuronal Precursor Cells

Methods

SH-SY5Y cells were incubated with regular SH-SY5Y growth medium (composed of 50% MEM medium supplemented with non-essential amino acids; 50% F-12; 10% FBS; 1% glutamine; 0.5% sodium pyruvate and 50 μg/ml gentamycin) or CM from ASC that were induced as described in Example 3 (prepared in SH-SY5Y growth medium instead of DMEM), to elicit differentiation for 6 days. Medium was replaced after 3 days.

IHC Staining.

Cells were then fixed, permeabilized, and stained with antibodies against either human Nestin, human βIII-tubulin, human Choline acetyl transferase, or human Tyrosine hydroxylase (Abcam) followed by fluorescent labeled secondary antibodies (either Alexa Fluor®488 for Choline acetyl transferase and Tyrosine hydroxylase or CF™543 for Nestin and βIII-tubulin). Nuclei were stained with DAPI.

Samples were viewed with an Olympus BX53 fluorescent microscope and the CellSens program was used to take pictures, and analyze neurite length.

Results

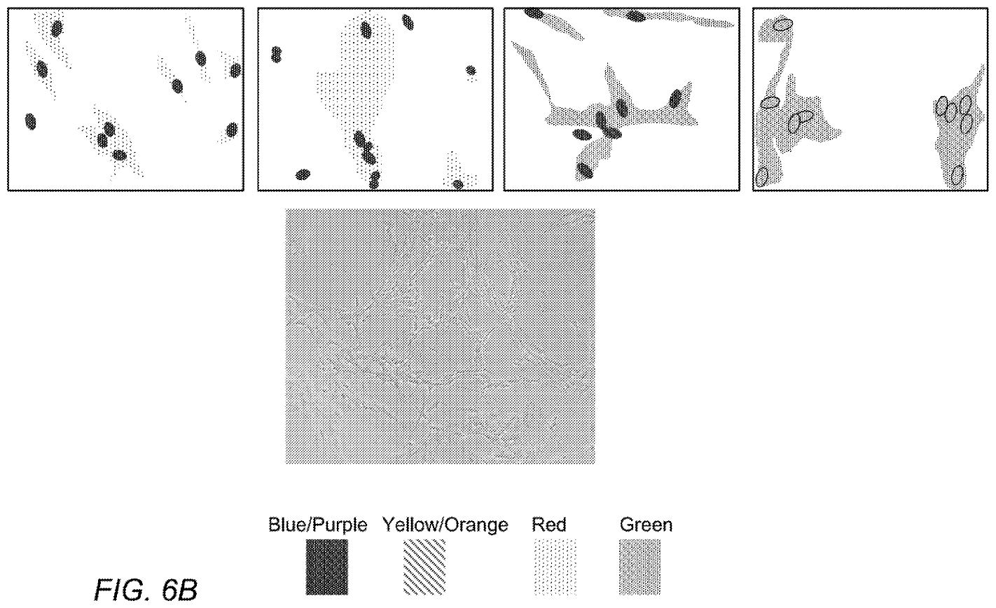

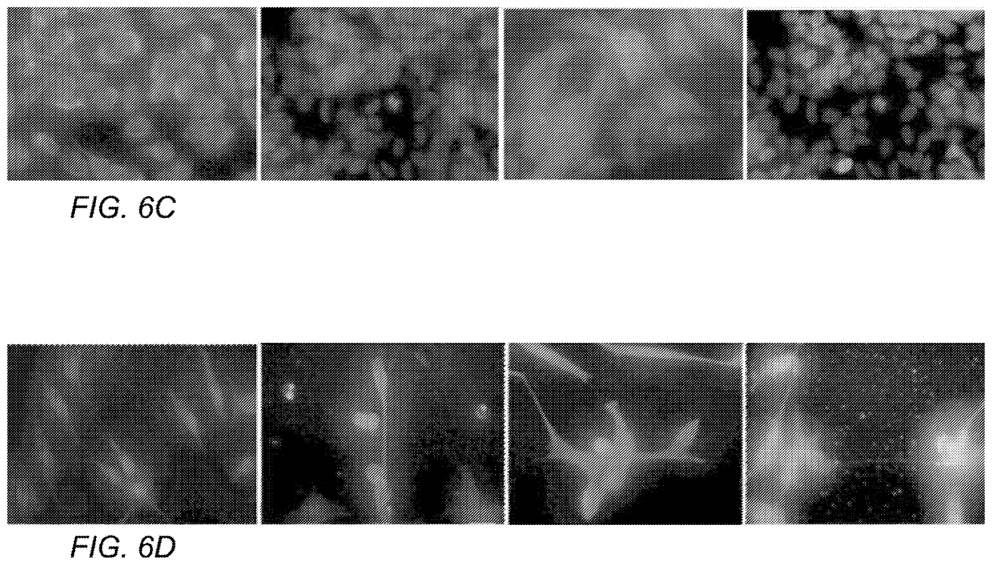

SH-SY5Y cells are undifferentiated human neuroblastoma derived cells that express immature neuronal markers and lack mature neuronal markers. SH-SY5Y cells were incubated with regular SH-SY5Y growth medium or CM from induced ASC (“induced CM”). Exposure to induced CM stimulated the differentiation of SH-SY5Y cells, as evidenced by a significant morphological change, with the production of long neurites extending from the cells. This was noticeable as soon as 24 hours after exposure to the CM. After six days of exposure to induced CM, a significant change in protein expression could be detected. The expression of the neural precursor marker Nestin decreased, while there was a dramatic increase in the expression of the mature neuronal marker β-III-tubulin, mainly detected in the extending neurites, and other markers, as shown by immuno-histochemical (IHC) staining. Expressed markers included both Choline acetyl transferase (indicative of a cholinergic phenotype) and Tyrosine hydroxylase (indicative of dopaminergic or noradrenergic potential). This effect was not seen with SH-SY5Y cells incubated in SH-SY5Y growth medium. Results are shown in FIG. 6B, SH-SY5Y exposed to induced CM; in comparison to FIG. 6A, control cells.

A quantitative analysis of the results confirmed that exposure to induced CM caused differentiation, as indicated by at least one long neurite, in 44.4±9.5% of SH-SY5Y cells in contrast to SH-SY5Y growth medium, which elicited neurite outgrowth in less than 10% of cells. Neurite length measurements indicated that induced CM gave rise to significantly longer neurites than control CM.

As expected, the differentiated cells stopped proliferating, as indicated by a lack of increase in the amounts of cellular DNA detected using the DNA binding dye-Cyquant. Although the cells did not proliferate further, there was no secretion of lactate dehydrogenase, indicating that cellular viability was retained.

### Example 7: Additional ASC Induction Protocols

To determine the best conditions for induction, ASC were thawed at p3/3 and were seeded in 175 cm² flasks and grown for 5 days in DMEM+20% FBS, in some cases in the presence of induction agents for the last 24 or 72 hours (induction agents were added either in the presence of DMEM+20% or DMEM without FBS). Induction was similar to Example 3, but with various parameters altered and various initial seeding densities. CM was collected after no induction or 24- or 72-hr. of induction (see Table 3) and tested for BDNF concentration.

Additionally, aliquots of cells were cryopreserved at the conclusion of the induction and were subsequently thawed, seeded in 6-well plates at 0.5×10\hat{ }6 cells/well, and incubated for 72 hr. in DMEM+20% FBS, withdrawing a sample of CM every 24 hrs, which was tested for BDNF concentration. At 72 hours, cells were removed from the plates and counted for normalization.

TABLE 3
Induction Conditions for Samples.

| | Cell no./cm\hat{ }2 at | | | |
|---|---|---|---|---|
| Group | time of seeding | Induction time (hr) | Agents present? | Medium |
| 1 | 2300 = | (none) | − | DMEM + 20% |
| 2 | 0.4 × 10\hat{ }6 cells | 24 | + | FBS |
| 3 | | 72 | + | |
| 4 | 4600 = | (none) | − | |
| 5 | 0.8 × 10\hat{ }6 cells | 24 | + | |
| 6 | | 72 | + | |
| 7 | 17,000 = | (none) | − | |
| 8 | 2.9 × 10\hat{ }6 cells | 72 | + | Basal DMEM |
| | (previous | | | |
| | conditions) | | | |

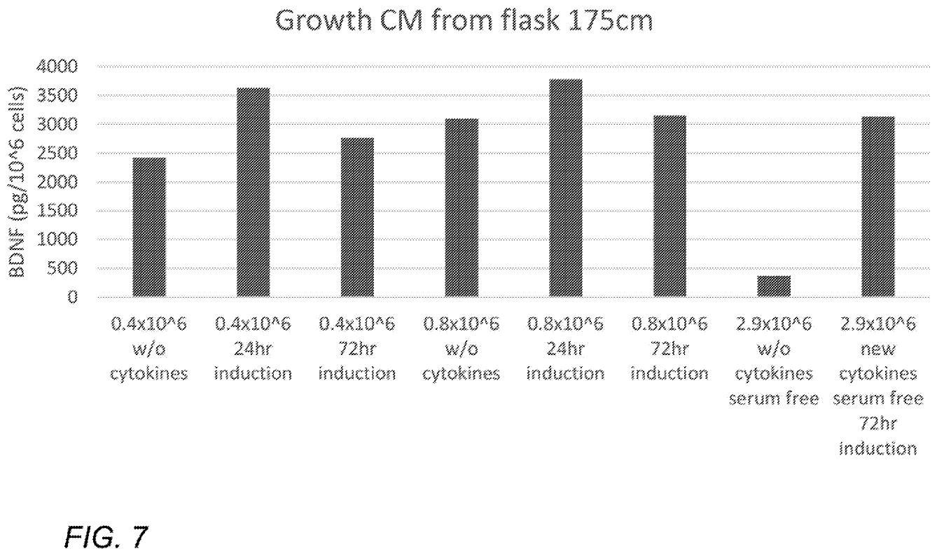

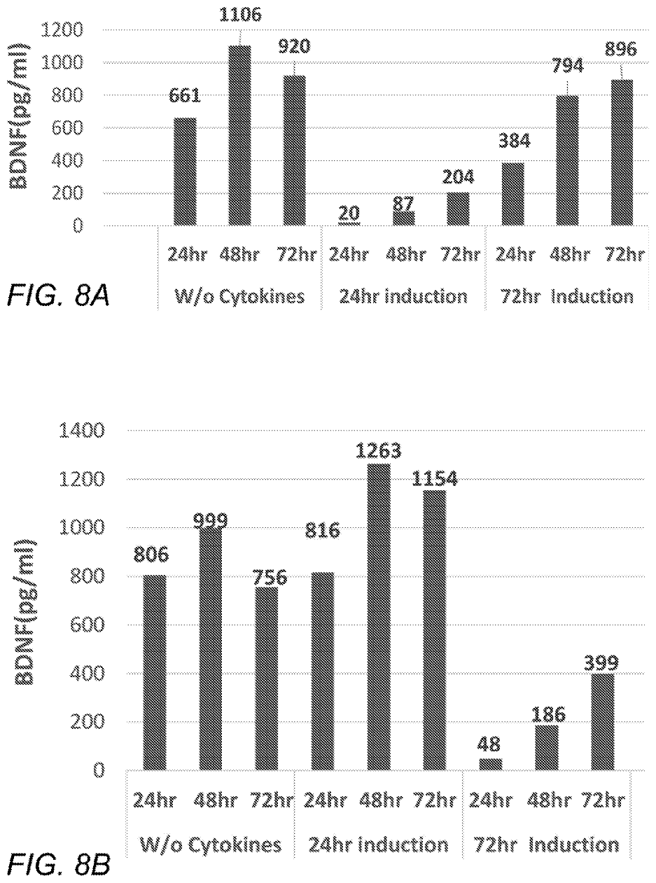

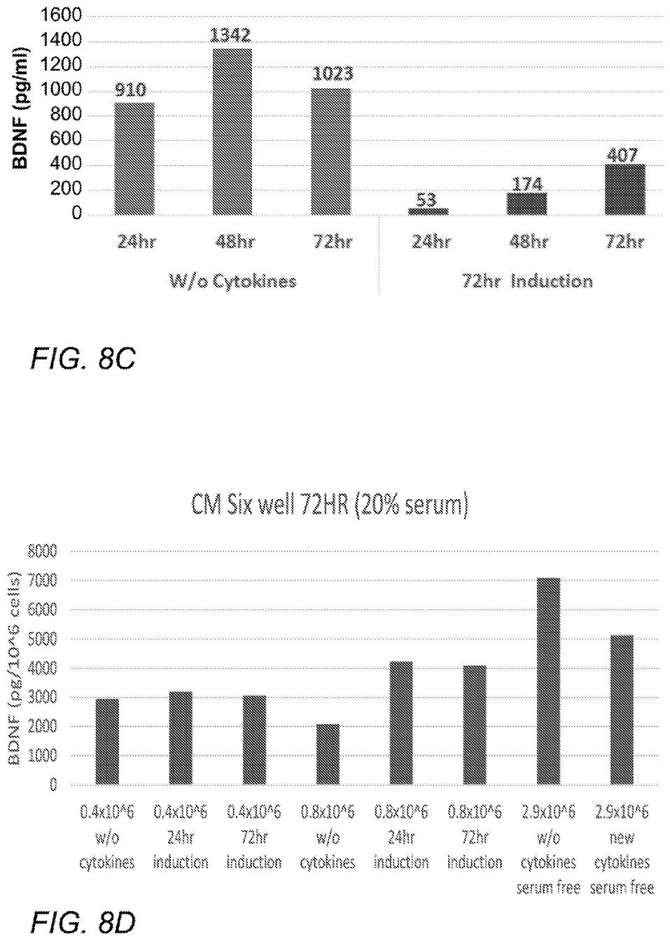

Measurement of BDNF concentration in CM at the conclusion of the induction showed that seeding density did not affect BDNF secretion, and induction agents had a relatively small additive effect in the presence of serum, and a larger effect under serum-free conditions (FIG. 7; rightmost 2 bars depict serum-free conditions in the absence of presence of induction agents). Aliquots of cells were also cryopreserved after induction, and BDNF secretion was measured after thawing, which is shown for the low-, medium-, and high density groups (FIGS. 8A-C, respectively). BDNF secretion appeared lower after 72-hr. induction in the high-density/serum free group compared to 24 hr. induction in the medium-density group when looking at absolute numbers (compare FIG. 8C, rightmost 3 bars to FIG. 8B, middle set of 3 bars). However, when numbers were normalized to the number of cells, BDNF levels in the high-density/serum free group were similar to the low and medium density groups (FIG. 8D; compare the 1^{st} and 4^{th} bars from the right). This was due to the low cell viability in this group. This showed that use of serum-containing medium for induction enabled cells to better recuperate from cryopreservation and thus the number of viable cells secreting BDNF is higher, hence the higher absolute concentrations observed.

In further experiments, 0.8×10\hat{ }6 cells per 175 cm² flask were seeded and induced with bFGF, dbcAMP, heparin and 1× N-2 supplement for 24 hr. in DMEM+20% FBS. Post-cryopreservation and thawing, CM was collected after incubating cells for 24 hr. in DMEM+20% FBS. Measurement of various cytokines confirmed that 24-hr. induction was in most cases even more effective than 72-hr induction (FIG. 9A-C).

Conditions of 4600 cells/cm\hat{ }2 (0.8×10\hat{ }6 cells per 175 cm² flask) and 24-hr. induction time were used for further experiments.

### Example 8: Comparison of Bioreactor-Expanded ASC to 2D-Induced ASC

Methods

Induction Agent-Induced ASC were produced by seeding passage 3/3 ASC into flasks at 4600 cells/cm\hat{ }2 and incubating them for 5 days in DMEM+20% FBS. Next, medium was exchanged, and cells were incubated for an additional 24 hr. in DMEM+20% FBS, with either no induction agents or regular or high concentrations of induction agents. Cells were cryopreserved, thawed, and incubated for 48 hours (24 hours in DMEM+20% FBS, followed by 24 h in serum-free SH-SY5Y medium), after which CM was collected. Regular concentrations are described in Example 3, while high concentration medium contained increased concentrations of bFGF (100 ng/ml) and N-2 supplement (diluted 1:20 instead of 1:100).

Bioreactor-expanded ASC were produced as described in Examples 1-2 until passage 3/3. Cells were then trypsinized. 170×10⁶ cells were seeded into each 2.8-liter bioreactor, which contained New Brunswick Scientific FibraCel® carriers made of polyester and polypropylene and culture medium (DMEM+20% FBS). Cells were maintained at: temp: 37±1° C., Dissolved Oxygen (DO): 70±10% and pH 7.4±0.2. Filtered gases (Air, CO₂, N₂ and O₂) were supplied as determined by the control system in order to maintain the target DO and pH values.

After seeding, the medium was agitated with stepwise increases in the speed, up to 150-200 RPM by 24 hours. Perfusion was initiated several hours after seeding and was adjusted on a daily basis in order to keep the glucose concentration constant at approximately 550 mg\liter.

Cells were typically harvested after 5-6 days by washing the cells, adding trypsin, and subjecting them to agitation.

Downstream Processing: Cell Concentration, Washing, Formulation, Filling and Cryopreservation

Prior to assaying, cells were suspended and washed in suspension solution (5% w/v human serum albumin [HSA] in isotonic solution), then adjusted to 10-20×10⁶ cells/ml, in isotonic solution with 10% DMSO v/v and 5% HSA w/v. The vials were gradually chilled and stored in a gas-phase liquid nitrogen freezer.

Results

ASC were incubated in the presence of no induction agents (negative control) or induced with regular or high concentrations of induction agents. Afterwards, cells were cryopreserved, thawed, and seeded at 0.5*10\hat{ }6 cells/well in DMEM+10% FBS. The following day cells were washed with PBS and incubated for an additional 24 h. in serum-free DMEM, serum-free SH-SY5Y growth medium or SH-SY5Y growth medium+10% FBS, from which CM was collected.

In parallel, the same batch of cells was expanded in DMEM+10% FBS in a bioreactor (instead of incubation with induction agents), for comparison of cytokine secretions. Cryopreserved cells were thawed and seeded for CM collection, as described in the previous paragraph.

As before, incubation with induction agents (at regular or high concentrations) induced secretion of various cytokines, compared with 2D-cultured cells without induction agents. Bioreactor-expanded ASC had a cytokine profile that was similar to, but distinct from, induction-agent induced ASC (FIGS. 10A-C).

### Example 9: Neuronal Differentiation by Cm Derived from ASC Bioreactor Expanded or Incubated with Inducing Agents

Methods

ASC were incubated with regular concentrations of induction agents, as described in the previous Example, then were cryopreserved and thawed, and CM was collected as described above, but in SH-SY5Y growth medium (MEM/F12) with 10% FBS for the last 24 hr. (CM was also collected from induced ASC in the absence of serum; this yielded similar but less pronounced neuronal differentiation). SH-SY5Y growth medium contains 50% MEM medium supplemented with non-essential amino acids; 50% F-12; 10% FBS; 1% glutamine; 0.5% sodium pyruvate and 50 μg/ml gentamycin.

CM was also collected from bioreactor-expanded ASC as in the previous Example.

For differentiation assays, SH-SY5Y cells were incubated for 6 days with regular SH-SY5Y growth medium, CM from uninduced or induced ASC, or regular SH-SY5Y growth medium supplemented with butyric acid (positive control), to elicit differentiation. Medium was refreshed after 3 days.

IHC Staining.

Cells were then fixed, permeabilized, and stained with antibodies against human βIII-tubulin and human tyrosine hydroxylase (Abcam), followed by fluorescent labeled secondary antibodies (either Alexa Fluor®488 for tyrosine hydroxylase or CF™ 543 for βIII tubulin). Nuclei were stained with DAPI.

Samples were viewed with an Olympus BX53 fluorescent microscope and the CellSens program was used to take pictures, and analyze neurite length.

Results

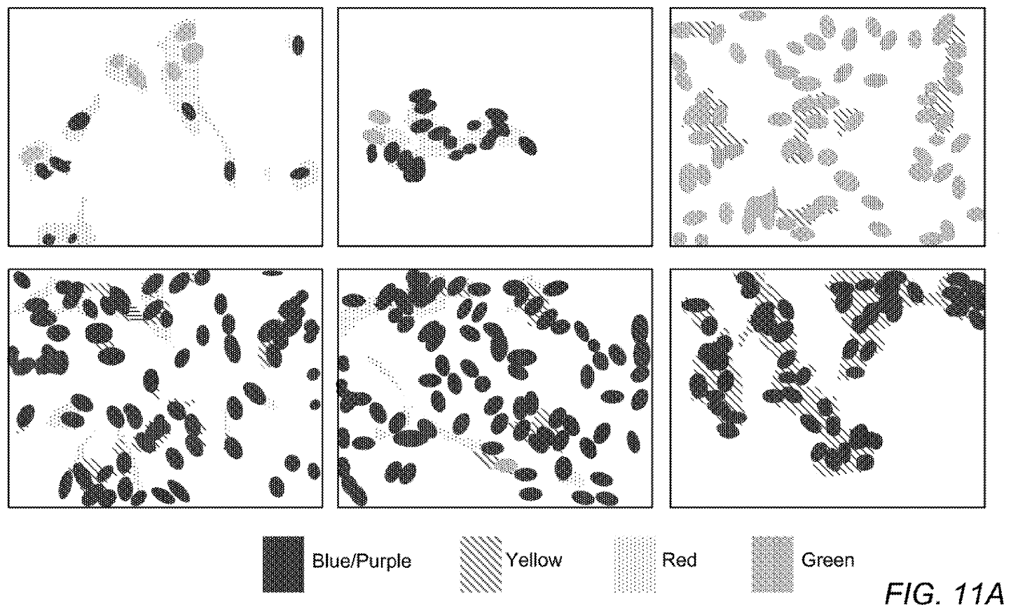

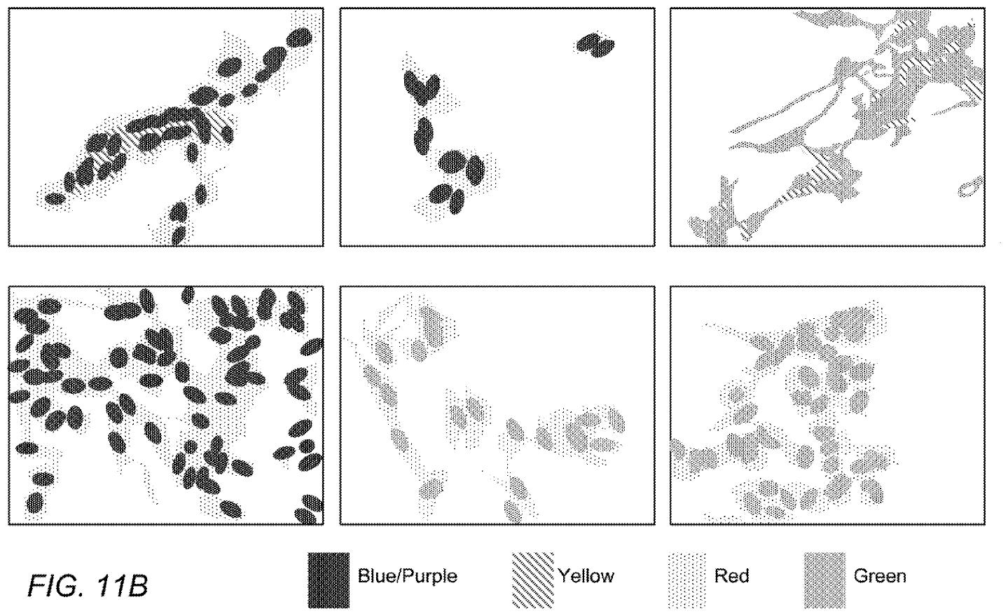

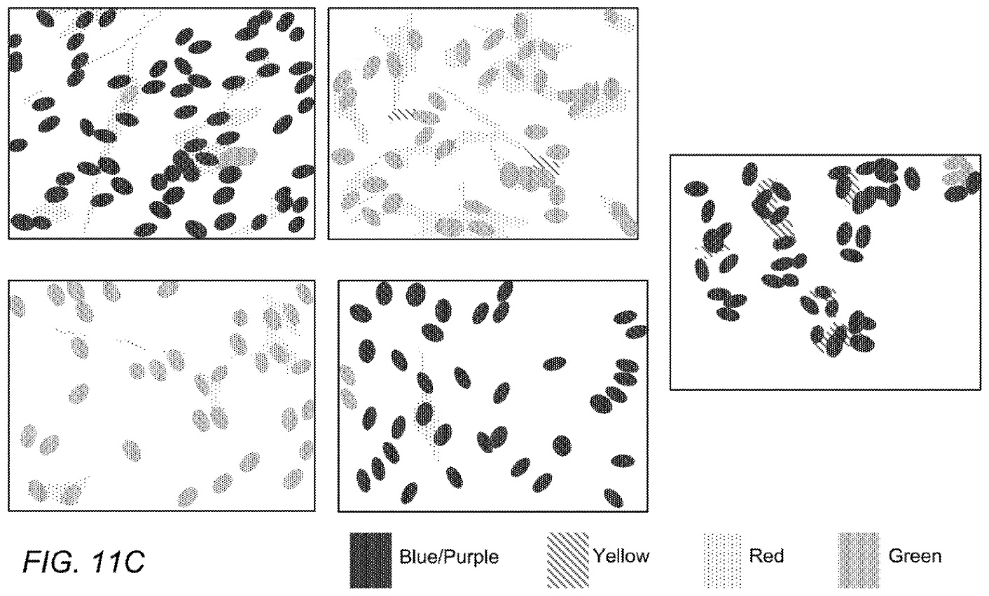

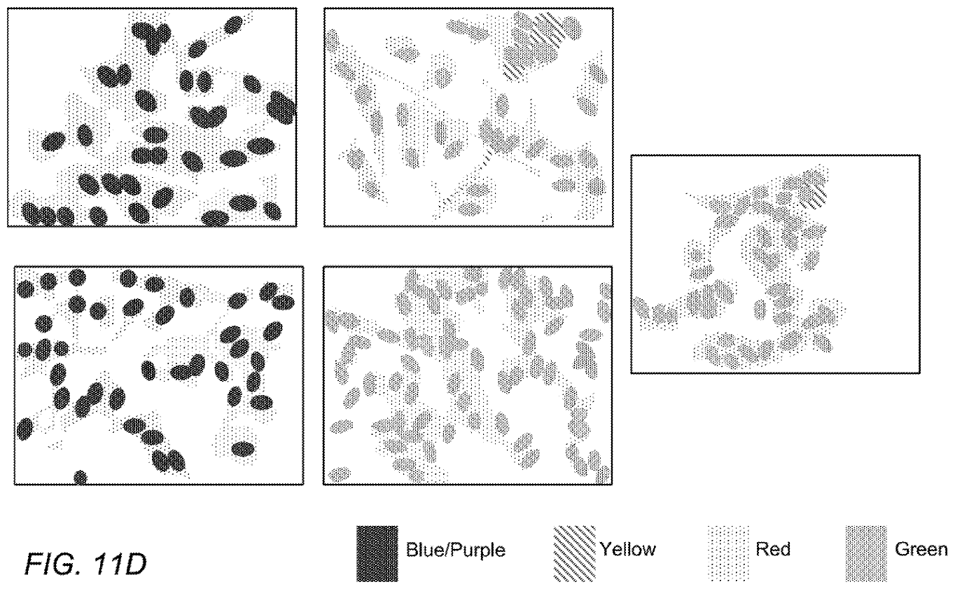

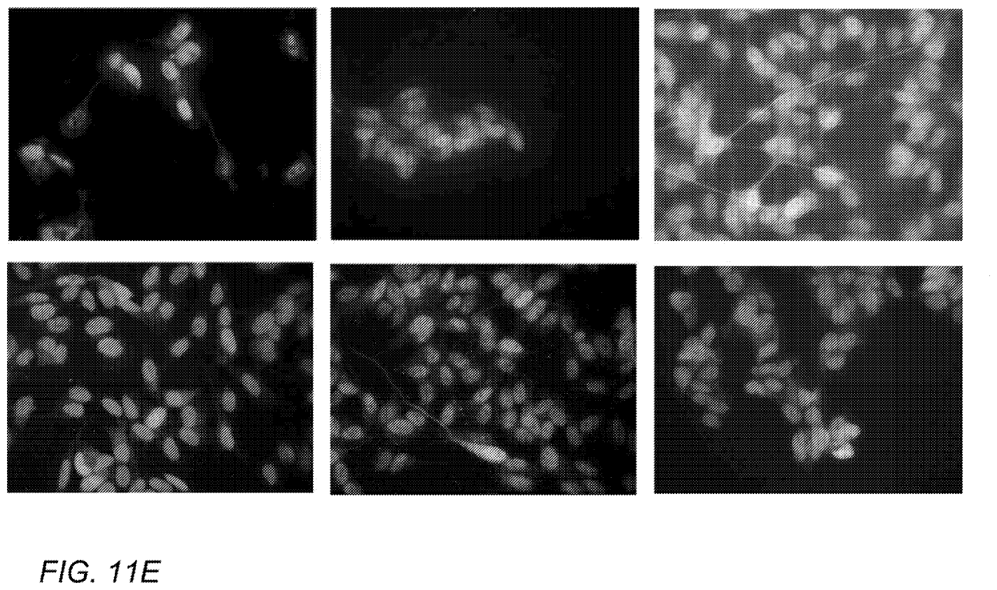

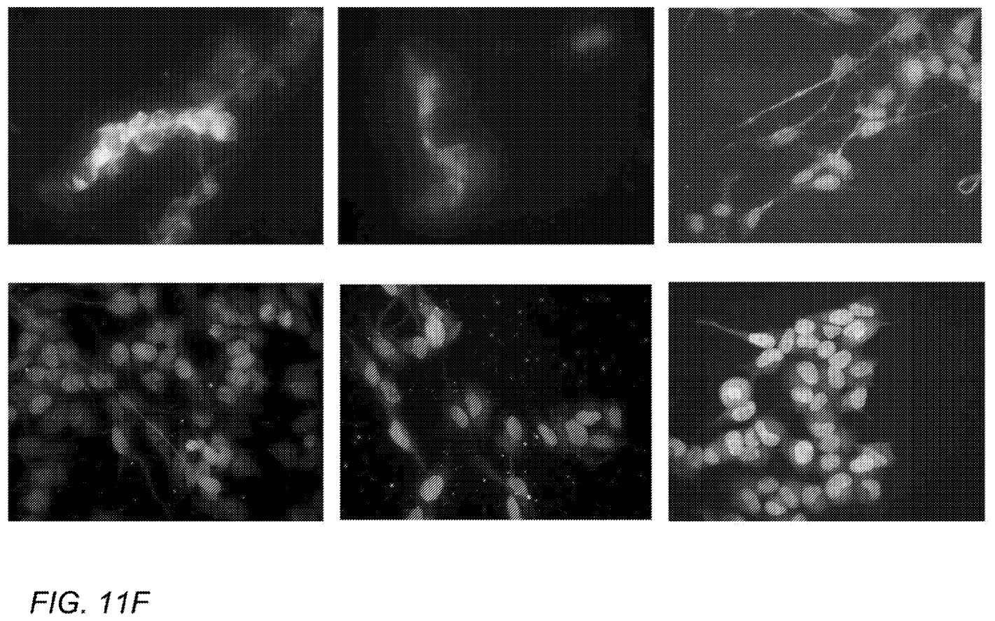

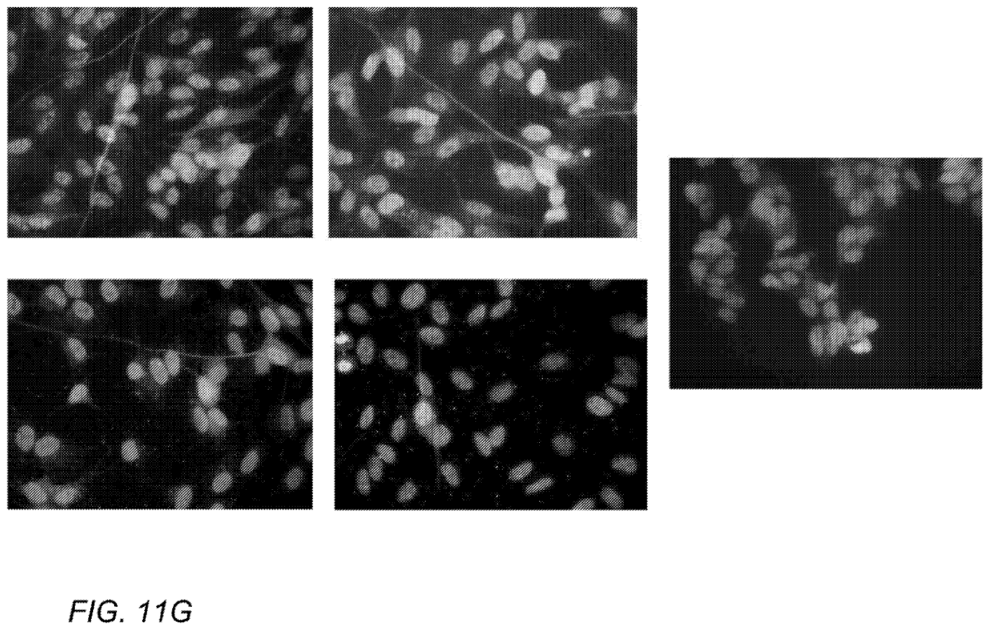

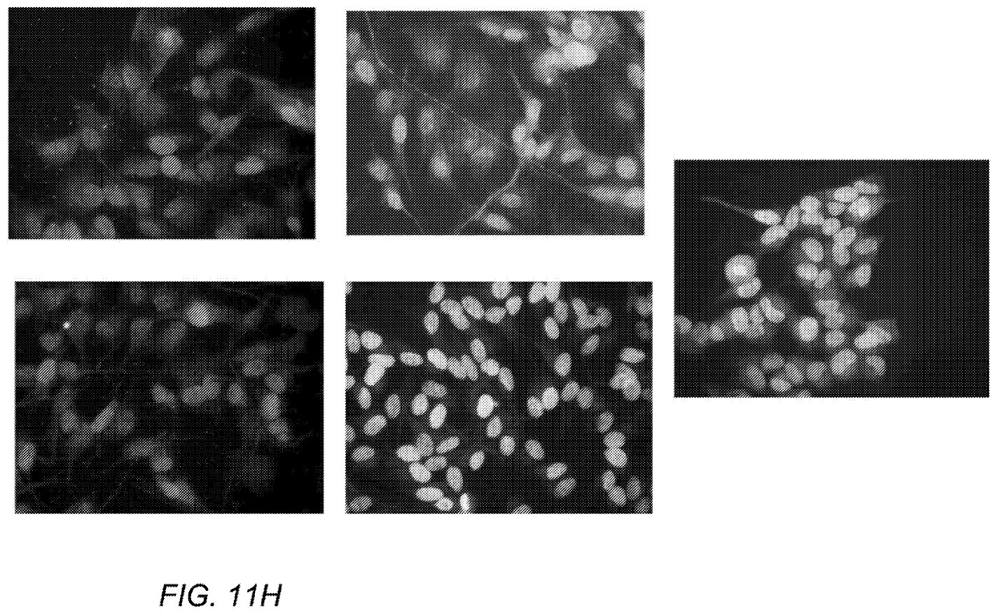

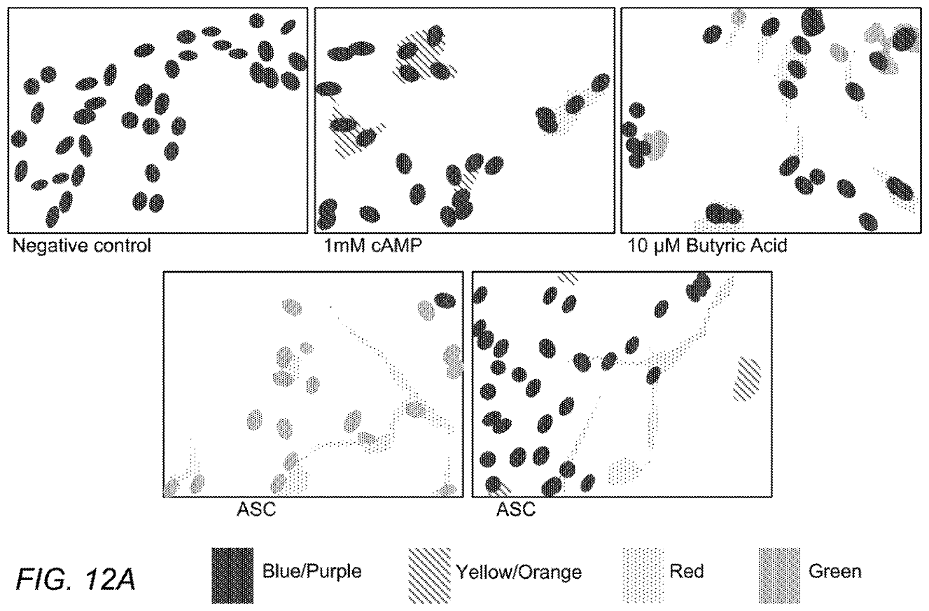

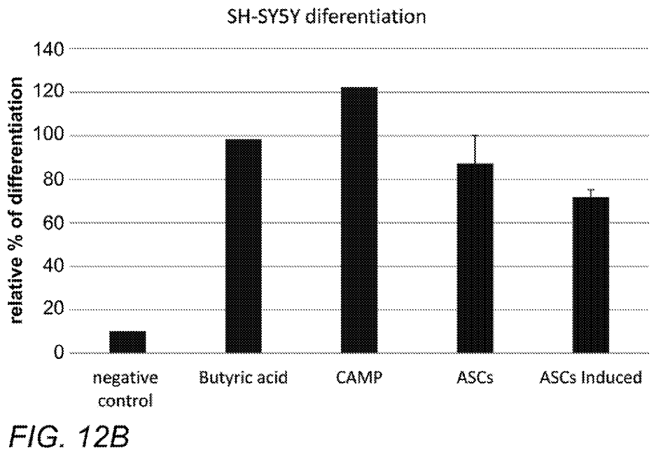

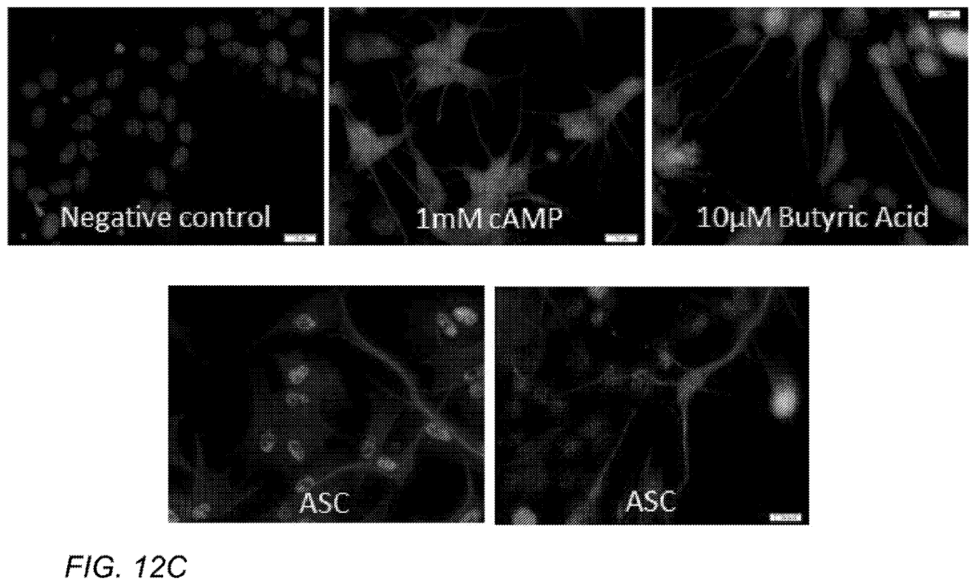

SH-SY5Y cells were incubated with CM from ASC incubated with regular concentrations of induction agents, or bioreactor-expanded ASC, to determine their ability to induce neuronal differentiation. Certain batches of bioreactor-expanded ASC induced neuronal differentiation as shown by upregulation of beta-III-tubulin expression, a mature neuronal marker, and neurite elongation. High TH and ChAT expression were induced, indicating differentiation of neuronal precursor cells into dopaminergic and cholinergic or noradrenergic neurons (FIGS. 11A-B, upper right panels; FIG. 12A, bottom panels; compare to positive control butyric acid [12A, upper right panel]).

Additionally, CM from certain batches of ASC grown in tissue culture plates caused neuronal differentiation, as evidenced by increased tubulin expression (FIG. 11C, top left and top middle panels). CM from induction agent-incubated ASC had the additional effect of inducing neuronal precursor cells to reduce Nestin expression and increase their TH and ChAT expression, indicating differentiation into dopaminergic and cholinergic neurons (FIG. 11D, top middle panel).

FIG. 12B shows the quantitation of neuronal differentiation by the various CM tested, calculated as the percent of cells with extended neurites that stained for beta-III-tubulin relative to the butyric acid positive control.

### Example 10: ASC Induction in a Bioreactor Setting

Bioreactor-expanded ASC are produced as described in Example 8, except that induction agents are added to the medium on the last day of the bioreactor incubation. Following cryopreservation and thawing, ASC are plated, and CM is collected. CM is assessed for the presence of neurotrophic factors and the ability to induce neuronal differentiation.

### Example 11: Intranasally Injected ASC Migrate to the Brain

Methods

Gold nanoparticles (GNPs) are described in Betzer et al.

Mice were injected either intra-nasally (5×10⁵ cells) or IV (1×10⁶ cells) with GNP stained ASC. 24 hours after injection, mice were sacrificed, and the whole body was scanned for GNP stained cells using a microCT imager.

Results

ASC were able to be stained with gold nanoparticles (GNPs) (Betzer et al) with only minimal effects on cell viability (as indicated by the percentage of plastic-adherent cells within 6 hours of incubation and no reduction of cell functionality (as indicated by endothelial cell proliferation and bone marrow migration).

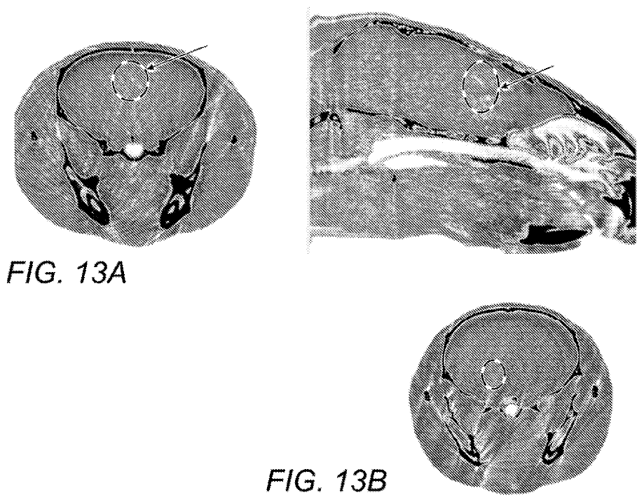

GNP-stained ASC were tracked for 24 hours after injection into mice either IV or intranasally. Twenty four hours after intranasal injection, CT imaging detected a large number of ASC in the brain (seen as green dots—FIG. 13A) while much fewer cells were detected after IV injection (FIG. 13B). GNPs that were not incubated with cells served as a negative control, since GNPs alone cannot cross the blood-brain barrier under the conditions utilized. These results show that intranasal injection is a viable route for administration of ASC to the brain.

### Example 12: Induced ASC Reduce ROS Production and Resultant Cell Mortality

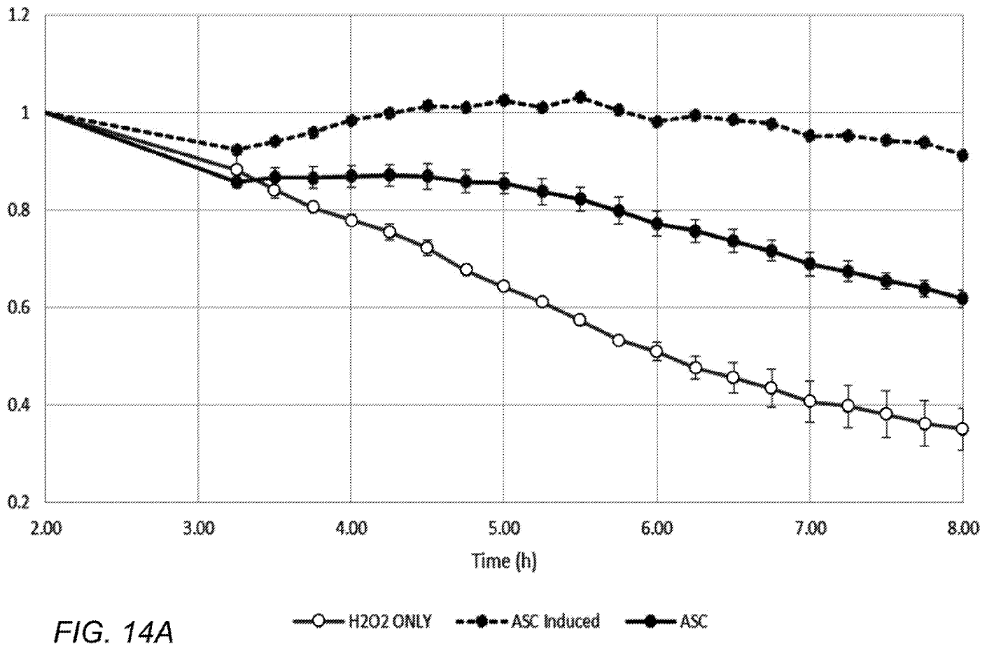

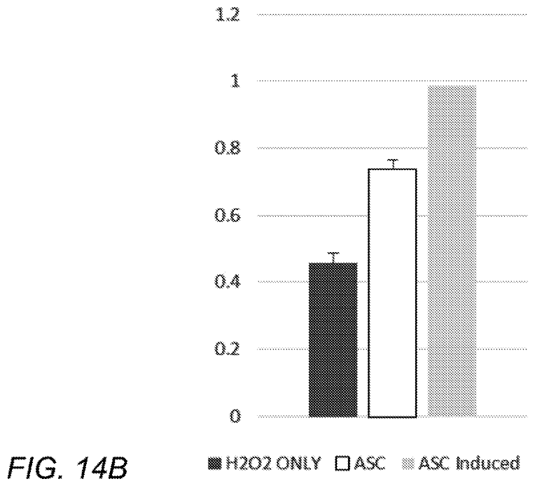

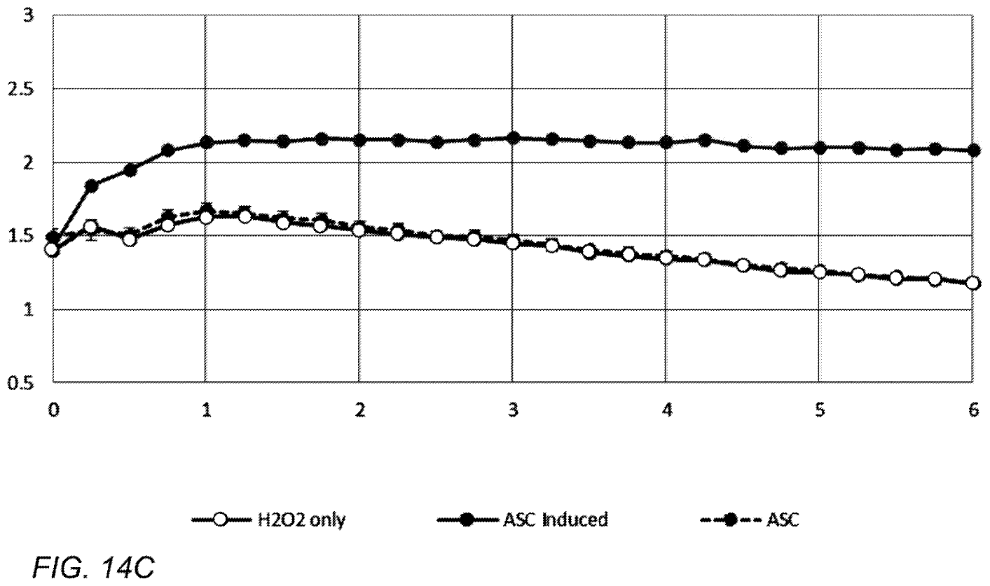

An assay was developed to determine the cytoprotective/antioxidant effects of placental ASC subjected to bioreactor expansion or incubation with inducing agents. Neuroblastoma (SH-SY5Y) cells were differentiated using cAMP for 7 days, to recapitulate the effect of oxidative stress on fully differentiated neurons in vivo. Following neuronal differentiation, cells were incubated in regular growth medium (control), or CM derived from placental ASC subjected to bioreactor expansion or incubation with inducing agents, in the presence of RealTime GLO™ (RTG) reagents, which detect viable cells. The cells were incubated with the RTG reagents for 2 hours, enabling the RTG to enter the cells and equilibrate. Then H₂O₂ was added to the cells, and luminescence values, correlating with live cell number, were measured every 15 minutes for 8 hours. FIG. 14A shows that cells exposed to H₂O₂ in control medium (solid, gray line) exhibited increased cell death relative to controls without H₂O₂, while CM from placental bioreactor-expanded ASC (solid, black line) or, even more so, following incubation with bFGF and cAMP (dotted line) conferred a significantly higher cell viability. The peak difference was seen 6.5 hours following addition of H₂O₂ (FIG. 14B).

Furthermore, formation of ROS was measured using dichlorofluorescin diacetate (DCFDA), a fluorogenic dye that measures intracellular ROS activity within the cell. DCFDA was added to differentiated SH-SY5Y cells for 45 minutes, residual dye was washed away, then CM or control medium (groups labeled as in FIG. 14A) with H₂O₂ were added to the cells. ROS activity was determined every 15 minutes for 6 hours. H₂O₂ caused an increase in ROS activity, as expected, but CM inhibited ROS formation (FIG. 14C). The 2 types of CM behaved similarly to one another.

### Example 13: Use of ASC in Treating ALS

Overview

An experiment was performed to evaluate the effect of ASC vs. placebo treatment (intramuscular [IM]+intrathecal [IT]) on the life span and neurological and motor impairments in SOD1^{G93A} ALS mice.

Experimental Details

Overall Study Design:

36 SOD1^{G93A} transgenic familial ALS mice (high copy number; B6SJL, Hemizygous for Tg (SOD1*G93A) 1 Gur/J) (Gurney, 1997) ˜6 weeks old and weighing ˜20-25 g were used. Mice were weighed 3 times per week throughout the experiment.

At the earlier timepoints, bioreactor-expanded ASC were administered twice, by the IM and IT routes at each treatment. The first treatment was given when initial symptoms of disease onset appeared, (as measured by decline from peak average weight), and the second treatment was given 1 week after the first treatment and was similar. Placebo-injected mice served as a negative control.

Motor strength was assessed by the strength grip and Rotarod assessments. Motor function was scored on a scale of 0-5.

Animals were euthanized when they were unable to get up from a lying position within 30 seconds.

Additionally, one month after the beginning of treatment, 9 surviving mice from the placebo group that had a score below 4 and had lost less than 15% of their peak weight were divided into two groups. 4 mice received an IT injection of 0.5 million bioreactor-expanded ASC (“late treatment”), and 5 mice received a 3rd placebo injection.

Behavioral Analyses:

Behavioral experiments were conducted during the light cycle by a researcher blinded to the treatment group. Mice were trained in the Rotarod and grip strength tests.

Rotarod Performance Test.

Training sessions were conducted to allow the mice to adapt to the Rotarod apparatus (Columbus Instruments, Columbus, Ohio, USA). Fore- and hind limb motor coordination and balance were assessed by measuring the time that the mice remained on the rotating rod (gradually increasing the speed from 4 to 40 rpm within a cut-off of 300 s) (Azzouz et al., 2000). Two trials were conducted with each mouse, separated by 10-min inter-trial intervals, and the longest retention time (maximum of 300 s) was recorded.

Grip Strength Assay.

This test used a Grip Strength Meter 47200 (Ugo Basile, Varese, Italy) to measure grip-strength (peak force and time of resistance). Mice were placed over a base plate, in front of a grasping bar, that was fitted to a force sensor connected to the peak amplifier, enabling reliable and automated detection of the response. Grip force and length of time holding onto the bar were documented.

Basso, Beattie, Bresnahan (BBB) Locomotor Rating Scale.

The BBB score was used to assess locomotor testing (Basso et al, 1995). The scale (0-5) used reflected hindlimb movements, stepping, forelimb and hindlimb coordination, trunk position and stability, paw placement and tail position, as follows:
0—Normal motor function
1—Tail weakness.
2—Weakness of one hind limb.
2.5—Paralysis of one hind limb, with the other limb other still functioning
3—Weakness of both hind limbs
3.5—Paralysis of one hind limb; other limb exhibits weakness
4—Permanent paralysis of the hind limbs, the front limbs still functioning.
5—Permanent paralysis of the hind limbs; front limbs exhibit weakness and shivering.

Determination of Disease Onset:

Mouse body weight were measured twice per week until disease onset. Disease onset was considered to be the time at which body weight began to decline from its peak. Treatment was initiated shortly after observation of disease onset.

Initial, IM+IT Treatment:

Before treatment initiation, mice were randomized according to their weight and age into 2 groups of 18 mice each for either treatment with bioreactor-expanded ASC or placebo. Injections were administered at disease onset and 7 days afterwards, in each case.

Intramuscular (IM) Injection.

Mice were injected with 1×10\hat{ }6 cells in 50 mcl (microliters) or PlasmaLyte A (vehicle), in each upper thigh muscle.

Intrathecal (IT) Injection.

Mice were injected with 0.5×10\hat{ }6 cells or PlasmaLyte A in 4-9 mcl in the cerebrospinal (CSF) fluid space in the lumbar L2-L5 segment (the area responsible for hindlimb innervation), under anesthesia.

Subsequent IT Treatment:

In the second half of the experiments, mice were administered ASC by the IT route only, as described in the previous section.

Blood Collection:

Blood was collected and serum separated at 3 time points during the study, then serum was separated, aliquoted into 60 mcl aliquots, and transferred to −80° C. storage:
At the end of the acclimation period, blood was collected from all mice through the orbital sinus.
At day of termination for histological purposes
At time of death (determined as described below)

Time of Death

Mice were considered as dead when unable to roll over within 30 seconds (s) of being placed on their side, and were euthanized at this point.

Results

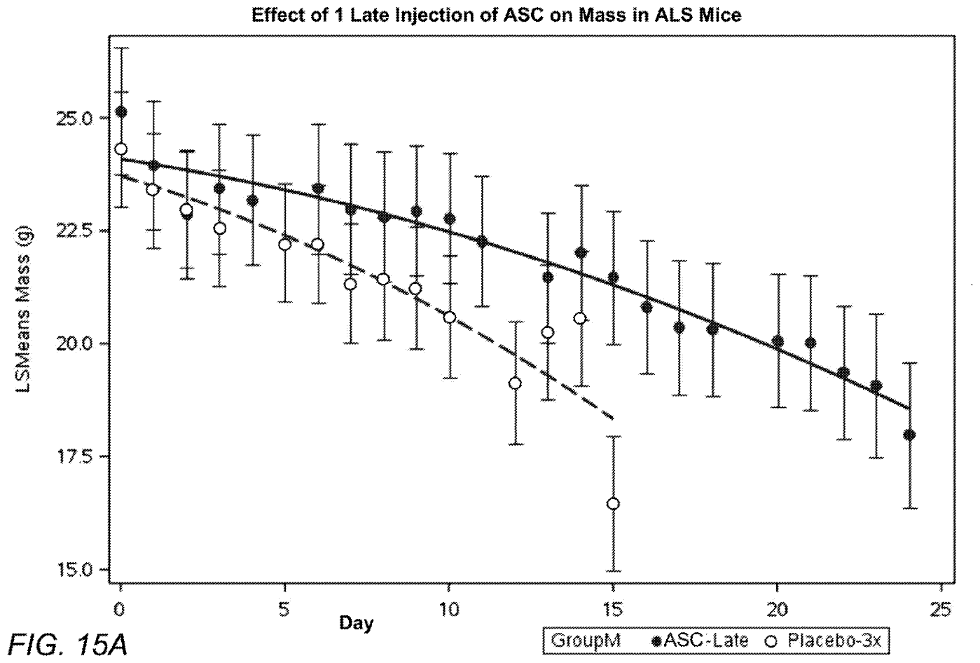

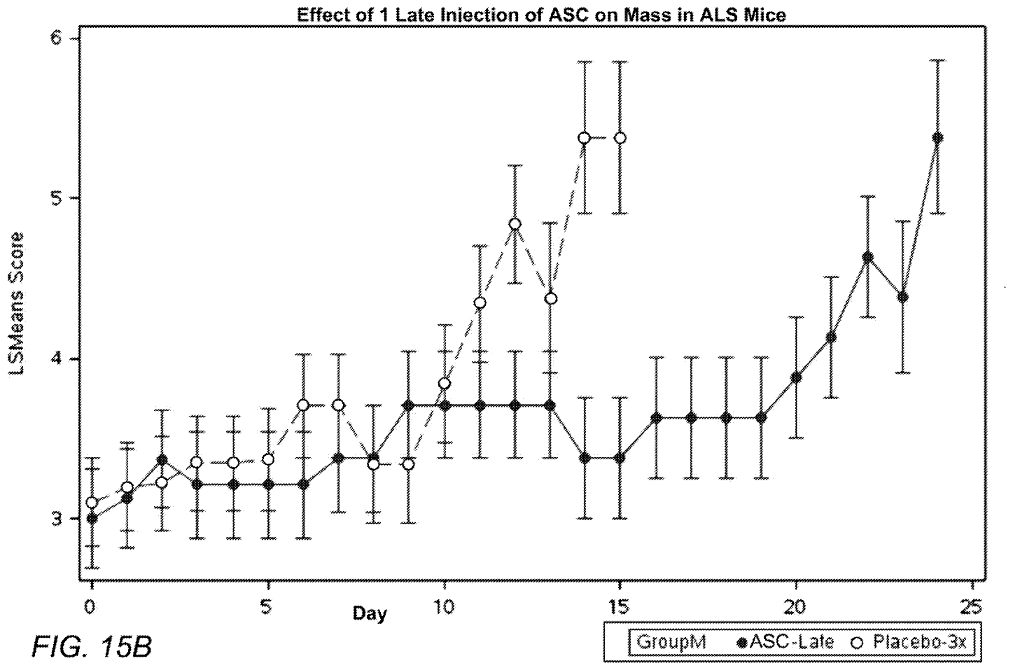

No positive effect was seen from the first round of administration of ASC (by the IT+IM routes). However, a significant positive effect on survival and neurological score was seen after the second round of (IT-only) treatment (FIGS. 15A-B, respectively), with slower decline in body weight and slower progression of disease score (i.e. slower deterioration of motor function)

### Additional Experiments

In still other experiments, ALS model animals or human subjects with ALS are administered induced ASC intranasally, intrathecally, or intravenously, and disease severity and progression is assessed by neurological examination. Improvement of muscle function and elongation of lifespan is evidence of therapeutic efficacy. Other tested parameters may include the effect of treatment on neuro-muscular junction (NMJ) integrity and gastrocnemius (GNS) muscle morphology; and expression of genes and proteins involved in mitochondrial bioenergetics and energy metabolism, antioxidant defense mechanisms, anti-apoptotic indexes and survival pathways in muscle, spinal cord, brain, and blood.

### Example 14: Effect of ASC on Senescence-Accelerated Animals

Methods

SAMP8 mice are obtained from Takeda Industries (Japan).

5×10⁵ viable cells are injected into the hippocampus, half in each hemisphere. The injections are repeated two more times, one and two months after the first injection. Mice are observed daily throughout the study period to determine general well-being and weighed weekly.

Two weeks after the last ASC injection, mice are subjected to the T maze test. Training is continued until mice reach the predetermined end point of five avoidances in six consecutive trials. Cognitive abilities and memory retention are compared between the different groups of ASC-injected mice and control (vehicle-injected, age-matched) mice.

Twenty eight days after the third injection, mice are subjected to an object-place recognition test. Results are expressed as the percent of time spent investigating a novel object in comparison to a familiar object. Cognitive abilities and memory are compared between ASC-injected mice and control mice.

After the last test, mice are anesthetized, and brains are extracted: 8-9 brains in each group are flash frozen and kept for future biochemical analysis, and 4-5 mice in each group are transcardially perfused with 4% paraformaldehyde before brain extraction. After extraction, the perfused brains are kept in formalin for an additional 24 hours and stored in PBS for future immunohistochemical analysis. CSF is also collected at this point. Animals for which injection of ASC improve cognitive function are selected for further investigation towards the illustration of the mechanism of action of the ASC.

Results

SAMP8 mice at an early symptomatic stage of AD (8 months of age) are subject to intra-hippocampal injection of non-induced ASC, induced ASC, or placebo (vehicle). In some experiments, three injections of cells are administered, approximately at monthly intervals. The incidence of human cells in the mouse brains is determined by staining for HuNu, a marker of human nuclei. The effect of induced ASC on disease progression is evaluated with the aversive T maze behavioral test and object place recognition test, to determine the effect of treatment on behavioral and cognitive parameters. Improved performance in cognitive abilities such as learning and memory is indicative of therapeutic efficacy.

### Example 15: Mechanistic Studies of Induced ASC

Methods

Tissue and Fluid Extraction.

Brains are extracted under anesthesia. Some brains in each group are flash frozen and kept for biochemical analysis, while others are transcardially perfused with 4% paraformaldehyde before brain extraction. After extraction, the perfused brains are kept in formalin for an additional 24 hours and stored in phosphate-buffered saline (PBS) for future immunohistochemical analysis. Cerebrospinal fluid (CSF) is also collected at the time of sacrifice.

The hippocampus and the cortex from flash-frozen brains are subject to tissue lysis and homogenization, followed by protein extraction. The following parameters are measured:
Oxidative damage: For detection of protein carbonylation, 2,4-dinitrophenylhydrazine (DNPH) is utilized. For detection of lipid peroxidation-protein bound 4-hydroxynonenal (HNE) is measured. For detection of protein nitrosylation, protein bound 3-nitrotyrosine (3-NT) is measured. Products are detected by slot blot analysis and quantified by densitometry.
AD phenotypic markers: The following markers are measured: APP (amyloid precursor protein), Aβ; hyperphosphorylated tau; GSK-3β (Glycogen Synthase kinase 3β), by western blot analysis and densitometry.

Histological and Immunohistochemical Analysis:

After perfusion with 4% paraformaldehyde, tissue sections containing the hippocampus and the cortex are prepared. Six slices from each brain are analyzed for general histology and for markers of microglial activation by immunofluorescence, including the following parameters:
Microglia activation is measured by immunofluorescent staining for Iba-1 and CD68, which indicated the number of microglia cells and their activation status will serve as a measure of inflammation.
Neuronal loss is measured by quantifying the number of neurons on NeuN-stained slides, assessing dendritic length, and quantifying branching points (with Golgi staining).
Neuro-vascular pathology is measured by detecting microhemorrhages and studying blood vessel area after staining slides with an endothelial cell marker.

In some experiments, brain sections are stained for HuNu (a marker for human nuclei) to detect the persistence and location of the ASC one month after last injection.

CSF Sample Analysis:

Mouse pro- and anti-inflammatory cytokine levels, growth factors, and neurotrophic factors are determined in the CSF, using a mouse custom-made Luminex® panel, including BDNF, GDNF, bFGF, NGF, and VEGF.

Results

Additional biochemical markers are examined in lysates of the hippocampus and the cortex (e.g. protein carbonylation, nitrosylation and lipid peroxidation to measure oxidative stress; lba-1 and CD68 as markers of microglia activation; and classical AD markers (Aβ and phosphorylated tau)). Levels of pro-inflammatory, anti-inflammatory, and neurotrophic factors (of mouse origin) are examined in CSF samples. Optionally, histological analyses of the brains are performed to detect general differences in tissue morphology (i.e. number of neurons/microglia/blood vessels). HuNu (a marker specific to human cell nuclei) is used to stain induced ASC in the brains, to see if the injected cells are still detectably present at specified intervals after the last injection. Improvement in pathological signs, for example decreased oxidative stress, decreased microglial activation, increased number of neurons, or decreased inflammatory markers, is indicative of therapeutic efficacy.

### Example 16: Testing Alternative Routes of Administration of Induced ASC

Induced ASC are administered via various routes, e.g. intracranial, intravenous, intrathecal and intranasal, and therapeutic efficacy of the various routes is compared.

### Example 17: Use of Induced ASC in Treating Alzheimer's Disease

Human subjects with Alzheimer's disease are administered induced ASC intranasally or intravenously and administered memory tests. Improved cognition is evidence of therapeutic efficacy.

### Example 18: Use of Induced ASC in Treating Parkinson's Disease

Induced ASC are tested by intranasal or intrastriatal administration to 6-hydroxydopamine (6-OHDA) rats, which are described, for example, in Naughton et al, and motor deficits are measured in the experimental and control (placebo) groups. In other experiments, induced ASC are tested using the models described in Panicker et al, Holm et al, and the references cited therein. In still other experiments, human subjects with Parkinson's disease are administered induced ASC intranasally or intravenously, and disease severity and progression is assessed by neurological and behavioral examination. Amelioration of the disorder is evidence of therapeutic efficacy.

### Example 19: Use of Induced ASC in Treating Huntington's Disease

Induced ASC are tested by intranasal administration to expanded polyglutamine tract (105Q), which are described, for example, in Yang et al, and neurological examination is performed in the experimental and control (placebo) groups. In other experiments, induced ASC are tested using the models described in Holm et al and the references cited therein. In still other experiments, human subjects with Huntington's disease are administered induced ASC intranasally or intravenously, and disease severity and progression is assessed by neurological examination. Amelioration of the disorder is evidence of therapeutic efficacy.

### Example 20: Use of Induced ASC in Treating Multiple Sclerosis

Induced ASC are tested by intranasal administration to the experimental autoimmune encephalomyelitis (EAE) mice, which are described, for example, in Magliozzi et al, and magnetic resonance imaging (MRI) is performed in the experimental and control (placebo) groups. In other experiments, induced ASC are tested using the models described in Hausler et al and Fan et al, Marino et al, and the references cited in these publications. In still other experiments, human subjects with multiple sclerosis are administered induced ASC intranasally or intravenously, and disease severity and progression is assessed by MRI. Amelioration of the disorder is evidence of therapeutic efficacy.

### Example 21: Induced ASC in Treating Ataxia-Telangiectasia

Induced ASC are tested by intranasal administration in the mouse model described in Duecker R et al and the references cited therein, or the pig ataxia-telangiectasia model described in Holm et al and the references cited therein, and assessment of ataxia is performed in the experimental and control (placebo) groups. In other experiments, human subjects with ataxia-telangiectasia are administered induced ASC intranasally or intravenously, and disease severity and progression is assessed by assessment of ataxia. Amelioration of the disorder is evidence of therapeutic efficacy.

### Example 22: Use of Induced ASC in Treating SMA

Induced ASC are tested by intranasal administration in the mouse model described in Alrafiah A et al and the references cited therein, or the pig SMA model described in Holm et al and the references cited therein, and assessment of muscle strength is performed in the experimental and control (placebo) groups. In other experiments, human subjects with spinal muscular atrophy are administered induced ASC intranasally or intravenously, and disease severity and progression is assessed by assessment of muscle strength. Amelioration of the disorder is evidence of therapeutic efficacy.

### Example 23: Use of Induced ASC in Treating Spinal Cord Injury

Induced ASC are tested by intrathecal or intravenous administration to the mouse spinal cord injury model described in Sugai et al and the references cited therein, and recovery of motor function is followed in the experimental and control (placebo) groups. In other experiments, human subjects with spinal cord injury are administered induced ASC intrathecally, and recovery of motor function is followed. Recovery of motor function is evidence of therapeutic efficacy.

### Example 24: Use of Induced ASC in Treating Spinocerebellar Ataxia (Sac)

ASCs are tested by intranasal, intrathecal, intravascular, intramuscular, intracerebroventricular, intracerebroventricular+intravascular, intrathecal+intravascular, or intranasal+intravascular administration to SCA1-KI mice or rats, which are described, for example, in Mieda et al. Treatment will begin at the age of 4-5 weeks. Treatment is two administrations separated by 2-4 weeks, starting at 5 weeks of age. Motor deficits are measured in the experimental and control (placebo) groups using the MBS functional score, time spent on a Rotarod apparatus and dowel rod walking test. Cerebellar neuropathology to determine Purkinje cell death, atrophy, dendrite complexity will be done at ˜40 weeks. Neuroinflammation in the cerebellum will be determined at 12 weeks. Additionally cortical neuron morphology and pathology will be examined at ˜40 weeks and spinal cord motor neuron pathology will be examined at 12 and/or 20 weeks. Muscle pathology will be tested at 20 and 30 weeks of age. Survival of mice will be followed for up to 1 year. ASC homing into the brain & cerebellum will be examined at 12 and 28 weeks. Growth factor production in the brain & cerebellum will be examined at 12 weeks. Treatment would be considered efficacious if lifespan is lengthened accompanied by reduced cell death and/or reduced neuroinflammation. In other experiments, induced or non-induced ASC are tested using the models described in Zhang et al and the references cited therein. In still other experiments, human subjects with SCA are administered induced ASC intranasally, intrathecally or intravenously, or intrcereboventricularly and disease severity and progression is assessed by neurological examination, improvement of muscle function and elongation of lifespan is evidence of therapeutic efficacy.

### Example 25: Use of Induced ASC in Treating Autism Spectrum Disorders (ASD)

Induced or non-induced ASCs are tested by intranasal, intrathecal, intravascular, intracerbroventricular, intracerbroventricular+intaravascular, intrathecal+intravascular or intranasal+intravascular administration to BTBR mice, which are described, for example, in Perets et al. This model demonstrates autistic-like behavioral phenotypes consistent with the diagnostic criteria for ASD (Ellegood et al). Treatment begins at the age of 6-8 weeks on male mice. Treatment will either be single administration at 6-8 weeks of age or repeated administrations at monthly intervals. Social capabilities will be examined using a battery of behavioral tests as described in Perets et al, Crawley et al, and references cited therein. For example the reciprocal dyadic social recognition test (Segal-Gavish H et al) will be used to determine social interactions during a first encounter with a stranger mouse. Ultrasonic vocalization measurements will be used to determine male-female interactions. The frequency of stereotypical autistic behavior as depicted by increased self-grooming will also be examined. Finally, cognitive rigidity may be measured using a learning test such as the wet T-maze. Behavioral tests will be conducted 1, 3 and 6 months post ASC administration. At each time point mice will be sacrificed, their brains would be excised and levels of BDNF and additional cytokines will be measured in the hippocampus, prefrontal cortex and hypothalamus using either ELISA or Luminex. Neurogenesis will be measured by staining for KI-67 and doublecortin in the hippocampus and the SVZ as described in Segal-Gavish et al. In other experiments, induced or non-induced ASC are tested using the models described in Crawley et al and Belzung et al and the references cited therein. In still other experiments, human subjects with ASD are administered induced or non-induced ASCs intranasally, intrathecally or intravenously and disease severity and progression is assessed by improvement in social communication skills and autism symptoms as described in Dawson et al is evidence of therapeutic efficacy.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References May be Cited in Text

Alrafiah A et al, Plastin 3 Promotes Motor Neuron Axonal Growth and Extends Survival in a Mouse Model of Spinal Muscular Atrophy. Mol Ther Methods Clin Dev. 2018 Jan. 31; 9:81-89. doi: 10.1016/j.omtm.2018.01.007.
Azzouz M, Hottinger A, Paterna J C, Zurn A D, Aebischer P, Büeler H. Increased motoneuron survival and improved neuromuscular function in transgenic ALS mice after intraspinal injection of an adeno-associated virus encoding Bcl-2. Hum Mol Genet. 2000 Mar. 22; 9(5):803-11.
Basso D M, Beattie M S, Bresnahan J C. A sensitive and reliable locomotor rating scale for open field testing in rats. J Neurotrauma. 1995 February; 12(1):1-21.
Belzung C et al, Rodent models for autism: a critical review. Drug Discov Today Dis Models. 2005; 2:93-101.
Betzer, O. et al. Nanoparticle-based CT imaging technique for longitudinal and quantitative stem cell tracking within the brain: application in neuropsychiatric disorders. ACS nano 8, 9274-9285 (2014).
Butterfield, D. A. Beta-Amyloid-associated free radical oxidative stress and neurotoxicity:
implications for Alzheimer's disease. Chemical research in toxicology 10, 495-506 (1997).
Butterfield, D. A. & Poon, H. F. The senescence-accelerated prone mouse (SAMP8): a model of age-related cognitive decline with relevance to alterations of the gene expression and protein abnormalities in Alzheimer's disease. Experimental gerontology 40, 774-783 (2005).
Chase et al, Development and Characterization of a Clinically Compliant Xeno-Free
Culture Medium in Good Manufacturing Practice for Human Multipotent Mesenchymal Stem Cells. Stem Cells Transl Med. 2012 October; 1(10): 750-758.
Cho, J. H. & Johnson, G. V. Primed phosphorylation of tau at Thr231 by glycogen synthase kinase 3beta (GSK3beta) plays a critical role in regulating tau's ability to bind and stabilize microtubules. Journal of neurochemistry 88, 349-358 (2004).
Clayton A et al, Analysis of antigen presenting cell derived exosomes, based on immuno-magnetic isolation and flow cytometry. J Immunol Methods. 2001; 247(1-2):163-74.
Clinton, L. K., Blurton-Jones, M., Myczek, K., Trojanowski, J. Q. & LaFerla, F. M. Synergistic Interactions between Abeta, tau, and alpha-synuclein: acceleration of neuropathology and cognitive decline. The Journal of Neuroscience: the official journal of the Society for Neuroscience 30, 7281-7289 (2010).
Crescitelli R et al, Distinct RNA profiles in subpopulations of extracellular vesicles: apoptotic bodies, microvesicles and exosomes. J Extracell Vesicles. 2013 Sep. 12; 2.
Dawson G et al, Autologous Cord Blood Infusions Are Safe and Feasible in Young Children with Autism Spectrum Disorder: Results of a Single-Center Phase I Open-Label Trial. Stem Cells Transl Med. 2017 Apr. 5. doi: 10.1002/sctm.16-0474.
de Pedro N et al, Protective effects of isolecanoric acid on neurodegenerative in vitro models. Neuropharmacology. 2016 February; 101:538-48.
Dominici et al. Minimal criteria for defining multipotent mesenchymal stromal cells. The International Society for Cellular Therapy position statement. Cytotherapy. 2006; 8(4):315-7.
Duecker R et al, Oxidative stress-driven pulmonary inflammation and fibrosis in a mouse model of human ataxia-telangiectasia. Redox Biol. 2018 April; 14:645-655. doi: 10.1016/j.redox.2017.11.006.
Ellegood J, Crawley J N. Behavioral and Neuroanatomical Phenotypes in Mouse Models of Autism. Neurotherapeutics. 2015 July; 12(3):521-33. doi: 10.1007/s13311-015-0360-z.
Fan et al, Follicular Helper CD4+ T Cells in Human Neuroautoimmune Diseases and Their Animal Models. Mediators Inflamm. 2015; 2015:638968. doi: 10.1155/2015/638968. Epub 2015 Aug. 2.
Farr, S. A. et al. The antioxidants alpha-lipoic acid and N-acetylcysteine reverse memory impairment and brain oxidative stress in aged SAMP8 mice. Journal of neurochemistry 84, 1173-1183 (2003).
Farr, S. A. et al. Antisense oligonucleotide against GSK-3beta in brain of SAMP8 mice improves learning and memory and decreases oxidative stress: Involvement of transcription factor Nrf2 and implications for Alzheimer disease. Free radical biology & medicine 67, 387-395 (2014).
Flood, J. F. & Morley, J. E. Age-related changes in footshock avoidance acquisition and retention in senescence accelerated mouse (SAM). Neurobiology of aging 14, 153-157 (1993).
Flood, J. F., Harris, F. J. & Morley, J. E. Age-related changes in hippocampal drug facilitation of memory processing in SAMP8 mice. Neurobiology of aging 17, 15-24 (1996).
Flood, J. F., Farr, S. A., Uezu, K. & Morley, J. E. Age-related changes in septal serotonergic, GABAergic and glutamatergic facilitation of retention in SAMP8 mice. Mechanisms of ageing and development 105, 173-188 (1998).
Gurney M E (1997) The use of transgenic mouse models of amyotrophic lateral sclerosis in preclinical drug studies. J Neurol Sci 152 Suppl 1: S67-73.
Häusler et al, Natalizumab analogon therapy is effective in a B cell-dependent multiple sclerosis model. Neuropathol Appl Neurobiol. 2015 October; 41(6):814-31. doi: 10.1111/nan.12220. Epub 2015 May 2.
Heneka, M. T. & O'Banion, M. K. Inflammatory processes in Alzheimer's disease. Journal of neuroimmunology 184, 69-91 (2007).
Holm et al, Genetically Modified Pig Models for Neurodegenerative Disorders. J Pathol. 2015 Oct. 8. doi: 10.1002/path.4654.
Kinzebach S and Bieback K. Expansion of Mesenchymal Stem/Stromal cells under xenogenic-free culture conditions. Adv Biochem Eng Biotechnol. 2013; 129:33-57.
Kunis et al, Immunization with a Myelin-Derived Antigen Activates the Brain's Choroid Plexus for Recruitment of Immunoregulatory Cells to the CNS and Attenuates Disease Progression in a Mouse Model of ALS. J Neurosci. 2015 Apr. 22; 35(16):6381-93.
Magliozzi R., Columba-Cabezas S., Serafini B., Aloisi F. Intracerebral expression of CXCL13 and BAFF is accompanied by formation of lymphoid follicle-like structures in the meninges of mice with relapsing experimental autoimmune encephalomyelitis. Journal of Neuroimmunology. 2004; 148(1-2): 11-23.
Marino et al, Differences in protein quality control correlate with phenotype variability in 2 mouse models of familial amyotrophic lateral sclerosis. Neurobiol Aging. 2015 January; 36(1):492-504.
Mathias R A et al, Isolation of extracellular membranous vesicles for proteomic analysis. Methods Mol Biol. 2009; 528:227-42.
Mathis S et al, Current view and perspectives in amyotrophic lateral sclerosis. Neural Regen Res. 2017 February; 12(2):181-184. doi: 10.4103/1673-5374.200794.
Mathieu et al, New behavioral protocols to extend our knowledge of rodent object recognition memory. Learn Mem. 2010 August; 17(8): 407-419.
Mechan et al, A comparison between Dark Agouti and Sprague-Dawley rats in their behaviour on the elevated plus-maze, open-field apparatus and activity meters, and their response to diazepam. Psychopharmacology (Berl). 2002 January; 159(2):188-95.
Mieda T et al, Mesenchymal stem cells attenuate peripheral neuronal degeneration in spinocerebellar ataxia type 1 knockin mice. J Neurosci Res. 2016 March; 94(3):246-52. doi: 10.1002/jnr.23698. Epub 2015 Dec. 28
Mendez et al, c-Fos expression correlates with performance on novel object and novel place recognition tests. Brain Res Bull. 2015 August; 117:16-23.
Mimura et al, Growth factor-defined culture medium for human mesenchymal stem cells. Int. J. Dev. Biol. 55: 181-187 (2011).
Mondragon-Rodriguez, S., Perry, G., Zhu, X., Moreira, P. I. & Williams, S. Glycogen synthase kinase 3: a point of integration in Alzheimer's disease and a therapeutic target? International journal of Alzheimer's disease 2012, 276803 (2012).
Morley, J. E., Armbrecht, H. J., Farr, S. A. & Kumar, V. B. The senescence accelerated mouse (SAMP8) as a model for oxidative stress and Alzheimer's disease. Biochimica et biophysica acta 1822, 650-656 (2012).
Mosher, K. I. et al. Neural progenitor cells regulate microglia functions and activity. Nature neuroscience 15, 1485-1487 (2012).
Naughton et al, Differential pattern of motor impairments in neurotoxic, environmental and inflammation-driven rat models of Parkinson's disease. Behav Brain Res. 2015 September 19. pii: S0166-4328(15)30198-4. doi: 10.1016/j.bbr.2015.09.025.
Ouhaz et al, Behavioral and cognitive changes after early postnatal lesions of the rat mediodorsal thalamus. Behav Brain Res. 2015 October 1; 292:219-32.
Panicker et al, Fyn Kinase Regulates Microglial Neuroinflammatory Responses in Cell Culture and Animal Models of Parkinson's Disease. J Neurosci. 2015 Jul. 8; 35(27):10058-77.
Perets N et al, Long Term Beneficial Effect of Neurotrophic Factors-Secreting Mesenchymal Stem Cells Transplantation in the BTBR Mouse Model of Autism.
Behav Brain Res. 2017 April 6. pii: S0166-4328(17)30241-3. doi: 10.1016/j.bbr.2017.03.047.
Rajaraman G et al, Optimization and scale-up culture of human endometrial multipotent mesenchymal stromal cells: potential for clinical application. Tissue Eng Part C Methods. 2013 January; 19(1): 80-92.
Segal-Gavish H et al, Mesenchymal Stem Cell Transplantation Promotes Neurogenesis and Ameliorates Autism Related Behaviors in BTBR Mice. Autism Res. 2016 January; 9(1):17-32. doi: 10.1002/aur.1530. Epub 2015 Aug. 10.
Sugai et al, Neural stem/progenitor cell-laden microfibers promote transplant survival in a mouse transected spinal cord injury model. J Neurosci Res. 2015 December; 93(12):1826-38.
Sultana, R., Perluigi, M. & Allan Butterfield, D. Lipid peroxidation triggers neurodegeneration: a redox proteomics view into the Alzheimer disease brain. Free Radical Biology & Medicine 62, 157-169 (2013).
Valko, M. et al. Free radicals and antioxidants in normal physiological functions and human disease. The International Journal of Biochemistry & Cell Biology 39, 44-84 (2007).
Yang et al, Expression of Huntington's disease protein results in apoptotic neurons in the brains of cloned transgenic pigs. Hum Mol Genet. 2010 Oct. 15; 19(20):3983-94.
Zhang M J et al, Human umbilical mesenchymal stem cells enhance the expression of neurotrophic factors and protect ataxic mice. Brain Res. 2011 Jul. 21; 1402:122-31. doi: 10.1016/j.brainres.2011.05.055. Epub 2011 May 30.

What is claimed is:

1. A method of treating a neurodegenerative disease in a subject in need thereof, comprising the step of administering to said subject a pharmaceutical composition comprising induced adherent stromal cells (ASC), thereby treating a neurodegenerative disease.

2. The method of claim 1, wherein said neurodegenerative disease is Alzheimer's disease.

3. The method of claim 1, wherein said neurodegenerative disease is Parkinson's disease.

4. The method of claim 1, wherein said neurodegenerative disease is Amyotrophic lateral sclerosis (ALS).

5. The method of claim 1, wherein said neurodegenerative disease is Huntington's disease.

6. The method of claim 1, wherein said neurodegenerative disease is multiple sclerosis (MS), spinal muscular atrophy, spinal cord injury, spinocerebellar ataxia, or an autism spectrum disorder.

7-10. (canceled)

11. The method of claim 1, wherein said administering is selected from intranasal administration, intracerebral administration, intracerebroventricular administration, intrathecal administration, intravenous administration, and intramuscular administration.

12. The method of claim 1, wherein said ASC have been induced by incubation in an induction medium comprising heparin and cyclic AMP (cAMP) or an analogue thereof.

13. The method of claim 12, wherein said induction medium further comprises an induction agent selected from basic fibroblast growth factor (b-FGF), PDGF (platelet-derived growth factor), and Neuregulin.

14-17. (canceled)

18. The method of claim 12, wherein said induction medium further comprises serum.

19. The method of claim 12, wherein said induction medium is serum free.

20. The method of claim 12, wherein said ASC were expanded ex vivo prior to inducing said ASC.

21. The method of claim 20, wherein said ASC are expanded on a 2D substrate, and then induced on a 3D substrate.

22. The method of claim 12, wherein said ASC have been incubated in a serum-free medium, prior to incubation in said induction medium.

23. The method of claim 1, wherein said ASC have been induced by incubation in a serum-free medium comprising PDGF, bFGF, and TGF β.

24. (canceled)

25. The method of claim 23, wherein said medium further comprises cAMP.

26. The method of claim 23, wherein said ASC have been incubated on a 3D substrate, following said incubation in a serum-free medium, wherein said 3D substrate culture apparatus comprises a synthetic adherent material.

27-31. (canceled)

32. A method of inducing ASC to secrete a neurotrophic or neuroprotective growth factor, comprising incubating said ASC in an induction medium comprising heparin and cAMP or a cAMP analogue.

33. The method of claim 32, wherein said induction medium further comprises an induction agent selected from basic fibroblast growth factor (b-FGF), PDGF (platelet-derived growth factor), and Neuregulin.

34-45. (canceled)

46. The method of claim 1, wherein said ASC originate from placental tissue.

47-50. (canceled)
